(19) United States
(12) Patent Application Publication
(10) Pub. No.: US 2026/0325658 A1
(43) Pub. Date: Oct. 22, 2026

(54) DENDRIMER COMPOSITIONS FOR TARGETED DELIVERY OF PSYCHEDELIC THERAPEUTICS

(71) Applicant: The Johns Hopkins University, Baltimore, MD (US)

(72) Inventors: Kannan Rangaramanujam, Highland, MD (US); Sujatha Kannan, Highland, MD (US); Kunal Parikh, Reynoldsburg, OH (US); Anjali Sharma, Baltimore, MD (US); Wathsala Liyanage, Ellicott City, MD (US); Preeti Vyas, Baltimore, MD (US)

(21) Appl. No.: 19/106,539

(22) PCT Filed: Aug. 25, 2023
(86) PCT No.: PCT/US2023/072922

§ 371 Date: Feb. 25, 2025

Related U.S. Application Data
(60) Provisional application No. 63/401,470, filed on Aug. 26, 2022.

(51) Int. Cl. A61K 47/60; A61K 47/54; A61P 25/28

(52) U.S. Cl. CPC A61K 47/60; A61K 47/549; A61P 25/28

(57) ABSTRACT

Dendrimer formulations including one or more psychedelic or hallucinogenic agents, and methods of use thereof are described. Preferably, the dendrimer-conjugated agents selectively bind to one or more receptors on the surface or inside the target cells. The formulations are suitable for enteral and/or parenteral delivery for treating one or more receptor mediated disorders, including psychological, cognitive, behavioral, and/or mood disorders.

### CROSS-REFERENCE TO RELATED APPLICATIONS

This application claims benefit of U.S. Provisional Application 63/401,470 filed Aug. 26, 2022 entitled “DENDRIMER COMPOSITIONS FOR TARGETED DELIVERY OF PSYCHEDELIC THERAPEUTICS” by The Johns Hopkins University listing inventors Kannan Rangaramanujam, Kunal Parikh, Sujatha Kannan, and Anjali Sharma, hereby incorporated in entirety.

### FIELD OF THE INVENTION

This invention is generally in the field of hallucinogenic drug formulations, more specifically dendrimer hallucinogen drug-conjugates, that improve receptor binding, cell selectivity and reduce side effects.

### STATEMENT REGARDING FEDERALLY SPONSORED RESEARCH

None.

### BACKGROUND OF THE INVENTION

The word “psychedelic” (psyche (i.e., the mind or soul) and delos (i.e., to show)) was first coined by psychiatrist Humphry Osmond in 1956, who had been conducting research on lysergic acid diethylamide (LSD) at the time. Psychedelic drugs such as N,N-DMT/DMT (N,N-dimethyltryptamine), 5-MeO-DMT (5-methoxy-N,N-dimethyltryptamine), LSD (lysergic acid diethylamide), MDMA (3,4-methylenedioxymethamphetamine) and psilocybin have had significant value as an entheogen in spiritual, religious (shamanic) and sociocultural rituals in Central and South American cultures for thousands of years.

Up until recently, the spiritual, religious and medicinal value of these drugs could not be explored in a scientific context. More recently, a second wave of psychedelic research is now focusing on psychedelics as neuropharmaceuticals to treat alcohol and tobacco addiction, general mood and anxiety disorders and cancer-related depression. Psychedelics and other classes of compounds associated with them (e.g., entactogens which are known for temporarily altering consciousness, involving acute changes in somatic, perceptual, cognitive properties) are receiving increased attention. This is partly attributable to their wider usage, new synthesis and purification approaches, improved mechanistic understanding, and ongoing clinical trials. Their profound potential ability to impact thought and consciousness also makes it important to understand their mechanism of action, and to target their action to cells and tissue regions of interest, avoiding side effects.

Psychedelic and hallucinogenic drugs, such as plant-derived indoleamines (e.g., N,N-dimethyltryptamine (DMT), 5-methoxy-DMT (5-MeO-DMT), psilocybin, 4-hydroxy-DMT (psilocin, the active metabolite of psilocybin)), phenylalkylamines (e.g., mescaline and synthetic ‘amphetamines’ such as 2,5-dimethoxy-4-iodoamphetamine (DOI) and 2,5-dimethoxy-4-bromoamphetamine (DOB)), mianserin and semi-synthetic ergolines (e.g., LSD), have shown significant therapeutic potential for treatment of several mental health and neurological disorders (e.g., depression, treatment resistant depression, suicidal ideation, autism, bi-polar disorder, anxiety, drug dependence, substance abuse disorders, post-traumatic stress disorder, obesity, headaches, pain, fibromyalgia, obsessive compulsive disorder, anorexia nervosa, inflammation, Alzheimer's, attention-deficit/hyperactivity disorder, narcolepsy).

In general, psychedelic molecules achieve therapeutic effects via activation of 5-HT2A receptors in the cortex of the brain. 5-HT2A is a type of serotonin receptor which is thought to mediate brain plasticity, and is found with high density in areas of the cortex involved in high-level cortical processing (prefrontal cortical regions: cingulate cortex and posterior cingulate cortex). However, there are significant challenges for practical implementation of these potential therapeutics, including solubility, bioavailability, absorption, side effects/toxicity, time to efficacy, duration of efficacy, and hallucinogenic effects. Psychedelics have limited access into these critical regions of the brain, and specifically to brain cells and immune cells which are involved in disease processes. Collectively, these issues limit the applicability, efficacy, and translational potential of this class of drugs, and prevent them from realizing their broad therapeutic potential.

It is therefore an object of the present invention to provide formulations allowing for more selective delivery of psychedelic and hallucinogenic drugs.

### SUMMARY OF THE INVENTION

The group of hallucinogens and dendrimer compositions formulated herein include active ingredients such as: (i) psychedelics, group of serotonergic, typically Schedule I, agonists such as psilocybin, lysergic acid, mescaline; (ii) entactogens, Schedule I monoamine releasers and reuptake inhibitors known to evoke a sense of emotional openness and connection such as 3,4-methylenedioxy-methamphetamine (MDMA), and 3,4-methylenedioxyamphetamine (MDA); (iii) dissociatives, glutamatergic NMDA antagonists such as ketamine, dextromethorphan (DXM) and nitrous oxide; (iv) atypical hallucinogens, with diverse mechanisms such as Δ⁹-tetrahydrocannabinol (THC) and ibogaine. Dendrimer conjugate compositions that can deliver drugs to receptors on specific cells (neuronal cells, glial cells, macrophages), including targets on their surface and inside them, have been developed. These formulations can enhance the effectiveness of these drugs through superior binding to target receptors, enabling lower doses, leading to new mechanistic insights, reducing side effects, improving solubility, formulation, PK and other aspects of the use of these drugs, opening new clinical avenues of use.

Formulations are based on conjugates of the drugs to dendrimers, especially PAMAM (such as G3, G4, G5, and G6 hydroxyl-terminated PAMAM dendrimers) and glucose dendrimers (such as G1, G2, and G3 glucose dendrimers). The dendrimers provide enhanced solubility, uptake into the brain and other specific cell types, and selectivity of uptake and receptor binding.

### BRIEF DESCRIPTION OF THE DRAWINGS

FIGS. 1A and 1B are schematics showing an exemplary synthetic route for dendrimer-psilocin with cleavable ester linkage using click chemistry. Psilocin is first conjugated to a linker with an azide moiety via an ester linkage (FIG. 1A), followed by conjugation to a dendrimer modified with surface alkyne groups via azide-alkyne click reactions (FIG. 1B).

FIGS. 2A and 2B are schematics showing an exemplary synthetic route for dendrimer-psilocin analog with non-cleavable amide linkage using click chemistry. Psilocin analog is first conjugated to a linker with an azide moiety via an amide linkage (FIG. 2A), followed by conjugation to a dendrimer modified with surface alkyne groups via azide-alkyne click reactions (FIG. 2B).

FIGS. 3A and 3B are schematics showing an exemplary synthetic route for dendrimer-ketamine with non-cleavable amino-alkyl linkage using copper-catalyzed alkyne azide click chemistry. Ketamine hydrochloride (1) is first modified with an alkyne (FIG. 3A), followed by conjugation to a dendrimer modified with surface azide groups via azide-alkyne click reactions (FIG. 3B).

FIGS. 4A and 4B are schematics showing an exemplary synthetic route for dendrimer-DMT analog with non-cleavable amide linkage using copper-catalyzed alkyne azide click chemistry. N,N dimethyl tryptamine analog (DMT analog) is first conjugated to a linker with an azide moiety via an amide linkage (FIG. 4A), followed by conjugation to a dendrimer modified with surface alkyne groups via azide-alkyne click reactions (FIG. 4B).

FIGS. 5A and 5B are schematics of the synthesis of dendrimer-DMT with a non-cleavable amino-alkyl linkage. FIG. 5A shows DMT drug modified with an alkyne group. FIG. 5B shows conjugation to a dendrimer modified with surface azide groups via azide-alkyne click reactions.

FIGS. 6A and 6B are schematics of the dendrimer-lysergic acid diethylamide (dendrimer-LSD) with a non-cleavable amino-alkyl linkage. FIG. 6A shows LSD modified with an alkyne group. FIG. 6B shows conjugation to a dendrimer modified with surface azide groups via azide-alkyne click reactions.

FIG. 7 is a schematic of the stepwise synthetic route for the synthesis of glucose dendrimer-psilocin conjugate with a cleavable ester linkage.

FIG. 8 is a schematic of the stepwise synthetic route for the synthesis of glucose dendrimer-psilocin analog conjugate with a non-cleavable amide linkage.

FIG. 9 is a schematic of the stepwise synthetic route for the synthesis of glucose dendrimer-ketamine conjugate with a non-cleavable amino-alkyl linkage.

FIG. 10 is a schematic of the stepwise synthetic route for the synthesis of glucose dendrimer N,N dimethyl tryptamine analog (DMT analog) conjugate with a non-cleavable amide linkage.

FIG. 11 is a schematic of the stepwise synthetic route for the synthesis of glucose dendrimer-DMT analog conjugate with a non-cleavable amino-alkyl linkage.

FIG. 12 is a schematic of the stepwise synthetic route for the synthesis of glucose dendrimer-lysergic acid diethylamide (LSD) conjugate with a non-cleavable amino-alkyl linkage.

FIG. 13 is a schematic overview of the main pharmacological targets of LSD, psilocybin, DMT, MDMA, and ketamine, the signaling cascades involved, hormonal modulation, as well as main behavioral outcomes following their administration in both animals and humans.

FIG. 14A is a schematic of the synthesis of a PAMAM dendrimer-norketamine conjugate. FIG. 14B is a schematic of the synthesis of a Glucose dendrimer-norketamine conjugate.

FIG. 15A is a graph of an NMDAR 1A/2B antagonist assay for glucose dendrimer-ketamine (IC50=4.54 μM), hydroxyl dendrimer-ketamine (IC50>100), and norketamine (IC50=6.96 μM). FIG. 15B is the % binding efficacy of the log concentration of compound in micromolar in a D2L human dopamine GPCR cell based agonist cAMP assay. Norketamine (solid circle), glucose dendrimer-ketamine EC50=13.08 micromolar (open circle), and hydroxyl dendrimer-ketamine EC50=4.263 micromolar (triangle). FIG. 15C is the % efficacy of the log concentration of ketamine in micromolar in the TAI human trace amine GPCR cell based agonist CAMP assay. Norketamine (solid circle), glucose dendrimer-ketamine EC50=13.08 micromolar (open circle), and hydroxyl dendrimer-ketamine EC50=4.263 micromolar (triangle).

FIGS. 16A and 16B are graphs of wild type, knock out saline (controls) versus knockout mice treated with dendrimer-ketamine conjugate composite neurobehavior score of (FIG. 16A) and probability of survival over post natal day (FIG. 16B). FIG. 16C is a graph of the distance traveled (m); FIG. 16D is a graph of the speed at which the mice traveled; FIG. 16E is a graph of the time spent in corners.

### DETAILED DESCRIPTION OF THE INVENTION

### I. Definitions

The term “hallucinogen” refer to a group of chemically heterogeneous compounds, all with the ability to induce altered states of consciousness (ASC) characterized by profound alterations in mood, thought processes, perception, and experience of the self and environment otherwise rarely experienced except in dreams, contemplative and religious exaltation, and acute psychoses. Not all hallucinogenic compounds reliably produce visual and auditory hallucinations. Therefore, hallucinogens may also be referred to as psychotomimetic (psychosis-mimicking), psycholytic (psyche-loosening), or psychedelic (mind-manifesting), reflecting the widely different attitudes and intentions with which these substances have been approached.

The term “psychedelics” refer to a subclass of hallucinogenic drugs whose primary effect is to trigger non-ordinary states of consciousness. This causes specific psychological, visual, and auditory changes, and often a substantially altered state of consciousness. Psychedelic states are often compared to meditative, psychodynamic or transcendental types of alterations of mind. The “classical” psychedelics, the psychedelics with the largest scientific and cultural influence, are mescaline, LSD, psilocybin, and DMT.

Most psychedelic drugs fall into one of the three families of chemical compounds: tryptamines, phenethylamines, or lysergamides and many tend to act via serotonin 2A receptor agonism. When compounds bind to serotonin 5-HT2A receptors, they modulate the activity of key circuits in the brain involved with sensory perception and cognition, however, the exact nature of how psychedelics induce changes in perception and cognition via the 5-HT2A receptor is still unknown, although reduction in default mode network activity and increased functional connectivity between regions in the brain as a result may be one of the most relevant pharmacological mechanisms underpinning the psychedelic experience, particularly ego death.

The terms “active agent” or “biologically active agent” are used interchangeably to refer to a chemical or biological compound that induces a desired pharmacological and/or physiological effect, which may be prophylactic, therapeutic, or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs. The term “therapeutic agent” refers to an agent that can be administered to treat one or more symptoms of a disease or disorder. The term “diagnostic agent” generally refers to an agent that can be administered to reveal, pinpoint, and define the localization of a pathological process. The diagnostic agents can label target cells that allow subsequent detection or imaging of these labeled target cells

“Analog” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered an analog of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified compound, if it induces the same pharmacological effect, physiological effect, or both, as the specified compound. “Analog” can also refer to a modification including, but not limited to, hydrolysis, reduction, or oxidation products, of the compounds. Hydrolysis, reduction, and oxidation reactions are known in the art.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases.

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more inhibitors may inhibit or reduce the activity and/or quantity of diseased neurons by about 10%, 20%, 30%, 40%, 50%, 75%, 85%, 90%, 95%, or 99% from the activity and/or quantity of the same cells in equivalent tissues of subjects that did not receive, or were not treated with the dendrimer compositions. In some embodiments, the inhibition and reduction are compared at levels of mRNAs, proteins, cells, tissues, and organs. For example, an inhibition and reduction in the rate of neural loss, in the rate of decrease of brain weight, or in the rate of decrease of hippocampal volume, as compared to an untreated control subject.

The term “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with depression are mitigated or eliminated, including, but are not limited to, reducing the level of anxiety, agitation, or restlessness, improving feelings of sadness, tearfulness, emptiness or hopelessness, increasing the quality of life of those suffering from the disease, decreasing the dose of other medications required to treat the disease, delaying the progression of the disease.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent, or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “biodegradable” generally refers to a material that will degrade or erode under physiologic conditions to smaller units or chemical species that are capable of being metabolized, eliminated, or excreted in vivo. The degradation time is a function of composition and morphology.

The term “dendrimer” includes, but is not limited to, a molecular architecture with an interior core, interior layers, or “generations” of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific location. The moiety may be, for example, a protein, nucleic acid, nucleic acid analog, carbohydrate, or small molecule. The location may be a tissue, a particular cell type, a subcellular compartment, or a molecule such as a receptor.

The term “prolonged residence time” refers to an increase in the time required for an agent to be cleared from a patient's body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types.

The terms “incorporated” and “encapsulated” refer to incorporating, formulating, or otherwise including an agent into and/or onto a composition that allows for release, such as sustained release, of such agent in the desired application. The agent or other material can be incorporated into a dendrimer, by binding to one or more surface functional groups of such dendrimer (by covalent, ionic, or other binding interaction), by physical admixture, by enveloping the agent within the dendritic structure, and/or by encapsulating the agent inside the dendritic structure.

As used herein, central nervous system (“CNS”) includes the brain and spinal cord. As used herein, peripheral nervous system (“PNS”) refers to the nerves other than in the brain and spinal cord.

“Hydroxyl-terminated,” as relates to dendrimers, refers to dendrimers that have a hydroxyl group on their surface. These hydroxyl groups are not attached to the termini of the dendrimers via a sugar moiety (such as a saccharide moiety).

“Sugar-terminated,” as relates to dendrimers, refers to dendrimers that contain a sugar moiety (such as a saccharide moiety) on their surface and not in their core.

“Sugar-based,” as relates to dendrimers, refers to dendrimers that contain a sugar moiety (such as a saccharide moiety) in their core, or their core and on their surface.

### II. Compositions

Compositions of dendrimers conjugated or complexed with one or more hallucinogens and/or dissociative compounds for preventing and/or treating symptoms associated with one or more psychological, cognitive, behavioral, mood disorders, and/or non-neurological disorder, in a subject in need thereof, have been developed. The compositions are particularly suited for treating and/or ameliorating one or more symptoms of mental health and neurological disorders (e.g., depression (major depressive disorder, treatment-resistant depression, post-partum depression), suicidal ideation, autism, bi-polar disorder, anxiety, drug dependence, substance abuse disorders, post-traumatic stress disorder, obesity, headaches, cluster headaches, migraines, epilepsy, pain, fibromyalgia, obsessive compulsive disorder, anorexia nervosa, inflammation, Alzheimer's, attention-deficit/hyperactivity disorder, narcolepsy, Tourette's syndrome). In preferred embodiments, dendrimers are glucose dendrimers or hydroxyl terminated dendrimers such as hydroxyl terminated PAMAM or sugar modified dendrimers.

Exemplary psychedelics include psilocin, ketamine (R-ketamine, S-ketamine, (R/S)-ketamine), norketamine, ketamine analogues, ketamine metabolites, N,N dimethyl tryptamine (DMT), 4-acetoxy-N,N-dimethyl tryptamine, 5-methoxy DMT, 5-chloro DMT, lysergide (LSD), 3,4-methylenedioxymethamphetamine (MDMA), psilocybin, ibogaine, mescaline, mianserin and norbaeocystin.

Generally, the hallucinogens and/or their derivatives bind to a receptor on the surface of the target cells and/or a receptor inside the target cells. Exemplary target cells include, but not limited to, brain cells such as microglia, astrocytes, and/or neurons, for example, those within the site of pathology in the brain or the CNS; cells in the peripheral nervous system, such as peripheral neurons, glia, and/or their supporting cells, e.g., enteric cells, cardiovascular cells, and immune system cells. The microglia and/or astrocytes to which the hallucinogens and/or their derivatives are delivered may be activated or inactive microglia and/or astrocytes. Classic/serotonergic psychedelic compounds also display immunomodulatory properties, and therefore have applications in autoimmune disorders.

The hallucinogens and/or their derivatives of the dendrimer-active agent conjugate binds to a target receptor on the surface of the target cell or inside the target cell. In some embodiments, when hallucinogens and/or their derivatives binds to the target receptor, the agent remains conjugated to the dendrimer. In these embodiments, following binding, the agent may be released from the dendrimer or remain conjugated to the dendrimer as an intact dendrimer-active agent conjugate. In some embodiments, the hallucinogens and/or their derivatives are released from the dendrimer at close proximity to the target receptor and then binds to the target receptor on the target neural and/or glial cell.

### A. Dendrimers

Dendrimers are three-dimensional, hyperbranched, monodispersed, globular and polyvalent macromolecules including surface end groups (Tomalia, D. A., et al., Biochemical Society Transactions, 35, 61 (2007); and Sharma, A., et al., ACS Macro Letters, 3, 1079 (2014)).

The term “dendrimer” includes, but is not limited to, a molecular architecture with an interior core (“G0”) and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

Generally, the dendrimers have a diameter between about 1 nm and about 60 nm, more preferably between about 1 nm and about 50 nm, between about 1 nm and about 40 nm, between about 1 nm and about 30 nm, between about 1 nm and about 20 nm, between about 1 nm and about 10 nm, or between about 1 nm and about 5 nm. In some embodiments, the diameter is between about 1 nm to about 2 nm. The preferred size of the dendrimers for crossing the blood brain barrier (“BBB”) is less than 5 nm, whereas those for not crossing the BBB and staying in the peripheral circulation are greater than 5 nm. In some embodiments, the dendrimers have a diameter effective to penetrate BBB and to be retained close to or within target neural and/or glial cells for delivery of the agents conjugated thereto. In some embodiments, the dendrimers have a diameter effective to penetrate a BBB and to be internalized into target neural and/or glial cells for delivery of the agents conjugated thereto, such as for example, neurons, oligodendrocytes, astrocytes, microglial, and neuroglial support cells. In some embodiments, the dendrimers have a diameter effective to penetrate a barrier interface, such as a blood nerve barrier (“BNB”), and to be internalized into neural and/or glial cells of the peripheral nervous system for delivery of the agents conjugated thereto such as for example, neurons, Schwann cells, satellite cells, and neuroglial support cells. In some embodiments, the dendrimers have a diameter effective to be retained in the peripheral circulation for delivery of the agents conjugated thereto to target cells of the peripheral nervous system e.g., enteric neurons and glia. A major benefit of the use of dendrimer conjugates is the ability of the dendrimer to enhance the binding of the psychedelic drug to its target receptor on target cells, for example, binding of compounds to the serotogenic receptors on neurons in the affected areas of the brain.

In some embodiments, dendrimers have a molecular weight between about 500 Daltons and about 100,000 Daltons inclusive, between about 500 Daltons and about 50,000 Daltons inclusive, or between about 1,000 Daltons and about 20,000 Daltons inclusive. Dendrimer sizes of less than 30,000 Da are preferred for transport across the BBB, and sizes of greater than 50,000 Da are preferred for confinement to the periphery.

In some embodiments, the dendrimers have a hypercore (e.g., dipentaerythritol) and one or more monosaccharide branching units. In some embodiments, the monosaccharide branching units are conjugated to the core or the prior layer of monomers via linkers such as polyethylene glycol chains. In preferred embodiments, the hypercore is dipentaerythritol and the monosaccharide branching unit is glucose-based branching unit such as shown in Structures II-IV.

In the most preferred embodiment, the dendrimers are made entirely of glucose building blocks. PAMAM dendrimers modified by sugar may also work, but dendrimers made of sugars, especially glucose, are most preferred. Particularly preferred glucose dendrimers are G1 to G3 glucose dendrimers, such as G1, G2, and/or G3 glucose dendrimers.

Suitable dendrimers scaffolds for use in the conjugates include, but are not limited to, poly(amidoamine), also known as PAMAM, or STARBURST™ dendrimers; polypropylamine (POPAM), polyethylenimine, polylysine, polyester, iptycene, aliphatic poly(ether), aromatic polyether dendrimers, dendrimer of a sugar (e.g., glucose, galactose, mannose, fructose, etc.), and copolymers thereof, such as a copolymer of a sugar and an alkylene glycol (e.g., a dendrimer formed by glucose and ethylene glycol building blocks). The dendrimers can have a plurality of surface functional groups, such as carboxylic, amine, hydroxyl, and/or acetamide. The terms “surface functional groups” and “terminal groups” are used interchangeably herein. In some embodiments, the dendrimers have surface hydroxyl groups. In some embodiments, one or more of these surface functional groups are further modified with other molecules, such as further modified with a sugar (e.g., glucose, galactose, mannose, fructose, etc.) and/or a polyalkylene glycol, for example, polyethylene glycol, and thus have sugar molecules and/or polyalkylene glycols as terminal moieties/molecules. Dendrimers can be any generation including, but not limited to, generation 1, generation 2, generation 3, generation 4, generation 5, generation 6, generation 7, generation 8, generation 9, or generation 10. In some embodiments, the dendrimers are PAMAM dendrimers used as a platform and modified with functional groups for increased number of surface hydroxyl groups. Preferred PAMAM dendrimers include hydroxylated PAMAM dendrimers, particularly G3 to G6 hydroxyl-terminated PAMAM dendrimers, such as G3, G4, G5, and G6 hydroxyl-terminated PAMAM dendrimers.

In some embodiments, the dendrimer-active agent conjugates can be confined to the peripheral circulation and specifically target a particular tissue region and/or cell type, such as peripheral neural cells, glial cells and/or their supporting cells e.g., enteric neurons and glia, by using higher generation dendrimer (such as generation 4, 5, or 6 PAMAM dendrimer, generation 2, 3, or higher glucose-based dendrimers). Additionally, or alternatively, the dendrimer-active agent conjugates can be confined to the peripheral circulation by appropriate functionalization of the dendrimer (such as PEGylation).

In some embodiments, the dendrimers can specifically target a particular tissue region and/or cell type of the central nervous system (CNS), the peripheral nervous system (PNS), and/or the periphery, such as neurons and glia of the CNS, and/or neurons and glia of the PNS by using dendrimers of a certain generation, such as PAMAM dendrimers and/or glucose dendrimers of generation 2 (G2), G3, G4, and G5.

### Monosaccharide-Based Dendrimers

In preferred embodiments, the branching units include monosaccharides. In some embodiments, the monosaccharide branching units are conjugated to the core or the prior layer of monomers via linkers such as polyethylene glycol chains. In preferred embodiments, the monosaccharide branching units are glucose-based branching units. In some embodiments, the branching units can include PEG and/or alkyl chain linkers between different dendrimer generations. For example, the glucose layers are connected via PEG linkers and triazole rings. In some embodiments, the branching units are the same for each generation of dendrimers generated from the core. Therefore, for example, the branching units are glucose-based branching units for generating generation 1 dendrimers, for generating generation 2 dendrimers, and for generating generation 3 dendrimers.

In some embodiments, the dendrimers have a hypercore such as dipentaerythritol and one or more monosaccharide branching units. In some embodiments, the hypercore is dipentaerythritol and the monosaccharide branching unit is glucose-based branching unit. In further embodiments, spacer molecules can also be alkyl (CH2)n-hydrocarbon-like units.

In some embodiments, dendrimers synthesized using glucose building blocks, with a surface made predominantly of glucose moieties, specifically targets cells including injured neurons, ganglion cells, and other neuronal cells in the brain, the eye, and/or in peripheral nervous system. In some embodiments, the glucose-based dendrimer selectively targets or is enriched inside target neural and/or glial cells. In some embodiments, the glucose-based dendrimer selectively targets or enriches the surface of target neural and/or glial cells. In some embodiments, the glucose-based dendrimer selectively targets or is enriched inside target neuronal cells and on the surface of the target neural and/or glial cells. In some embodiments, the glucose-based dendrimer selectively targets or is enriched inside and/or on the surface of injured, diseased, and/or hyperactive neurons and/or glial cells.

In some cases, the dendrimers include an effective number of sugar molecules and terminal groups, for example, glucose and/or hydroxyl groups, for targeting to one or more neurons and/or glia of the CNS, PNS, and/or the eye. The terminal hydroxyl groups of these dendrimers may be part of terminal glucose molecules or extra hydroxyl groups that are not part of the glucose molecules, or a combination thereof. In some embodiments, all the terminal hydroxyl groups are part of the terminal glucose molecules. In some embodiments, the number of sugar molecules on the termination of dendrimer is determined by the generation number.

In some embodiments, dendrimers are made of glucose and oligoethylene glycol building blocks. Exemplary glucose dendrimers are shown in Structures V and VII.

Some exemplary glucose dendrimers include a generation 1 glucose dendrimer having 24 hydroxyl (—OH) end groups, a generation 2 glucose dendrimer having 96 hydroxyl (—OH) end groups, a generation 3 glucose dendrimer having 396 hydroxyl (—OH) end groups, and generation 4 glucose dendrimer having 1584 hydroxyl (—OH) end groups. For example, the glucose dendrimer is a generation 2 glucose-based dendrimer that has 24 glucose molecules at the periphery and 6 embedded glucose molecules in the backbone held together by PEG segments.

Dendrimer compositions that can selectively accumulate inside neurons, particularly in the nucleus of injured and/or hyperactive neurons, referred to as “glucose dendrimers” also accumulate at a high level inside activated microglia. However, compared to hydroxyl dendrimers which primarily accumulate in microglia, these dendrimers primarily go to neurons. Glucose dendrimers are described in U.S. Ser. No. 63/327,610 “Dendrimer Compositions for Targeted Delivery of Therapeutics to Neurons” by The Johns Hopkins University, inventors Kannan Rangaramanujam, Rishi Sharma, Anjali Sharma, Sujatha Kannan, Nirnath Sah, Mira Sachdeva, and Siva P. Kambhampati filed Apr. 5, 2022.

Glucose dendrimers include (a) a central core, (b) one or more branching units, wherein the branching units are monosaccharide glucose-based branching units, optionally with a linker conjugated thereto; and optionally (c) one or more therapeutic, prophylactic and/or diagnostic agents. Generally, the one or more branching units are conjugated to the central core, and the surface groups of the dendrimer are monosaccharide glucose molecules. In some embodiments, the central core is dipentaerythritol, or a hexa-propargylated derivative thereof. In some embodiments, the branching unit is conjugated to the central core via a linker such as a hydrocarbon or an oligoethylene glycol chain. In a preferred embodiment, the branching units are β-D-Glucopyranoside tetraethylene glycol azide having the following structure.

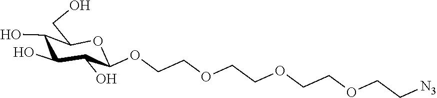

or peracetylated derivatives thereof.

In some embodiments, the glucose dendrimer is a generation 1, generation 2, generation 3, generation 4, generation 5, or generation 6 dendrimer. In one embodiment, the dendrimer is a generation 1 dendrimer having the following structure:

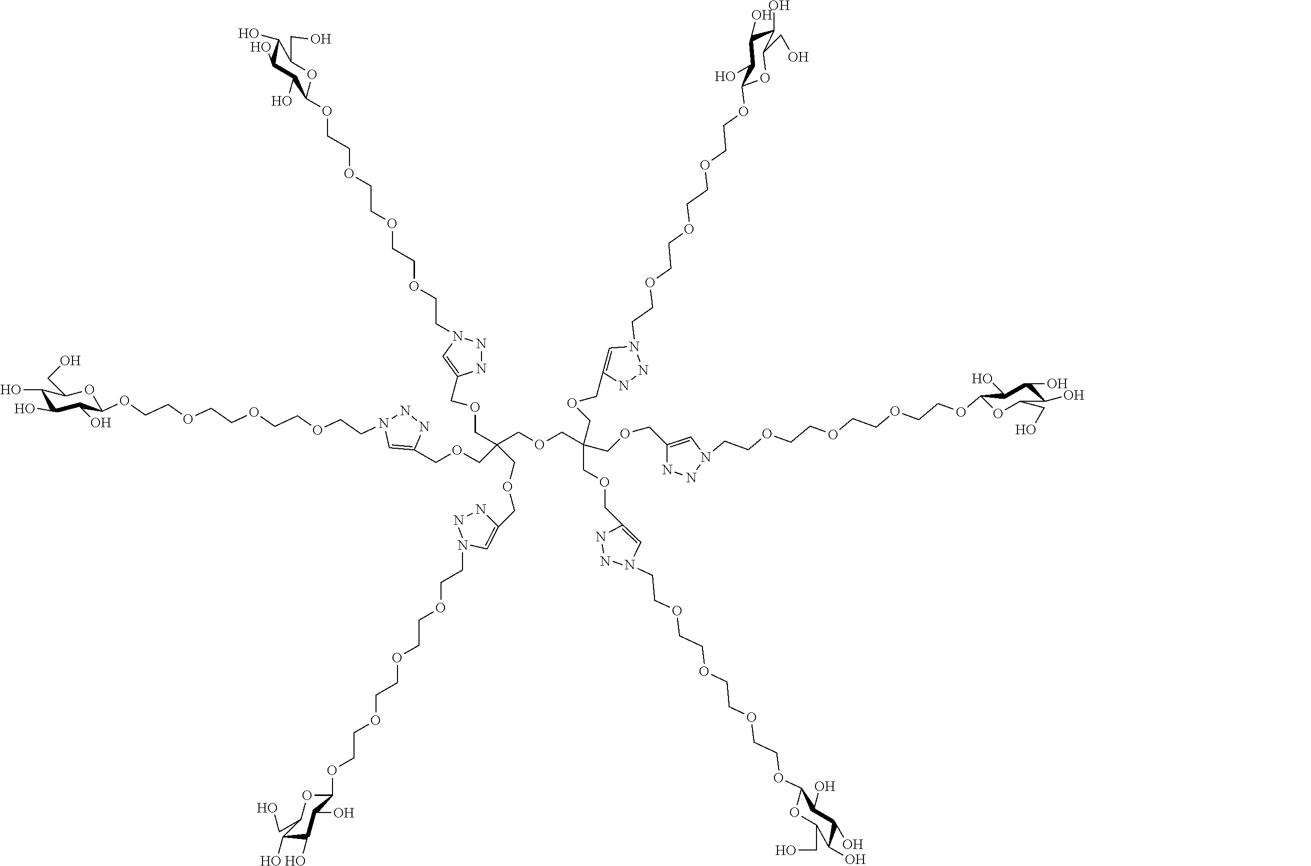

In a preferred embodiment, the dendrimer is a generation 2 dendrimer having the following structure:

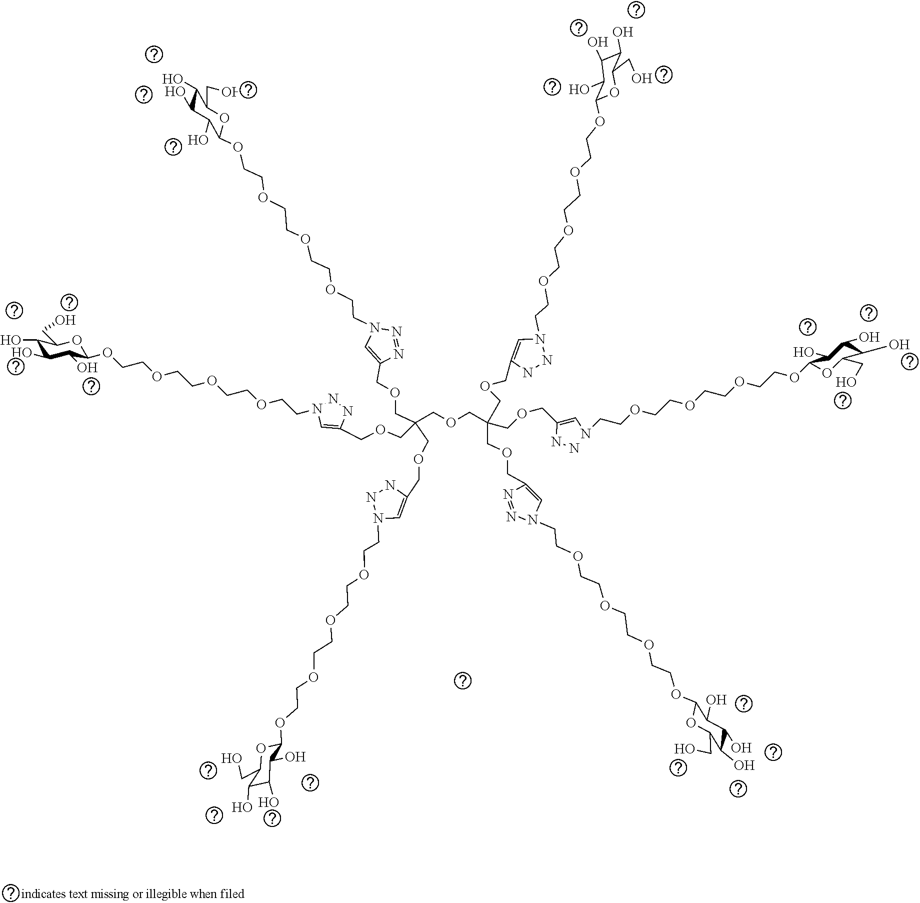

In some embodiments, the one or more therapeutic agents, prophylactic agents, and/or diagnostic agents are encapsulated, associated, and/or conjugated in the dendrimer, at a concentration of between about 0.01% to about 30%, preferably about 1% to about 20%, more preferably about 5% to about 20% by weight. The dendrimers may also be conjugated to one or more diagnostic agents such as fluorescent dyes, near infra-red dyes, SPECT imaging agents, PET imaging agents, and radioisotopes.

In some embodiments, the dendrimer and the agent(s) are conjugated via one or more linkers or coupling agents such as one or more hydrocarbon or oligoethylene glycol chains. Exemplary linkages are disulfide, ester, ether, thioester, and amide linkages.

### 1. Core

In some embodiments, dendrimers are prepared using methods in which the dendrimer is assembled from a multifunctional core, which is extended outward by a series of reactions. A multifunctional core moiety allows stepwise addition of branching units (i.e., generations) around the core

Exemplary chemical structures suitable as core moieties include dipentaerythritol, pentaerythritol, 2-(aminomethyl)-2-(hydroxymethyl) propane-1,3-diol, 2-ethyl-2-(hydroxymethyl) propane-1,3-diol, 3,3′,3″,3′-silanetetrayltetrakis (propane-1-thiol), 3,3-divinylpenta-1,4-diene, 3,3′,3″-nitrilotripropionic acid, 3,3′,3″-nitrilotris (N-(2-aminoethyl) propanamide), 3,3′,3″,3″-(ethane-1,2-diylbis(azanetriyl)) tetrapropanamide, 3-(carboxymethyl)-3-hydroxypentanedioic acid, 2,2′-((2,2-bis ((2-hydroxyethoxy)methyl) propane-1,3-diyl) bis(oxy))bis(ethan-1-ol), tetrakis (3-(trichlorosilyl) propyl) silane, 1-Thioglycerol, 2,2,4,4,6,6-hexachloro-1,3,5,215,415,615-triazatriphosphinine, 3-(hydroxymethyl)-5,5-dimethylhexane-2,4-diol, 4,4′,4″-(ethane-1,1,1-triyl) triphenol, 2,4,6-trichloro-1,3,5-triazine, 5-(hydroxymethyl)benzene-1,2,3-triol, 5-(hydroxymethyl)benzene-1,3-diol, 1,3,5-tris (dimethyl (vinyl) silyl)benzene, Carbosiloxane core, nitrilotrimethanol, ethylene diamine, propane-1,3-diamine, butane-1,4-diamine, 2,2′,2″-nitrilotris (ethan-1-ol), alpha cyclodextrin, beta cyclodextrin, gamma cyclodextrin, Cucurbituril, benzene-1,2,3,4,5,6-hexathiol, monosaccharide, disaccharides, trisaccharides, oligosaccharides, or azide-, alkyne-modified moieties thereof. In some embodiments, the core moiety is chitosan. Thus, azide-modified chitosan, or alkyne-modified chitosan are suitable for conjugating to branching units using click chemistry. In a preferred embodiment, the central core is dipentaerythritol or a hexa-propargylated derivative thereof.

In some embodiments, the core moiety is ethylenediamine, or tetra (ethylene oxide). In some embodiments, the core moiety is dipentaerythritol. Exemplary chemical structures suitable for use as core moieties are shown in Table 1 below.

TABLE 1
Structural representation of various building blocks (cores,
branching units, surface functional groups, monomers) for the synthesis of
dendrimers.

| Building Blocks | Structure |
|---|---|
| Dipentaerythritol | 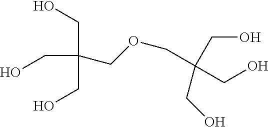 [SMILES: OCC(CO)(CO)COCC(CO)(CO)CO] |
| Pentaerythritol | 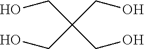 |
| 2-(aminomethyl)-2- (hydroxymethyl) propane-1,3-diol | 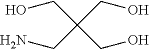 |
| 2-ethyl-2-(hydroxymethyl) propane-1,3-diol | 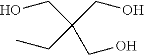 |
| 3,3′,3″,3′-silanetetrayltetrakis (propane-1-thiol) | 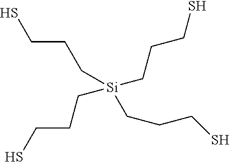 [SMILES: SCCC[Si](CCCS)(CCCS)CCCS] |
| 3,3-divinylpenta-1,4-diene | 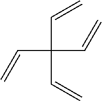 |
| 3,3′,3″-nitrilotripropionic acid | 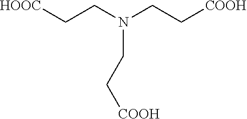 [SMILES: O=C(O)CCN(CCC(=O)O)CCC(=O)O] |
| 3,3′,3″-nitrilotris(N-(2- aminoethyl)propanamide) | 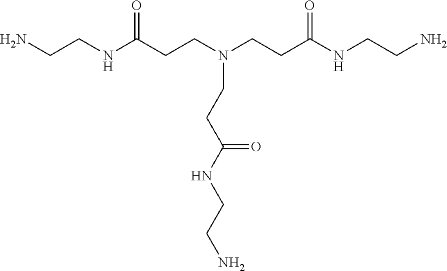 [SMILES: NCCNC(=O)CCN(CCC(=O)NCCN)CCC(=O)NCCN] |
| 3,3′,3″,3''-(ethane-1,2- diylbis(azanetriyl)) tetrapropanamide | 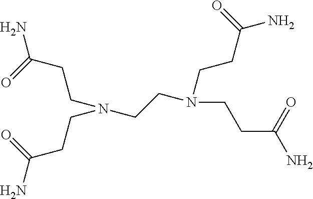 [SMILES: NC(=O)CCN(CCC(N)=O)CCN(CCC(N)=O)CCC(N)=O] |
| 3-(carboxymethyl)-3- hydroxypentanedioic acid | 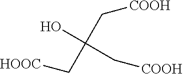 [SMILES: O=C(O)CC(O)(CC(=O)O)CC(=O)O] |
| 2,2′-((2,2-bis((2- hydroxyethoxy)methyl) propane-1,3- diyl)bis(oxy))bis(ethan-1-ol) | 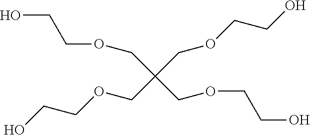 [SMILES: OCCOCC(COCCO)(COCCO)COCCO] |
| tetrakis(3-(trichlorosilyl) propyl)silane | 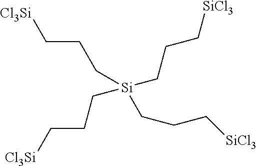 |
| 1-Thioglycerol | 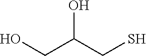 |
| All the sugar based scaffolds including mono, di, tri, or oligomers | 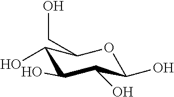 |
| 2,2,4,4,6,6-hexachloro-1,3,5,2l5, 4l5,6l5-triazatriphosphinine | 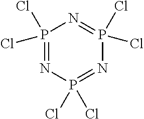 [SMILES: ClP1(Cl)=NP(Cl)(Cl)=NP(Cl)(Cl)=N1] |
| 3-(hydroxymethyl)-5,5- dimethylhexane-2,4-diol | 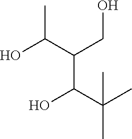 |
| 4,4′,4″-(ethane-1,1,1- triyl)triphenol | 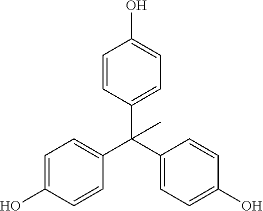 [SMILES: CC(c1ccc(O)cc1)(c1ccc(O)cc1)c1ccc(O)cc1] |
| 2,4,6-trichloro-1,3,5-triazine | 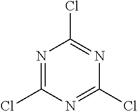 |
| 5-(hydroxymethyl) benzene-1,2,3-triol | 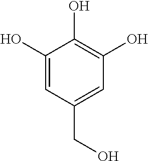 [SMILES: OCc1cc(O)c(O)c(O)c1] |
| 5-(hydroxymethyl)benzene-1,3- diol | 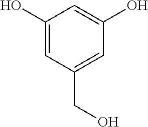 |
| 1,3,5- tris(dimethyl(vinyl)silyl)benzene | 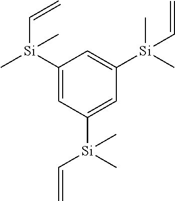 [SMILES: C=C[Si](C)(C)c1cc([Si](C)(C)C=C)cc([Si](C)(C)C=C)c1] |
| Carbosiloxane core | 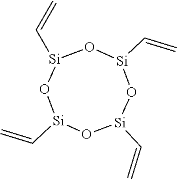 |
| nitrilotrimethanol | 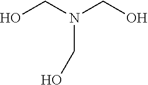 |
| ethylene diamine | 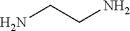 |
| propane-1,3-diamine | 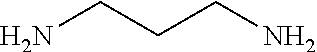 |
| butane-1,4-diamine | 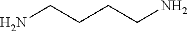 |
| 2,2′,2″-nitrilotris(ethan-1-ol) | 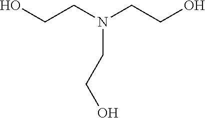 |
| | 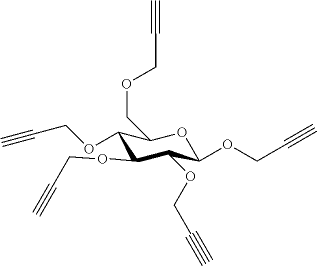 |
| | 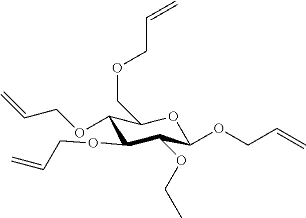 |
| | 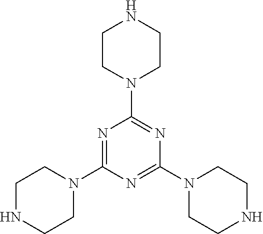 [SMILES: C1CN(c2nc(N3CCNCC3)nc(N3CCNCC3)n2)CCN1] |
| benzene-1,2,3,4,5,6-hexathiol | 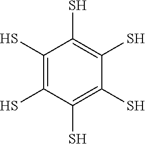 [SMILES: Sc1c(S)c(S)c(S)c(S)c1S] |
| alpha cyclodextrin | 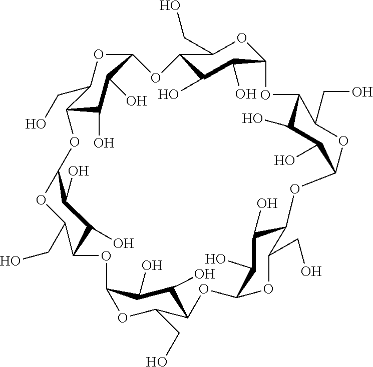 [SMILES: OCC1O[C@@H]2O[C@@H]3C(CO)O[C@H](O[C@@H]4C(CO)O[C@H](O[C@@H]5C(CO)O[C@H](O[C@@H]6C(CO)O[C@H](O[C@@H]7C(CO)O[C@H](O[C@H]1[C@H](O)C2O)C(O)[C@H]7O)C(O)[C@H]6O)C(O)[C@H]5O)C(O)[C@H]4O)C(O)[C@H]3O] |
| beta cyclodextrin | 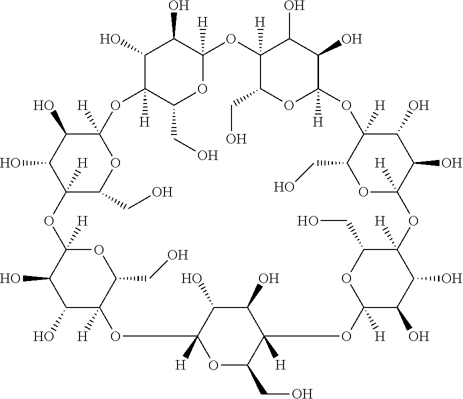 [SMILES: OC[C@H]1O[C@@H]2O[C@H]3C(O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO] |
| gamma cyclodextrin | 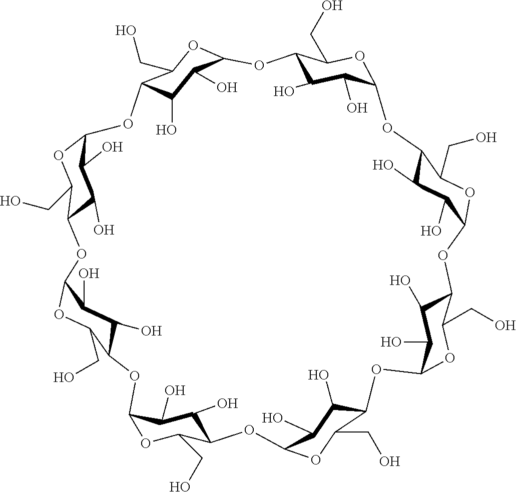 [SMILES: OCC1O[C@@H]2O[C@@H]3C(CO)O[C@H](O[C@@H]4C(CO)O[C@H](O[C@@H]5C(CO)O[C@H](O[C@@H]6C(CO)O[C@@H](O[C@@H]7C(CO)O[C@H](O[C@@H]8C(CO)O[C@H](O[C@@H]9C(CO)O[C@H](O[C@H]1[C@H](O)C2O)C(O)[C@H]9O)C(O)[C@H]8O)C(O)[C@H]7O)C(O)[C@H]6O)C(O)[C@H]5O)C(O)[C@H]4O)C(O)[C@H]3O] |
| Cucurbituril | 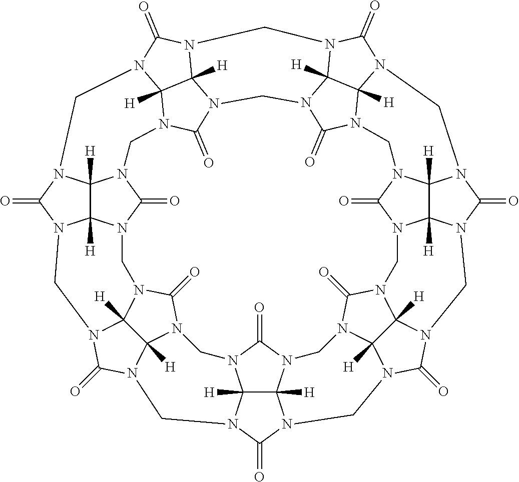 |

### 2. Branching Units

Exemplary chemical structures suitable as branching units include monosaccharides. In some embodiments, the monosaccharide branching units are conjugated to the core or the prior layer of monomers via linkers such as polyethylene glycol chains. In preferred embodiments, the monosaccharide branching units are glucose-based branching units. Exemplary glucose-based branching units are shown in Structures II-IV. These are spacer molecules, so can also be alkyl (CH₂) n-hydrocarbon-like units.

The branching units are the PEG or alkyl chain linkers between different dendrimer generations, for example, the glucose layers are connected via PEG linkers and triazole rings.

In preferred embodiments, the branching units are the same for each generation of dendrimers generated from the core. Therefore, in one embodiment, the branching units are glucose-based branching units for generating generation 1 dendrimers as shown in Structures V-VII.

In some embodiments, the branching units are hyper-monomers i.e., ABn building blocks. Exemplary hyper-monomers include AB4, AB5, AB6, AB7, AB8 building blocks. Hyper-monomer strategy drastically increases the number of available end groups. An exemplary AB4 hypermonomer is peracetylated β-D-Glucopyranoside tetraethylene glycol azide as shown in Structure III.

The chemical structures listed in Table 1, are also suitable as building blocks to form the branching units of the dendrimer. For example, the branching units of the dendrimers are formed by dipentaerythritol, pentaerythritol, 2-(aminomethyl)-2-(hydroxymethyl) propane-1,3-diol, 2-ethyl-2-(hydroxymethyl) propane-1,3-diol, 3,3′,3″,3″-silanetetrayltetrakis (propane-1-thiol), 3,3-divinylpenta-1,4-diene, 3,3′,3″-nitrilotripropionic acid, 3,3′,3″-nitrilotris (N-(2-aminoethyl) propanamide), 3,3′,3″,3′-(ethane-1,2-diylbis(azanetriyl)) tetrapropanamide, 3-(carboxymethyl)-3-hydroxypentanedioic acid, 2,2′-((2,2-bis ((2-hydroxyethoxy)methyl) propane-1,3-diyl) bis(oxy))bis(ethan-1-ol), tetrakis (3-(trichlorosilyl) propyl) silane, 1-Thioglycerol, 2,2,4,4,6,6-hexachloro-1,3,5,215,415,615-triazatriphosphinine, 3-(hydroxymethyl)-5,5-dimethylhexane-2,4-diol, 4,4′,4″-(ethane-1,1,1-triyl) triphenol, 2,4,6-trichloro-1,3,5-triazine, 5-(hydroxymethyl)benzene-1,2,3-triol, 5-(hydroxymethyl)benzene-1,3-diol, 1,3,5-tris (dimethyl (vinyl) silyl)benzene, Carbosiloxane core, nitrilotrimethanol, ethylene diamine, propane-1,3-diamine, butane-1,4-diamine, 2,2′,2″-nitrilotris (ethan-1-ol), alpha cyclodextrin, beta cyclodextrin, gamma cyclodextrin, Cucurbituril, benzene-1,2,3,4,5,6-hexathiol, monosaccharide, disaccharides, trisaccharides, oligosaccharides, or azide-, alkyne-modified moieties thereof, or a combination thereof. Other examples of chemical structures that are suitable for forming the branching units of the dendrimers include, but are not limited to, sugar moieties, such as glucose, galactose, mannose, and fructose, and alkylene glycol, such as ethylene glycol, and combinations thereof. In some embodiments, the branching unit is chitosan. Thus, azide-modified chitosan, or alkyne-modified chitosan are suitable for conjugating to the core moiety or additional same or different branching units using click chemistry. In some embodiments, the branching unit is methyl acrylate or ethylenediamine, or a combination thereof. In some embodiments, the branching unit is polyethylene glycerol linear or branched. In some embodiments, the branching unit is a copolymer of an alkylene glycol (such as ethylene glycol) and a sugar moiety, such as glucose, galactose, mannose, and/or fructose.

### 3. Surface Functional Groups

Surface functional groups/molecules of the dendrimers are not limited to a primary amine end group, a hydroxyl end group, a carboxylic acid end group, an acetamide end group, a sugar molecule, an oligo- or poly-alkylene glycol, and/or a thiol end group. In some embodiments, the desired terminal functional groups can be added via one of the conjugation methods for the core and branching unit.

In some embodiments, the surface functional groups are hydroxyl groups, for example those of PAMAM dendrimers, of generation 2 OEG dendrimer as shown in Structure I, or of the terminal glucose of dendrimers prepared with glucose-based branching units as shown in Structures V and VII. In some embodiments, desired surface functional groups can be modified or added via one of the conjugation methods for the core and branching unit. Exemplary surface functional groups include hydroxyl end groups, amine end groups, carboxylic acid end groups, acetamide end group, and thiol end groups, and combinations thereof.

In some embodiments, the dendrimers can specifically target a particular tissue region and/or cell type, such as the cells and tissues of the central nervous system (CNS), the peripheral nervous system (PNS), and/or the eye. In some embodiments, the dendrimers specifically target neurons and/or glia of the CNS. In some embodiments, the dendrimers specifically target neurons and/or glia of the PNS. In some embodiments, the dendrimers specifically target non-neural and/or non-glial cells such as gastrointestinal cells, cardiovascular cells, and/or immune system cells. In some embodiments, the glucose dendrimers are those of generation 1 (G1), G2, G3, G4, and G5, preferably G1, G2, and/or G3.

In some embodiments, the dendrimers include an effective number of terminal glucose and/or hydroxyl groups for targeting to one or more neurons and/or glia of the CNS, the PNS, and/or the eye. In some embodiments, the dendrimers include an effective number of terminal glucose and/or hydroxyl groups for targeting to one or more non-neural and/or non-glial cells such as gastrointestinal cells, cardiovascular cells, and/or immune system cells.

In some embodiments, dendrimers are made of glucose and oligoethylene glycol building blocks. Exemplary generation 1 glucose dendrimer is shown in Structure VI, and generation 2 glucose dendrimers is shown in Structure VIII.

In some embodiments, the dendrimers have a plurality of surface functional groups, such as hydroxyl (—OH) groups, amine groups, acetamide groups, and/or carboxyl groups on the periphery of the dendrimers (also referred to herein as surface functional groups or peripheral functional groups). In some embodiments, the surface density of such peripheral functional groups is at least 1 group/nm² (number of the surface functional groups/surface area in nm²). For example, in some embodiments, the surface density of the surface functional groups, such as hydroxyl groups, is more than 0.5, 1, 2, 3, 4, 5, 6, 7, 8, 9, 10 OH groups/nm², such as at least 10, 15, 20, 25, 30, 35, 40, 45, 50, or more than 50 OH groups/nm². In some embodiments, the volumetric density of surface functional groups, such as hydroxyl groups, is between about 1 and about 50 groups/nm³, between about 5 and about 30 groups/nm³, or between about 10 and about 20 groups/nm³. In further embodiments, the surface density of the surface functional groups, such as hydroxyl groups, is between about 1 and about 50, preferably 5-20 group/nm² (number of surface functional groups/surface area in nm²), while each surface functional moiety has a molecular weight of between about 100 Da and about 10 kDa, preferably between about 100 Da and 1000 Da.

In some embodiments, the amount of the surface functional groups, such as any one of those described above, e.g., hydroxyl groups, of the dendrimer is at least 30%, at least 40%, at least 50%, more than 40%, more than 50%, or in a range from more than 30% to 100%. %. In preferred embodiments, the amount of surface hydroxyl groups of the dendrimer is preferably more than 35%.

In some embodiments, one or more of the surface functional groups, such as any one of those described above, on the periphery of the dendrimers are further modified by conjugating with one or more carbohydrate molecules and/or more or more polyalkylene glycols, such as polyethylene glycols. In these embodiments, the surface density of the terminal carbohydrate moieties/molecules and/or polyalkylene glycols can have any of the ranges described above for hydroxyl groups. Hydroxyl-terminated PAMAM dendrimers, PAMAM dendrimer modified on the surface with sugar moieties (with >10% of surface groups modified by sugars, especially by glucose, and glucose dendrimers (where the dendrimers are made of glucose building blocks are preferred). For delivery to the brain, constructs with a total molecular weight of <30,000 Da are preferred. For confinement primarily to the peripheral circulation, constructs with a total molecular weight of >50,000 Da are preferred. When dendrimers are formed of, or include sugar moieties/molecules at termination, for example, glucose, the terminal hydroxyl groups of these dendrimers may be part of the terminal sugar moieties/molecules or extra hydroxyl groups that are not part of the sugar moieties/molecules, or a combination thereof. In some embodiments, all of the terminal hydroxyl groups are part of the terminal sugar moieties/molecules.

a. Hydroxyl-Terminated Dendrimers

In some embodiments, the dendrimers include a plurality of hydroxyl groups. Some exemplary high-density hydroxyl groups-containing dendrimers include commercially available polyester dendritic polymer such as hyperbranched 2,2-Bis (hydroxyl-methyl) propionic acid polyester polymer (for example, hyperbranched bis-MPA polyester-64-hydroxyl, generation 4), dendritic polyglycerols. In some embodiments, the hydroxyl terminated dendrimers include hydroxyl-terminated PAMAM dendrimers, particularly G3 to G6 hydroxyl-terminated PAMAM dendrimers, such as G3, G4, G5, and G6 hydroxyl-terminated PAMAM dendrimers.

In some embodiments, the high-density hydroxyl groups-containing dendrimers are oligo ethylene glycol (OEG)-like dendrimers. For example, a generation 2 OEG dendrimer (D2-OH-60) as shown in Structure I can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in WO2019094952. In some embodiments, the dendrimer backbone has non-cleavable polyether bonds throughout the structure to avoid the disintegration of dendrimer in vivo and to allow the elimination of such dendrimers as a single entity from the body (non-biodegradable).

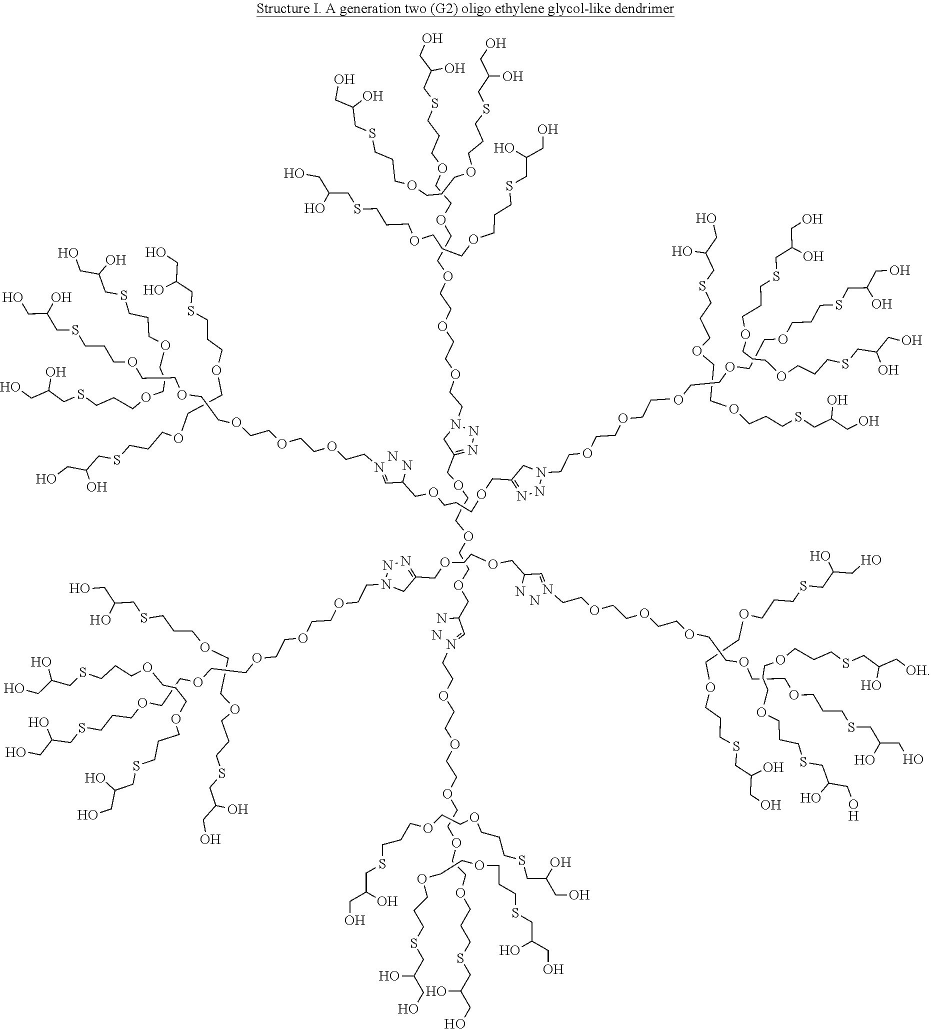

In some embodiments, the dendrimers have a plurality of hydroxyl (—OH) groups on the periphery of the dendrimers. In some embodiments, the surface density of hydroxyl (—OH) groups is at least 1 OH group/nm² (number of surface hydroxyl groups/surface area in nm²). For example, in some embodiments, the surface density of hydroxyl groups, per nm², is more than 0.5, 1, 2, 3, 4, 5, 6, 7, 8, 9, 10 OH groups/nm², such as at least 10, 15, 20, 25, 30, 35, 40, 45, 50, or more than 50 OH groups/nm². In some embodiments, the volumetric density of hydroxyl groups is between about 1 and about 50 groups/nm³, between about 5 and about 30 groups/nm³, or between about 10 and about 20 groups/nm³. In further embodiments, the surface density of hydroxyl (—OH) groups is between about 1 and about 50, or between 5 and 20 OH group/nm² (number of surface hydroxyl groups/surface area in nm²) while having a molecular weight of between about 100 Da and about 1000 Da. In some embodiments, the amount of the surface hydroxyl groups of the dendrimer is preferably greater than 35%, at least 40%, at least 50%, more than 40%, more than 50%, or in a range from more than 40% to 100. In some embodiments, the dendrimers may have a fraction of the hydroxyl groups exposed on the outer surface, with the others in the interior core of the dendrimers.

In some embodiments, the dendrimer specifically targets a particular tissue region and/or cell types following administration into the body. In some embodiments, the dendrimer specifically targets a particular tissue region and/or cell type without a targeting moiety. In some embodiments, the dendrimers include an effective number of hydroxyl groups for targeting CNS cells and/or PNS cells, such as microglial, astrocytes, and/or neurons associated with a disease, disorder, or injury of the central nervous system or the peripheral nervous system. In some embodiments, the dendrimer specifically targets a particular tissue region and/or cell type without a targeting moiety and the active agent conjugated thereto bind directly to a receptor on the surface and/or interior of target neural and/or glial cells. Unmodified PAMAM dendrimers with hydroxyl end groups do not enrich in the neurons of brain and/or retinal ganglion cells (RGCs) in the eye as much as the glucose dendrimers. The glucose dendrimers with terminal glucose monosaccharide and a high density of hydroxyl functional groups effectively target the neurons in a generation dependent manner. Generation 2 (G2), and G3 and G4 should be efficacious. G5 and above are more difficult to use.

In preferred embodiments, the dendrimers include an effective number of terminal glucose and/or hydroxyl groups for targeting to one or more neurons of the CNS, or the eye. The hydroxyl groups on the dendrimer surface are part of glucose molecules. There are no extra hydroxyls in addition to the glucose molecules on the surface. The number of sugar molecules on the surface is determined by the generation number. All generations are expected to target neurons.

Some exemplary glucose dendrimers include a generation 1 glucose dendrimer having 24 hydroxyl (—OH) end groups, a generation 2 glucose dendrimer having 96 hydroxyl (—OH) end groups, a generation 3 glucose dendrimer having 396 hydroxyl (—OH) end groups, and generation 4 glucose dendrimer having 1584 hydroxyl (—OH) end groups. In a preferred embodiment, the glucose dendrimer is a generation 2 glucose based dendrimer that has 24 glucose molecules at the periphery and 6 embedded glucose molecules in the backbone held together by PEG segments.

b. Dendrimers Modified with Carbohydrates

In some embodiments, the dendrimers contain one or more carbohydrate molecules at the termination. These terminal carbohydrate molecules can be prepared by conjugating one or more surface functional groups of a dendrimers, such as amine groups, carboxyl groups, or hydroxyl groups, with one or more carbohydrate molecules. In preferred embodiments, the dendrimers, prior to carbohydrate conjugation, are hydroxyl-terminated dendrimers such as hydroxyl-terminated PAMAM dendrimers and one or more of the hydroxyl groups are conjugated with one or more carbohydrate molecules.

In some embodiments, hydroxyl-terminated dendrimers modified with surface glucose molecules selectively target central and/or peripheral neural and/or glial cells in vitro and in vivo; and/or selectively accumulate on the surface and/or within these targets, such that the active agent(s) conjugated thereto bind to one or more receptors on/in the target neural and/or glial cells. In some embodiments, hydroxyl-terminated dendrimers modified with surface glucose molecules selectively target gastrointestinal cells, cardiovascular cells, and/or immune system cells in vitro and in vivo; and/or selectively accumulate on the surface and/or within these targets, such that the active agent(s) conjugated thereto bind to one or more receptors on/in the target gastrointestinal cells, cardiovascular cells, and/or immune system cells.

In some embodiments, the carbohydrate moieties used to modify one or more surface functional groups of the dendrimers are monosaccharides. Exemplary monosaccharides suitable for modifying the dendrimers include glucose, glucosamine, galactose, mannose, fructose, dehydroascorbic acid, urate, myo-inositol. In some embodiments, the dendrimers are conjugated to glucose and thus contain glucose as terminal moieties/molecules. In some embodiments, hydroxyl-terminated dendrimers are modified with one or more glucose moieties to the dendrimer (“D-Glu”). In some embodiments, the dendrimers are conjugated to galactose. In some embodiments, the dendrimers are conjugated to mannose. In some embodiments, the dendrimers are conjugated to fructose. In some embodiments, the dendrimers are conjugated to one or more monosaccharides other than glucose, such as galactose, mannose, and/or fructose. For example, the carbohydrate moieties are oligosaccharides which terminate in one or more monosaccharides including glucose, glucosamine, mannose, fructose, thus exposing these sugar moieties on the surface for binding.

In preferred embodiments, the glucose or hydroxyl-terminated PAMAM dendrimers, or carbohydrate-functionalized dendrimers, are conjugated to one or more active agents that have affinity to and are suitable for binding directly or indirectly, one or more of serotonin (5HT) receptors e.g., 5HT-1A, 5HT-2B, 5HT-2A, 5HT-2B, 5HT-2C, 5HT-3, 5HT-4, 5HT-6, and 5HT-7 receptors. In some embodiments, the dendrimers are conjugated to one or more carbohydrates moieties that have affinity to and are suitable for binding one or more norepinephrine (NE) receptors e.g., α_{2A}-adrenergic receptor, α_{2B}-adrenergic receptor, α_{2C}-adrenergic receptor, and/or β-adrenergic receptor. In some embodiments, the dendrimers are conjugated to one or more carbohydrates moieties that have affinity to and are suitable for binding directly or indirectly, dopamine D1 and D2 receptors. In some embodiments, the dendrimers are conjugated to one or more carbohydrates moieties that have affinity to and are suitable for binding one or more monoamine transporters e.g., vesicular monoamine transporter 2 (VMAT2), serotonin reuptake transporter (SERT), the noradrenaline transporter (NAT), the dopamine transporter (DAT). In some embodiments, the dendrimers are conjugated to one or more carbohydrates moieties that have affinity to and are suitable for binding one or more of AMPA receptors, NMDA receptors, EGFR1 receptors, EGFR2 receptors, histamine (H1) receptors, GABA receptors, and trace amine-associated receptor 1 (TAAR1). In some embodiments, the dendrimers, with or without carbohydrate moieties, are conjugated to one or more active agents that have affinity to and are suitable for transport via one or more of GLUT1, GLUT2, GLUT3, GLUT4, GLUT5, GLUT6, GLUT7, GLUT8, GLUT9, GLUT10, GLUT11, GLUT12, GLUT13, and GLUT14. In further embodiments, the dendrimers are conjugated to one or more glucose and/or glucosamine moieties. In some embodiments, the dendrimers contain carbohydrate moieties which enable transport of the active agent to target cells/receptors, wherein the activity at the target cell or receptor is driven by the active agent. In these embodiments, the carbohydrates and glucose moieties enable better drug targeting to cells and/or receptors of interest. For example, in some embodiments, the dendrimers are conjugated to one or more glucose and/or glucosamine moieties. In other embodiments, the dendrimers are conjugated to one or more oligosaccharides terminating in glucose and/or glucosamine moieties, i.e., glucose and/or glucosamine moieties are exposed on the surface of the dendrimer conjugates suitable for binding to one or more of the GLUTs, 5HT receptors, NE receptors, DA receptors and/or transporters.

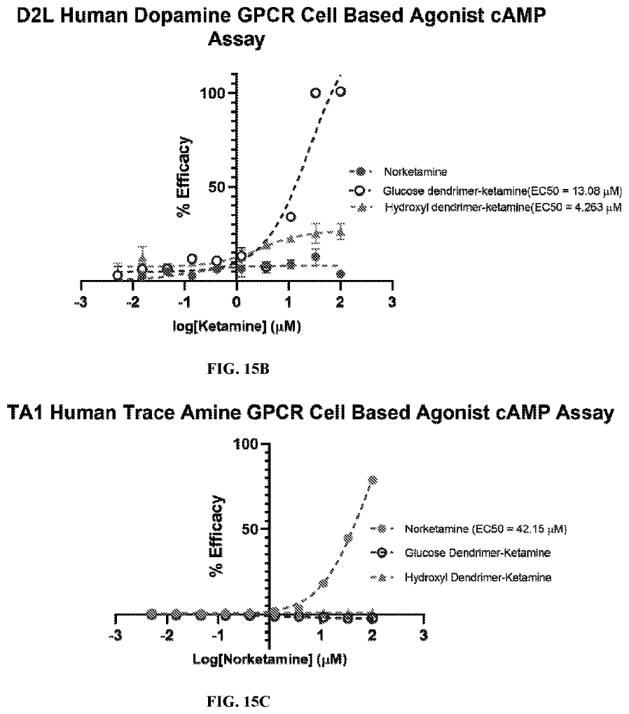

FIG. 13 is a schematic overview of the main pharmacological targets of LSD, psilocybin, DMT, MDMA, and ketamine, the signaling cascades involved, hormonal modulation, as well as main behavioral outcomes following their administration in both animals and humans.

In some embodiments, the dendrimers have a plurality of carbohydrate moieties/molecules such as monosaccharides, e.g., glucose, on the periphery of the dendrimers. In some embodiments, the surface density of carbohydrate molecules such as monosaccharides, e.g., glucose, is at least 1 carbohydrate molecule/nm² (number of surface carbohydrate groups/surface area in nm²). In some embodiments, the surface density of carbohydrate molecules, per nm², is more than 0.5, 1, 2, 3, 4, 5, 6, 7, 8, 9, 10 OH groups/nm², such as at least 10, 15, 20, 25, 30, 35, 40, 45, 50, or more than 50 OH groups/nm². For example, surface density of carbohydrate molecules, per nm², is more than 10. In some embodiments, the volumetric density of surface carbohydrate molecules is between about 1 and about 50 groups/nm³, between about 5 and about 30 groups/nm³, or between about 10 and about 20 groups/nm³. In further embodiments, the surface density of carbohydrate molecules is between about 1 and about 50, between about 5 and about 20, per nm² (number of surface carbohydrate molecules/surface area in nm²) while each carbohydrate moiety having a molecular weight of between about 100 Da and about 1000 Da. In these embodiments, i.e., one or more surface functional groups of the dendrimer are modified to introduce one or more sugar moieties/molecules at termination, the terminal hydroxyl groups may be part of the terminal sugar moieties/molecules or extra hydroxyl groups that are not modified with sugar moieties/molecules and thus are not part of the sugar moieties/molecules, or a combination thereof.

In some embodiments, carbohydrate molecules such as monosaccharides, e.g., glucose, are present in an amount by weight that is between about 1% and 40% of the total weight of the glycosylated dendrimer, for example, between about 2% and 20%, between about 5% and 15%, or between 9% and 12% of the total weight of the glycosylated dendrimer. For example, in some embodiments, the carbohydrate moieties are present in an amount that is about 1%, 2%, 3%, 4%, 5%, 6%, 7%, 8%, 9%, 10%, 11%, 12%, 13%, 14%, 15%, 16%, 17%, 18%, 19%, or 20% of the total weight of the glycosylated dendrimer following conjugation. In some embodiments, conjugation of carbohydrate molecules through one or more surface functional groups occurs via about 5%, 6%, 7%, 8%, 9%, 10%, 15%, 20%, or 25% of the total available surface functional groups, preferably hydroxyl groups, of the dendrimers prior to the conjugation. In other embodiments, the conjugation of carbohydrate molecules occurs on less than 5%, less than 10%, less than 15%, less than 20%, less than 25%, less than 30%, less than 35%, less than 40% of total available surface functional groups of the dendrimers prior to the conjugation.

c. Dendrimers Modified with Polyalkylene Glycol

In some embodiments, the dendrimers contain one or more polyalkylene glycols at the termination. These terminal polyalkylene glycols can be prepared by conjugating one or more of surface functional groups of the dendrimers, such as hydroxyl groups, with a polyalkylene glycol, such as PEG. In some embodiments, the dendrimers, prior to conjugation, are hydroxyl-terminated dendrimers such as hydroxyl-terminated PAMAM dendrimers and at least a portion of the surface hydroxyl groups are conjugated with PEG.

In some embodiments, the dendrimers have a plurality of polyalkylene glycols such as PEG, on the periphery of the dendrimers. In some embodiments, the surface density of polyalkylene glycols such as PEG, is at least 1 polyalkylene glycol/nm² (number of surface polyalkylene glycol/surface area in nm²). In some embodiments, the surface density of polyalkylene glycols, per nm², is more than 0.5, 1, 2, 3, 4, 5, 6, 7, 8, 9, 10 polyalkylene glycol/nm², such as at least 10, 15, 20, 25, 30, 35, 40, 45, 50, or more than 50 polyalkylene glycol/nm². For example, surface density of polyalkylene glycols, per nm², is more than 10. In some embodiments, the volumetric density of surface polyalkylene glycols is between about 1 and about 50 groups/nm³, between about 5 and about 30 groups/nm³, or between about 10 and about 20 groups/nm³. In further embodiments, the surface density of polyalkylene glycols such as PEG is between about 1 and about 50, between about 5 and about 20, per nm² (number of surface polyalkylene glycols/surface area in nm²) while having a molecular weight of between about 100 Da and about 1000 Da.

In some embodiments, the polyalkylene glycol molecules such as PEG can be present in an amount by weight that is between about 1% and 40% of the total weight of the pegylated dendrimer, for example, between about 2% and 20%, between about 5% and 15%, or between 9% and 12% of the total weight of the pegylated dendrimer. For example, in some embodiments, the polyalkylene glycol molecules, such as PEG, are present in an amount that is about 1%, 2%, 3%, 4%, 5%, 6%, 7%, 8%, 9%, 10%, 11%, 12%, 13%, 14%, 15%, 16%, 17%, 18%, 19%, or 20% of the total weight of the pegylated dendrimer following conjugation.

In some embodiments, conjugation of polyalkylene glycol molecules such as PEG through one or more surface functional groups of the dendrimer occurs via about 5%, 6%, 7%, 8%, 9%, 10%, 15%, 20%, or 25% of the total available surface functional groups, preferably hydroxyl groups, of the dendrimers prior to the conjugation. In other embodiments, the conjugation of polyalkylene glycol molecules such as PEG occurs on less than 5%, less than 10%, less than 15%, less than 20%, less than 25%, less than 30%, less than 35%, less than 40% of total available surface functional groups of the dendrimers prior to the conjugation.

### B. Active Agents

The dendrimers are conjugated or complexed with one or more hallucinogens. Hallucinogens are a diverse group of drugs that alter a subject's awareness of their surroundings as well as their own thoughts and feelings. Collectively referred to as “psychedelics”, hallucinogens can be split into two categories: classic hallucinogens (such as LSD) and dissociative drugs (such as PCP). Both types of hallucinogens can cause hallucinations, or sensations and images that seem real though they are not. Additionally, dissociative drugs can cause subjects to feel out of control or disconnected from their body and environment. Exemplary hallucinogens that can be conjugated to the dendrimer compositions include but are not limited to a range of drug classes including but not limited to classical psychedelics, dissociatives, and deliriants. The hallucinogens and their derivatives typically bind to one or more receptors, thereby modulating signaling in neurotransmitter signaling in the central nervous system and peripheral nervous system. Hallucinogens typically inhibit reuptake of neurotransmitters, particularly serotonin, dopamine, and noradrenaline, through selective receptors thereby increasing the concentration of these specific neurotransmitters in the synaptic cleft. Thus, partial antagonism, functional selectivity and inverse agonism all play important roles in determining the cellular response to specific neurotransmitter receptor ligands.

In preferred embodiments, the dendrimers are conjugated to one or more psychedelic hallucinogens such as psilocin, ketamine (R-ketamine, S-ketamine, (R/S)-ketamine), norketamine, ketamine analogues, ketamine metabolites, N,N dimethyl tryptamine (DMT), 4-acetoxy-N,N-dimethyltryptamine, 5-methoxy DMT, 5-chloro DMT, LSD, 3,4-methylenedioxymethamphetamine (MDMA), psilocybin, ibogaine, mescaline, norbaeocystin, 2C compounds (synthetic psychedelic drugs which belong to a group of designer agents similar in structure to Ecstasy and MDMA), NBOMes (N-benzylmethoxy derivatives of the 2C family hallucinogens-4-Iodo-2,5-dimethoxy-N-(2-methoxybenzyl) phenethylamine (25I-NBOMe) exhibits high binding affinity for 5-HT2A/C and 5-HT_{1A} serotonin receptors, 3,4-methylenedioxyethylamphetamine (MDE), d-lysergic acid diethylamide (LSD), or their analogues. In some embodiments, the psychedelic hallucinogens functionalized, for example with ester, disulfide, phosphodiester, triglycyl peptide, hydrazine, amide, ether, and amino alkyl linkage, optionally with one or more spacers/linkers, for ease of conjugation with the dendrimers and/or for desired release kinetics.

Most of the psychedelics have poor aqueous solubility in the ug/mL range. Therefore, in some embodiments, dendrimer conjugation of these psychedelics improves the water solubility between about 2-fold and about 200 fold, inclusive; between about 5-fold and about 150-fold, inclusive; between about 10-fold and about 100-fold, inclusive, compared to the free drug, i.e., without conjugation to a dendrimer.

### 1. Classical Psychedelics

The compositions may include a dendrimer complexed to one or more classical psychedelics. Classic or serotoninergic psychedelic compounds are so called mainly because they interact with the serotonergic system and most of them derive from plants or are semisynthetic compounds. In some cases, the classical psychedelics share part of the chemical structure with the endogenous neurotransmitter serotonin (5-HT)-in particular, the indole scaffold. However, some of them, like mescaline, do not possess an indole but are still considered serotonergic psychedelics. Typically, the classical psychedelics exert their effects via serotonin 2A receptor (5HT-2A) agonism.

The classical psychedelic conjugated to the dendrimer composition may be one or more semisynthetic ergoline LSD, plant-derived tryptamines, and/or phenethylamines. Exemplary serotonergic hallucinogens include indolamines, such as psilocybin and LSD, and phenylethylamines, such as mescaline and 2,5-dimethoxy-4-iodoamphetamine (DOI).

a. Tryptamines

The classical psychedelic conjugated to the dendrimer composition may be one or more tryptamines. Tryptamine is an indolamine metabolite of the essential amino acid, tryptophan. The chemical structure is defined by an indole—a fused benzene and pyrrole ring, and a 2-aminoethyl group at the second carbon (third aromatic atom, with the first one being the heterocyclic nitrogen). Tryptamine activates trace amine-associated receptors expressed in the brain, and regulates the activity of dopaminergic, serotonergic, and glutamatergic systems. In the gut, symbiotic bacteria convert dietary tryptophan to tryptamine, which activates 5-HT4 receptors and regulates gastrointestinal motility.

Exemplary psychedelic tryptamines that may be conjugated to the dendrimer include DMT, etryptamine, N,N-diethyltryptamine (DET), Psilocin and psilocybin, and their derivatives.

i. Psilocybin

Psilocybin is a high-affinity agonist at serotonin 5-HT2A receptors, which are especially prominent in the prefrontal cortex. It increases cortical activity secondary to down-stream post-synaptic glutamate effects. It is also active at 5-HT_{1A}, 5-HT_{1D}, and 5-HT_{2C} receptors, although these are thought to play a lesser role in its effects. Psilocybin is well tolerated and safe for human studies at oral doses of 8-25 mg and intravenous doses of 1-2 mg.

### Structure of Psilocybin

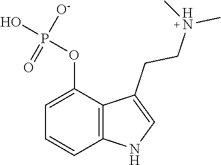

i. N,N-Dimethyltryptamine (DMT)

N,N-Dimethyltryptamine (DMT or N,N-DMT) is a substituted tryptamine and which is both a derivative and a structural analog of tryptamine. DMT has a rapid onset, intense effects, and a relatively short duration of action. DMT binds to the following serotonin receptors: 5-HT1A, 5-HT1B, 5-HTID, 5-HT2A, 5-HT2B, 5-HT2C, 5-HT6, and 5-HT7. DMT acts as an agonist at 5-HT1A, 5-HT2A and 5-HT2C, with strong binding affinity for the 5-HT2B receptor. DMT also has affinity for the dopamine D1, α1-adrenergic, α2-adrenergic, imidazoline-1, and σ1 receptors. It has also been shown in vitro to be a substrate for the cell-surface serotonin transporter (SERT) expressed in platelets, and the vesicular monoamine transporter 2 (VMAT2). Most of DMT's psychedelic effects can be attributed to a functionally selective activation of the 5-HT2A receptor.

### Structure of DMT

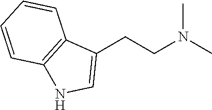

b. Lysergic Acid Diethylamide (LSD)

The dendrimer composition may be conjugated to one or more lysergamides. Amides of lysergic acid are collectively known as lysergamides and include a number of compounds with potent agonist and/or antagonist activity at various serotonin and dopamine receptors. Exemplary lysergamides include but are not limited to d-lysergic acid amide (or d-lysergamide; LSA or LAA), Lysergic acid diethylamide (LSD), ergometrine, DAM-57, ergotamine, Methergine, Methysergide, Amesergide, 2-Bromo-LSD, LSD-Pip, 12-methoxy-LSD, 1P-LSD, 1B-LSD, 1V-LSD, 1 cP-LSD, 13-fluoro-LSD, and 14-hydroxy-LSD. Preferably, the lysergamide conjugated to the dendrimer composition is Lysergic acid diethylamide (LSD) or an LSD derivative.

LSD is an LSD is a semisynthetic ergosterol that can be derived by the naturally occurring ergot alkaloid lysergic acid, which is contained in the rye parasite Claviceps purpurea The mechanism by which LSD works is mainly mediated by activation of serotonin receptors (namely 5HT2A receptors or 5hydroxytryptamine 2A receptor, 5-HT2AR) with modulation of the 5HT2C and 5HT1A receptors. The interactions between the receptor activation and the resulting impairment in cognition and induction of hallucinations are still poorly understood. LSD-induced 5-HT2AR activation leads to a breakdown of inhibitory processes in the hippocampal prefrontal cortex. Specifically, LSD reduces brain activity in the right middle temporal gyrus, superior/middle/inferior frontal gyrus, anterior cingulate cortex, and the left superior frontal and postcentral gyrus and cerebellum. Activation of the right hemisphere, alters thalamic functioning, and increases activity in the paralimbic structures and the frontal cortex; leading to the formation of induced visual imageries.

### Structure of LSD

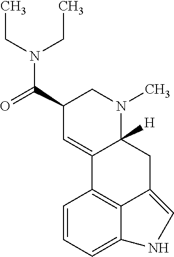 [SMILES: CCN(CC)C(=O)[C@@H]1C=C2c3cccc4[nH]cc(c34)C[C@H]2N(C)C1]

c. Phenethylamines

The dendrimer composition may be conjugated to one or more phenethylamins. Phenethylamine (PEA) is an organic compound, natural monoamine alkaloid, and trace amine, which acts as a central nervous system stimulant in humans. In the brain, phenethylamine regulates monoamine neurotransmission by binding to trace amine-associated receptor 1 (TAAR1) and inhibiting vesicular monoamine transporter 2 (VMAT2) in monoamine neurons. To a lesser extent, it also acts as a neurotransmitter in the human central nervous system. Phenethylamine is produced from the amino acid L-phenylalanine by the enzyme aromatic L-amino acid decarboxylase via enzymatic decarboxylation.

Phenethylamine releases norepinephrine and dopamine, induces acetylcholine release via glutamate mediated mechanisms, and binds to trace amine-associated receptor 1 (TAAR1) as an agonist. Exemplary phenethylamines that can be conjugated to the dendrimer compositions include mescaline and MDMA.

i. Mescaline

Mescaline or mescalin (3,4,5-trimethoxyphenethylamine) is a naturally occurring psychedelic protoalkaloid of the substituted phenethylamine class andis the active component of psychedelic cacti such as peyote (Lophophora williamsii) and wachuma (Echinopsis pachanoi, also known as San Pedro). Mescaline is biosynthesized from tyrosine which, in turn, is derived from phenylalanine by the enzyme phenylalanine hydroxylase.

Similar to the other classic psychedelics, mescaline is a 5HT_{2A/2C} agonist and one of the most selectively serotonergic psychedelics. Mescaline is also binds and modulates the activity of noradrenergic receptors α1 and α2A as well as the TAAR1 receptor. In some forms, the dendrimer composition can be complexed with mescaline in an amount effective to provide between about 300 and about 500 mg. In some forms, the dendrimer composition can be complexed with mescaline in an amount effective to provide a hallucinogenic effect for about 6 hours to about 8 hours.

### Structure of Mescaline

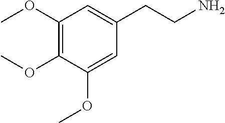

### 2. Entactogens

The dendrimer compositions may include one or more entactogens. Entactogens are Schedule I monoamine releasers and reuptake inhibitors known to evoke a sense of emotional openness and connection such as 3,4-methylenedioxymethamphetamine (MDMA) and 3,4-methylenedioxyamphetamine (MDA). Exemplary entactogens that may be conjugated to the dendrimers include but are not limited to 3,4-methylenedioxymethamphetamine (MDMA), 3,4-methylenedioxy-N-ethyl-amphetamine (MDEA), 3,4-Methylenedioxyamphetamine (MDA), 3,4-Methylenedioxy-N-hydroxyamphetamine (MDOH), 1,3-Benzodioxolyl-N-methylbutanamine (MBDB), 6-APB, methylone, mephedrone, GBL, αMT, MDAI and related compounds.

a. MDMA

The dendrimer composition may be complexed with MDMA. MDMA increases the amount of serotonin in the synaptic clefts of serotonergic neurons by inhibiting its uptake into neurons and by directly releasing it from the neurons. The released serotonin binds to various serotonin receptors and activates them in excess, the primary mechanism through which MDMA causes intoxication. MDMA also induces significant norepinephrine release.

Extracellular MDMA binds to presynaptic serotonin (SERT), norepinephrine (NET) and dopamine transporters (DAT) as a reuptake inhibitor, so that they uptake less of their namesake monoamine neurotransmitters. The efficacy of MDMA inhibition is highest towards NET and SERT, and is much less towards DAT. As a result, more norepinephrine and serotonin remain in the synaptic cleft. These monoamine transporters (SERT, NET and DAT) reuptake their respective neurotransmitters via ion gradient. Normally there is a high extracellular sodium (Na+) and chloride (Cl−) ion concentration, and low potassium (K+) ion concentration in respect to intracellular concentrations. In each reuptake cycle, one monoamine, Na+ and Cl− are simultaneously taken inside the cell. One intracellular K+ is then transported outside the cell. In experimental situations, high extracellular K+ concentration can cause reverse transport. MDMA can substitute for K+ in this transport. Thus, high extracellular MDMA can reverse the transport.

Intracellular MDMA binds to VMAT2-proteins on synaptic vesicles as an inhibitor. Each VMAT2 transports cytosolic monoamines into the vesicle by dissipating proton gradient across the vesicular membrane. Thus, VMAT2 inhibition results in more free cytosolic monoamines like serotonin in the case of serotonergic neurons. These monoamines can then be released via monoamine transporters, which have been reversed by MDMA. Intracellular MDMA can also bind to monoamine oxidase A (MAO-A) as an inhibitor, and thus prevent it from breaking down cytosolic serotonin. MDMA binds as an agonist to the following receptors: 5-HT1A-, 5-HT2A-, 5-HT2B, 5-HT2C-serotonin receptors; α1-, α2A-, β-adrenergic receptors, D1, and D2 dopamine receptor, M1 and M2 muscarinic receptor, H1 histamine receptor, and TAT (TAAR1) receptor.

### Structure of MDMA

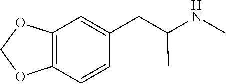

### 3. Dissociative Anesthetics

Another class of drugs with hallucinogenic properties that may be conjugated to the dendrimer compositions are psychedelic or dissociative anesthetics including arylcyclohexamines (also known as arylcyclohexylamines). Arylcyclohexamines are a group of compounds that contain a cyclohexamine unit with an aryl moiety, typically a phenyl ring, attached to the same atom to which the amine group is linked. They all exhibit dissociative effects due to their antagonism of N-methyl-d-aspartate (NMDA) receptors. Exemplary arylcyclohexamines that may be conjugated to the dendrimer compositions include phencyclidine (1-(1-phencyclohexyl) piperidine; PCP), dextromethorphan (DXM), nitrous oxide, ketamine, and methoxetamine (a ketamine analogue).

a. Ketamine

The dendrimer compositions may be conjugated to ketamine (R-ketamine, S-ketamine, (R/S)-ketamine), ketamine analogues, ketamine metabolites, 2-(2-chlorophenyl)-2-(methylamino)-cyclohexanone or analogs thereof, such as KEA-1010, methoxetamine, norketamine, and 2-Fluorodeschloroketamine.

(Ketamine) is a nonbarbiturate dissociative anesthetic. Its beneficial effect is the “dissociation” of brain stem functions from higher brain areas, which alters the sensation of pain and other stimuli during medical procedures, and produces amnesia to the event. It is a cyclohexanone derivative that is rapidly acting and produces profound anesthesia and analgesia. Its chemical name is ±)-2-(o-chlorophenyl)-2-(methylamino) cyclohexanone hydrochloride; its structural formula is CHClNO. Ketamine is a noncompetitive N-methyl-D-aspartate (NMDA) and glutamate receptor antagonist. It blocks HCN1 receptors. The unique dissociative action and partial agonism on opiate mu-receptors permit the performance of painful procedures in a consistent state of sedation and patient comfort.

Ketamine's effects in chronic pain, and as an antidepressant, compare with the actual drug levels and are potentially mediated by a secondary increase in structural synaptic connectivity that is mediated by a neuronal response to the ketamine-induced hyper-glutamatergic state.

Ketamine may interact with the sigma receptors. It tends to work by decreasing central sensitization, wind-up phenomenon (development of ongoing, worsening, or chronic pain), and pain memory. Cholinergic, aminergic, and opioid systems appear to play both a positive and negative modulatory role in both sedation and analgesia. Ketamine reverses tolerance to opioids. It is metabolized via the hepatic system by way of N-dealkylation, hydroxylation, conjugation, and dehydration. The half-life of ketamine is approximately 45 minutes.

In some forms, the recommended doses of ketamine for anesthetic induction are about 1 to 4.5 mg/kg IV and about 6.5 to 13 mg/kg IM, with alternate, off-label recommendations for 0.5 to 2 mg/kg IV and 4 to 10 mg/kg IM, primarily in the context of adjuvant drug use. For use in depression, ketamine is most commonly administered at a sub-anesthetic dose of 0.5 mg/kg IV across 40 minutes.

Conjugation to the dendrimer may further improve ketamine safety and efficacy. For example, dendrimer conjugation may change specific receptor activity and/or modify biodistribution (e.g., use of higher generation dendrimers to confine ketamine to the peripheral nervous system in order to preclude its psychoactive effects).

### Structure of Ketamine

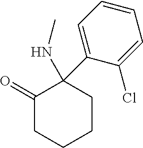 [SMILES: CNC1(c2ccccc2Cl)CCCCC1=O]

b. PCP

Phencyclidine (PCP) (also known as penylcyclohexyl piperidine) is a hallucinogen, specifically, a dissociative anesthetic, that can produce a wide variety of physical and behavioral effects. PCP may cause hallucinations and distorted perceptions of sounds.

PCP's most unusual feature is that doses of 5 to 10 mg orally may induce acute schizophrenia, including agitation, psychosis, audiovisual hallucinations, paranoid delusions, and catatonia. Doses greater than 10 mg usually result in coma. Over 50% of adult patients present with the classic toxidrome of PCP intoxication: violent behavior, nystagmus, tachycardia, hypertension, anesthesia, and analgesia.

PCP primarily acts on the NMDA receptor, an ionotropic glutamate receptor, as an NMDA receptor antagonist. PCP also inhibits nicotinic acetylcholine receptors (nAChRs). In some forms, the dendrimer compositions include an analog of PCP with varying potency at nACh receptors and NMDA receptors. The PCP-induced presynaptic nAChRs and NMDA receptor interactions influence postsynaptic maturation of glutamatergic synapses and consequently impact synaptic development and plasticity in the brain. These effects can lead to inhibition of excitatory glutamate activity in certain brain regions such as the hippocampus and cerebellum.

PCP, like ketamine, also acts as a potent dopamine D2 receptor partial agonist and has affinity for the cloned D2High receptor. This activity may be associated with some of the other more psychotic features of PCP intoxication, which is evidenced by the successful use of D2 receptor antagonists (such as haloperidol) in the treatment of PCP psychosis.

### Structure of PCP

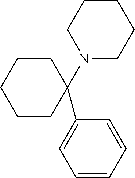 [SMILES: c1ccc(C2(N3CCCCC3)CCCCC2)cc1]

### 4. Ibogaine and Analogs and Derivatives Thereof

Ibogaine is an indole alkaloid isolated from the roots of the West African shrub Tabernanthe iboga. The therapeutic and oneirophrenic (dream-like) effects of iboga roots have been described in the ethnobotanical literature for centuries, where ingestion of Ibogaine root preparations ceremonial and medicinal use.

U.S. Pat. No. 4,499,096 to Lots of, H. S. describes methods for interrupting the narcotic addiction syndrome by administering ibogaine. Oral ibogaine was described as an effective treatment for opioid detoxification. Subsequent studies have shown that Ibogaine decreases drug craving and improves depressive symptoms when administered in a range of 500-1000 mg. This dosage range appears to be a safe and effective treatment for interrupting the opioid addiction syndrome; however, there remain concerns with safety and cardiotoxicity. Similar benefits were observed in recently abstinent cocaine abusers seeking to interrupt their intractable cycle of drug abuse.

Molecular structures of ibogaine and noribogaine illustrate that ibogaine undergoes O)-demethylation to form 12-hydroxyibogamine (noribogaine) by the action of cytochrome P4502D6 (CYP2D6). Ibogaine is metabolized to noribogaine in the gut wall and liver.

18-MC, a synthetic derivative of ibogaine, is an alpha-3-beta-4 nicotinic receptor antagonist with a differentiated mechanism of action that modulates excessive dopamine fluctuations in the mesolimbic system of the brain. 18-MC is a synthetic organic molecule designed around a coronaridine chemical backbone common to a number of plant-based medicinal compounds, including ibogaine. In preclinical efficacy models, 18-MC has demonstrated strong activity in reducing both withdrawal symptoms and self-administration of opioids, stimulants and other substances of abuse. Extensive preclinical characterization has shown 18-MC to have a strong safety and tolerability profile. 18-MC has the potential to overcome safety limitations of ibogaine and has not demonstrated proarrhythmic or neurotoxic activity. Other ibogaine derivatives of include ME-18-MC, 18-MAC, voacangine, ibogamine, and coronaridine.

### 5. Atypical Hallucinogens

In some embodiments, the active agents are one or more atypical hallucinogens having diverse mechanisms such as delta-9-tetrahydrocannabinol or A-9-tetrahydrocannabinol (THC) and ibogaine.

### C. Coupling Agents and Spacers

Dendrimer-active agent conjugates can be formed from one or more active agents covalently conjugated or non-covalently attached to a dendrimer. In preferred embodiments, the one or more active agents are covalently conjugated to the dendrimer.

Optionally, the one or more active agents are conjugated to the dendrimer via one or more spacers. The term “spacer” includes chemical moieties and functional groups used for linking an active agent to the dendrimer. The spacer can be either a single chemical entity or two or more chemical entities linked together. The spacer can include any small chemical entity, peptide or polymers having sulfhydryl, thiopyridine, succinimidyl, maleimide, vinylsulfone, carbonate, etc.

In some embodiments, the spacer via which the active agent is conjugated to the dendrimer contains different linkages such as disulfide, ester, carbonate, carbamate, thioester, hydrazine, hydrazides, ether, and amide linkages. The spacer between a dendrimer and an active agent can be designed to provide a releasable or non-releasable form of the dendrimer conjugate in vivo. In some embodiments, the conjugation between active agent and dendrimer is via an appropriate spacer that contains an ester bond between the active agent and the dendrimer. In some embodiments, one or more spacers between a dendrimer and active agents can provide desired and effective release kinetics in vivo. These spacers may contain cleavable linkages (e.g., ester, disulfide, phosphodiester, triglycyl peptide, and hydrazine) or non-cleavable linkages (e.g., amide, ether, and amino alkyl). The conjugation between active agents and dendrimers can be performed using reaction known in the art, such as click chemistry, acid-amine coupling, Steglich esterification, etc.

In some embodiments, the conjugation between active agent and dendrimer is via a spacer that contain disulfide, ester, ether, thioester, carbamate, carbonate, hydrazine, ether, or amide linkages, or a combination thereof. In some embodiments, the conjugation between active agent and dendrimer is via an appropriate spacer that contain an ester linkage or an amide linkage between the agent and the dendrimer depending on the desired release kinetics of the agent.

The spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido) hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. The spacer can be thiosalicylic acid and its derivatives, (4-succinimidyloxycarbonyl-methyl-alpha-2-pyridylthio) toluene, (3-[2-pyridithio]propionyl hydrazide, The spacer can have maleimide terminations wherein the spacer includes polymer or small chemical entity such as bis-maleimido diethylene glycol and bis-maleimido triethylene glycol, Bis-Maleimidoethane, bismaleimidohexane. The spacer can include vinylsulfone such as 1,6-Hexane-bis-vinylsulfone. The spacer can include thioglycosides such as thioglucose. The spacer can be reduced proteins such as bovine serum albumin and human serum albumin, any thiol terminated compound capable of forming disulfide bonds. The spacer can include polyethylene glycol having maleimide, succinimidyl, and/or thiol terminations.

### D. Dendrimer-Agent Conjugates or Complexes

Dendrimer-active agent conjugates can be formed of antidepressant and/or antipsychotic agents covalently conjugated or non-covalently attached to a dendrimer, a dendritic polymer, or a hyperbranched polymer. Methods for conjugation of one or more active agents to a dendrimer are known, such as those described in U.S. Published 2011/0034422, 2012/0003155, and 2013/0136697. In general, conjugation to the dendrimer may further improve safety and efficacy of these agents. For example, dendrimer conjugation may change specific receptor activity and/or modify biodistribution. For example, use of higher generation dendrimers and/or dendrimers with molecular weights greater than 24 kDa can confine these agents to the peripheral nervous system in order to preclude their psychoactive effects.

In some embodiments, one or more active agents are covalently conjugated to one or more terminal groups of the dendrimer such as terminal hydroxyl groups. In some embodiments, dendrimer conjugates include one or more active agents conjugated to the dendrimer via one or more spacers. The spacer between a dendrimer and an active agent can be designed to provide a releasable or non-releasable form of the dendrimer conjugate in vivo. For example, the spacer can be cleavable or contain a chemical linkage that is cleavable, for example, by exposure to the intracellular compartments of target neural and/or glial cells or upon binding to the receptor on the surface or in the interior of the target neural and/or glial cells in vivo. Examples of cleavable linkages that can be used in a spacer of the dendrimer-active agent conjugates include, esterase sensitive ester bond, glutathione sensitive disulfide bond, phosphatase-sensitive phosphodiester bond, oligopeptide such as triglycyl peptide linker capable of lysosomal release, acid cleavable hydrazine linkage etc. In some embodiments, the spacer between a dendrimer and active agents can provide desired and effective release kinetics in vivo. In some embodiments, the spacer between the dendrimer and the active agent can be non-cleavable or contain a chemical linkage that is non-cleavable, such as amide, ether, and amino alkyl linkages.

Generally, the spacer between the dendrimer and active agent has a length sufficient for the active agent conjugated thereto to reach and bind to the target receptor on the surface and/or inside of the target cell. For example, the spacer between the dendrimer and active agent has a length in a range from 50 Da to 2000 Da, depending on the release kinetics desired, and the receptor binding flexibility desired. The length of the spacer can vary, depending on the location of the target receptor (for example, on the cell surface, in the cytoplasm of the cell, or in an intercellular compartment of the cell) and/or density of the receptor when located on the cell surface.

The dendrimer can be a generation 2, generation 3, generation 4, generation 5, generation 6, and up to generation 10. In some embodiments, the dendrimer is conjugated to one or more active agents via spacers containing cleavable (ester, disulfide, phosphodiester, triglycyl peptide, and hydrazine) or non-cleavable (amide, ether, and amino alkyl) linkages.

The density of active agents covalently conjugated to or non-covalently attached to the dendrimer can be adjusted based on the specific antidepressant and/or antipsychotic agent being delivered, the target receptors, the target neural and/or glial cells, the location of the target neural and/or glial cells, etc. For example, a plurality of active agents conjugated to the dendrimer are on the periphery of the dendrimer and the surface density of the active agent is at least 1 active agent/nm² (number of active agent conjugated/surface area in nm²). For example, in some embodiments, the surface density of active agent per nm² is more than 0.5, 1, 2, 3, 4, 5, 6, 7, 8, 9, 10 OH/nm², such as at least 10, 15, 20, 25, 30, 35, 40, 45, 50, or more than 50 OH/nm². In some embodiments, the volumetric density of active agent is between about 1 and about 50 groups/nm³, between about 5 and about 30 groups/nm³, or between about 10 and about 20 groups/nm³.

Typically, the dendrimer-active agent conjugates have a hydrodynamic volume in the nanometer range. For example, in some embodiments, the glucose dendrimer-active agent conjugates including one or more antidepressant and/or antipsychotic agents conjugated to the dendrimer have a diameter of about 2 nm to about 100 nm, or more than 100 nm, up to 500 nm, depending upon the generation of dendrimer, the chemical composition and amount of active agent conjugated thereto. In some embodiments, a dendrimer-active agent conjugate including one or more antidepressant and/or antipsychotic agents conjugated to the dendrimer has a diameter effective to penetrate brain tissue and to retain on the surface and/or in target neural and/or glial cells for a period of time sufficient for the active agent to bind to the targeted receptors on the surface and/or in the target neural and/or glial cells. In some embodiments, a dendrimer-active agent conjugate including one or more antidepressant and/or antipsychotic agents conjugated to the dendrimer has a diameter effective to remain in the peripheral circulation and to retain on the surface and/or in target neural and/or glial cells for a period of time sufficient for the active agent to bind to the targeted receptors on the surface and/or in the target neural and/or glial cells such as for example, neural and/or glial cells of the gastrointestinal system.

The dendrimer-active agent conjugates can be neural, have a positive charge or a negative charge. In some embodiments, the dendrimer-active agent conjugates are neutral. The presence of antidepressant and/or antipsychotic agents derivatives can affect the surface charge of the dendrimer-active agent conjugates. In some embodiments, the surface charge of the dendrimer conjugated to antidepressant and/or antipsychotic agents is between −100 mV and 100 mV, between −50 mV and 50 mV, between −25 mV and 25 mV, between −20 mV and 20 mV, between −10 mV and 10 mV, between −10 mV and 5 mV, between −5 mV and 5 mV, or between −2 mV and 2 mV. The range above is inclusive of all values from −100 mV to 100 mV. In preferred embodiments, the surface charge of the dendrimer-active agent conjugates is neutral or near-neutral, i.e., from about −10 mV to about mV, inclusive.

An exemplary dendrimer-active agent conjugate is represented by Formula (I). The dendrimer of the exemplary conjugate contains surface hydroxyl groups, wherein one or more of the surface hydroxyl groups are conjugated to one or more active agents via one or more spacers as shown in Formula (I) below.

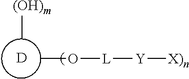

Wherein D can be a generation 1 to generation 10 or generation 2 to generation 10 dendrimer, such as any one of those described above, for example, PAMAM (such as hydroxyl-terminated PAMAM dendrimer) or a glucose-based dendrimer; each occurrence of L can be any suitable chemical moiety, preferably containing a triazole moiety; Y can be a bond or a linkage selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, phosphodiester group

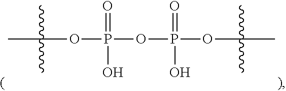 [SMILES: *OP(=O)(O)OP(=O)(O)O*]

hydrazino group, hydrazones, hydrazides, ester (—C(O)—O—), ether (—O—), and oligopeptide (e.g., triglycyl peptide), wherein R is an alkyl group, an aryl group, or a heterocyclic group; each occurrence of X can be a antidepressant and/or antipsychotic agent, wherein a functional group of X (such as an amino group including primary amino, secondary amino, or tertiary amino group; a carboxylic group; or a hydroxyl group) forms a portion of linkage Y; n can be an integer from 1 to 100; and m can be an integer from 16 to 4096. The dendrimer can be PAMAM (such as hydroxyl-terminated PAMAM) or a glucose dendrimer, which is 100% hydroxyl. m and n depend on the size of the dendrimer D, n should be such that the weight percent of the drug in the total conjugate is 5-20%. This range is also appropriate for binding and internalization.

The oxygen atom shown in Formula (I) is from the surface functional group of the dendrimer, such as a surface hydroxyl group, where the surface hydroxyl group may or may not be part of a terminal sugar moiety/molecule (e.g., glucose). Although not illustrated in Formula (I), one or more hydroxyl groups of the dendrimer that are not conjugated to active agents may be modified with one or more carbohydrates and/or polyalkylene glycols, such as PEG.

When administered to a subject in need thereof, the antidepressant and/or antipsychotic agent X of Formula (I) can bind to a target receptor on the surface of the target cell or inside the target cell. In some embodiments, when the antidepressant and/or antipsychotic agent X binds to the target receptor, the agent X remains conjugated to the dendrimer. In these embodiments, following binding, the agent X may be released from the dendrimer or remain conjugated to the dendrimer as an intact dendrimer-active agent conjugate. In some embodiments, the antidepressant and/or antipsychotic agent X is released from the dendrimer at close proximity to the target receptor and then binds to the target receptor.

In some embodiments, each occurrence of L can be represented by -A′-L1-B′-L2-, wherein A′ can be a carbonyl (—C(O)—) or a bond (including single, double, and triple bonds, for example a single bond); B′ can be a bond (including single, double, and triple bonds, for example a single bond), an amide, an ester, an ether, a thiol, a dithiol, an aryl, a heteroaryl, a polyaryl, a heteropolyaryl, or a heterocyclic; and L1 and L2 can be independently a bond, an alkylene, a heteroalkylene, an aryl, an aralkyl, an ether, a polyether, a thiol, a dithiol, a thiolether, a polythioether, an oligopeptide, a polypeptide, an oligo (alkylene glycol), or a polyalkylene glycol, or L1 and L2 can be independently composed of a combination of these groups, such as a combination of alkylene and polyether, a combination of alkylene and thiol or dithiol, a combination of alkylene and oligopeptide, a combination of alkylene, polyether, and thiol or dithiol, or a combination of polyether and thiol or dithiol. In some forms, L1-B′-L2-together form a chemical moiety selected from an -alkylene-triazole-di(alkylene glycol)-, a -di(alkylene glycol)-triazole-alkylene-, -alkylene-triazole-oligo (alkylene glycol)-, an -oligo (alkylene glycol)-triazole-alkylene-, an -alkylene-triazole-poly(alkylene glycol)-, -poly(alkylene glycol)-triazole-alkylene-, an -alkylene-triazole-ether-, an -alkylene-triazole-alkylene-, an -alkylene-amide-alkylene-, and combinations thereof.

In some embodiments, B′ can be a bond (including single, double, and triple bonds, for example a single bond), an amide group, or a heterocyclic group, such as a triazole group.

In some embodiments, L1 can be a bond; an alkylene, such as a C₁-C₁₀ alkylene, a C₁-C₈ alkylene, a C₁-C₆ alkylene, a C₁-C₅ alkylene, a C₁-C₄ alkylene, or a C₁-C₃ alkylene; or an oligo- or poly-(alkylene glycol), such as

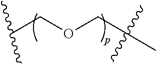

where p is an integer from 1 to 20, from 1 to 18, from 1 to 16, from 1 to 14, from 1 to 12, from 1 to 10, from 1 to 8, from 1 to 6, from 1 to 5, from 1 to 4, from 1 to 3, or 1 or 2.

In some embodiments, L2 can be a bond; an alkylene, such as a C₁-C₁₀ alkylene, a C₁-C₈ alkylene, a C₁-C₆ alkylene, a C₁-C₅ alkylene, a C₁-C₄ alkylene, or a C₁-C₃ alkylene; an oligo- or poly-(alkylene glycol), such as

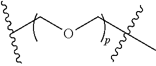

where p is an integer from 1 to 20, from 1 to 18, from 1 to 16, from 1 to 14, from 1 to 12, from 1 to 10, from 1 to 8, from 1 to 6, from 1 to 5, from 1 to 4, from 1 to 3, or 1 or 2; an oligo- or poly-peptide, such as a triglycyl peptide; a thiol; or a dithiol; or L2 is composed of a combination of two or more of alkylene, oligo- or poly-(alkylene glycol), oligo- or poly-peptide, thiols, and dithiols. For example, L2 is represented by

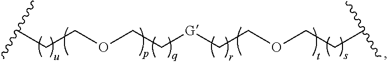

where p, q, r, s, t, and u are independently an integer from 0 to 10, from 0 to 8, from 0 to 6, from 0 to 5, from 0 to 4, from 0 to 3, or from 0 to 2, such as 0, 1, or 2; and G′ is a thiol, a dithiol, an oligo-peptide such as a triglycyl peptide, or a poly-peptide.

In some embodiments, Y is a linkage that is minimally cleavable in vivo. In some embodiments, Y is a linkage that is cleavable in vivo. In some embodiments, Y is an amide (—CONH—), an ester (—C(O)—O—), an ether (—O—), a phosphodiester, or a disulfide group.

In some embodiments, L and Y are both a single bond, and D is directly conjugated to X (an active agent or analog thereof) via an ether linkage.

In some embodiments, D is a generation 2 PAMAM dendrimer, a generation 3 PAMAM dendrimer, a generation 4 PAMAM dendrimer, a generation 5 PAMAM dendrimer, a generation 6 PAMAM dendrimer, a generation 1 glucose dendrimer, a generation 2 glucose dendrimer, a generation 3 glucose dendrimer, a generation 4 glucose dendrimer, a generation 5 glucose dendrimer, or a generation 6 glucose dendrimer.

More specific exemplary dendrimer-active agent conjugates are shown in the Examples below.

### E. Exemplary Dendrimer-Agent Conjugates

In preferred embodiments, the dendrimer is conjugated to psilocin, or a psilocin analog as shown in Structures A-D below.

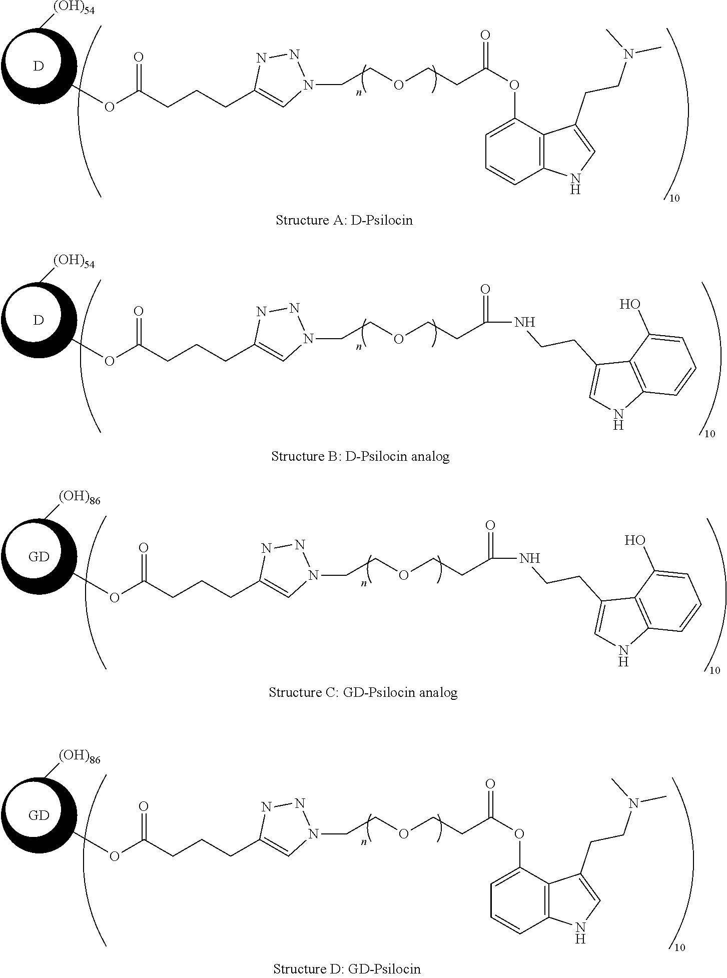

n is an integer from 1 to 10, such as 1, 2, 3, 4, 5, 6, 7, 8, 9, or 10.

In preferred embodiments, the dendrimer is conjugated to ketamine, or a ketamine analog as shown in Structures E and F below.

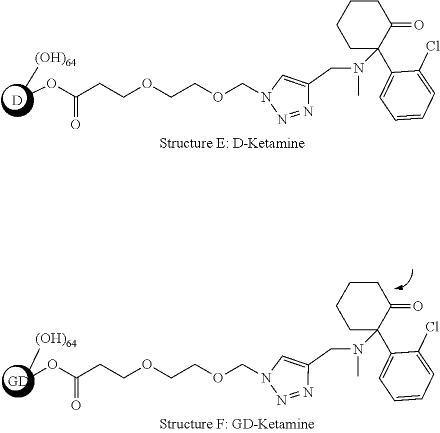

In other preferred embodiments, the dendrimer is conjugated to DMT, or a DMT analog as shown in Structures G-J below.

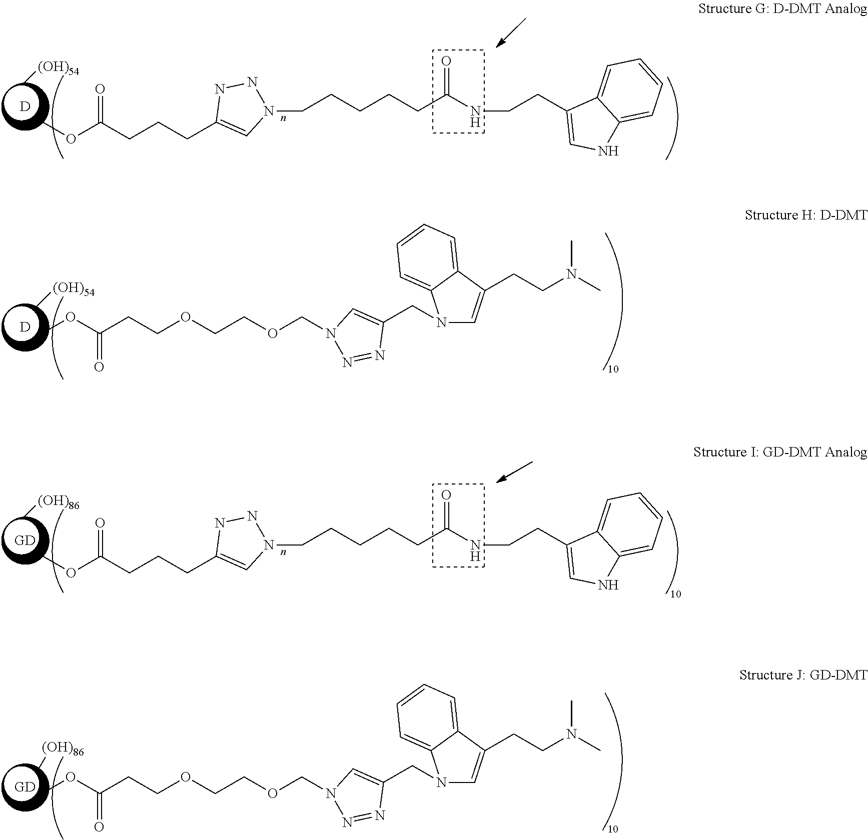

In preferred embodiments, the dendrimer is conjugated to LSD, or a LSD analog as shown in Structures K and L below.

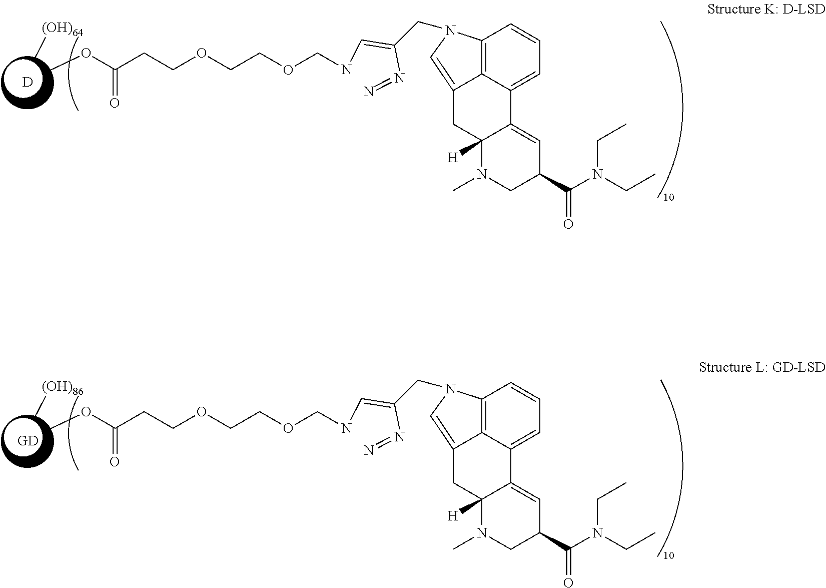

### III. Methods of Making Dendrimer Conjugates

Methods of synthesizing dendrimers and making dendrimer nanoparticles are also described.

### A. Methods of Making Dendrimers

Dendrimers can be prepared via a variety of chemical reaction steps.

Dendrimers are usually synthesized according to methods allowing controlling their structure at every stage of construction. The dendritic structures are mostly synthesized by two main different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward, building inward, and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more spacers, and/or one or more surface functional groups can be modified to allow conjugation to further functional groups (branching units, spacers, surface functional groups, etc.), monomers, and/or agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5): 9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface functional group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc. on the second moiety. In some embodiments, one or more hydroxyl groups on the surface of the dendrimer (hydroxyl-terminated PAMAM dendrimer or glucose dendrimer) are modified to contain an alkyl group and a drug is modified to contain an azide group. Alternatively, one or more hydroxyl groups on the surface of the dendrimer (hydroxyl-terminated PAMAM dendrimer or glucose dendrimer) are modified to contain an azide group and a drug is modified to contain an alkyne group. The azide and alkyne are then reacted via a 1,3-dipolor cycloaddition reaction to form a triazole moiety.

In some embodiments, dendrimer synthesis relies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

In some embodiments, methods involve one or more protection and deprotection steps of the function groups (e.g., hydroxyl groups) on the central core, branching units, and/or therapeutic, prophylactic or diagnostic agents to facilitate addition of branching units to generate desired dendrimer molecules, or addition of therapeutic, prophylactic or diagnostic agents to generate desired dendrimer conjugates. In the case of hydroxyl groups, they may be protected by formation of an ether, an ester, or an acetal. Other exemplary protection groups include Boc and Fmoc.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly(propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of agents is linked to one type of dendron and a different type of agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in International Patent Publication Nos. WO2009/046446, WO2015168347, WO2016025745, WO2016025741, WO2019094952, and U.S. Pat. No. 8,889,101.

### 1. Methods of Making Glucose Dendrimers

The glucose-based dendrimers are assembled from a multifunctional core, which is extended outward by a series of reactions. The strategy involves the coupling of monomeric molecules that possess reactive and protective groups with the multifunctional core moiety, which leads to stepwise addition of generations around the core followed by removal of protecting groups.

In some embodiments, glucose dendrimers are synthesized by coupling AB₄ peracetylated β-D glucose-PEG4-azide monomers to hexapropargylated core. In preferred embodiments, the hypercore is prepared from dipentaerythritol, for example by performing propargylation of dipentaerythritol to achieve the hexa-propargylated core. An exemplary scheme for preparing such a glucose dendrimer is shown by Scheme I.

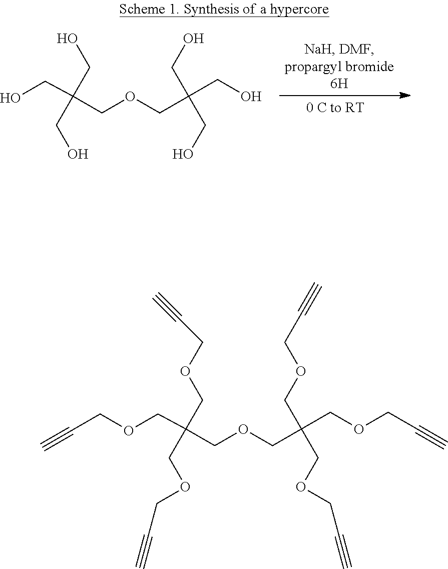 [SMILES: OCC(CO)(CO)COCC(CO)(CO)CO ; C#CCOCC(COCC#C)(COCC#C)COCC(COCC#C)(COCC#C)COCC#C]

In some embodiments, the branching units are hypermonomers i.e., ABₙ building blocks. Exemplary hypermonomers include AB₃, AB₄, AB₅, AB₆, AB₇, AB₈ building blocks. Hypermonomer strategy drastically increases the number of available end groups. An exemplary hypermonomer is AB₄ orthogonal hypermonomer including one azide functional group and four allyl groups prepared from dipentaerythritol with five allyl groups reacted with mono tosylated triethylene glycol azide.

In some embodiments, the branching unit is polyethylene glycerol linear or branched e.g., as shown by Formula III. Other monomers include disaccharides and oligosaccharides, as well as saccharides such as fructose, lactose, and sucrose.

a. Synthesis of AB4 Building Block

Some exemplary synthesis methods of hypermonomer AB4 are described below. In some embodiments, the hypermonomer AB4 is based on glucose molecules. In preferred embodiments, the hypermonomer AB4 is conjugated to a polyethylene glycerol, for example, tetraethylene glycol (PEG4). In one embodiment, the hypermonomer AB4 is peracetylated β-D-Glucopyranoside tetraethylene glycol azide.

In some embodiments, the synthesis of glucose-Oac-TEG-Ots involves the following steps: a solution of peracetylated β-D-glucopyranoside (10 g, 25.6 mmol) was dissolved in 50 mL of anhydrous dichloromethane (DCM) followed by addition of 2-(2-(2-(2-hydroxyethoxy) ethoxy) ethoxy)ethyl 4-methylbenzenesulfonate (6.2 g, 17.9 mmol) and the reaction mixture was cooled to 0° C. Boron trifluoride diethyl etherate (2.5 eq.) was added and the reaction was allowed to come to room temperature. The reaction was monitored with the help of TLC and quenched after 5 hrs by the addition of saturated sodium bicarbonate solution at 0° C. After 10 minutes of stirring, DCM (300 mL) was added and the organic layer was washed with saturated sodium bicarbonate solution 3 times until the effervescence was quenched. The reaction mixture was dried over sodium sulfate, filtered, and evaporated under reduced pressure. The crude product was purified by combiflash chromatography using ethyl acetate/hexanes (70:30) mixture as eluents. The desired compound was achieved in 60% yield. Structure of glucose-Oac-TEG-Ots is shown below:

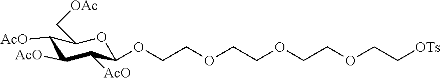

In some embodiments, the synthesis of glucose-Oac-TEG-N₃ involves the following steps: a solution of glucose-Oac-TEG-Ots (6 g, 8.8 mmoles) is dissolved in 40 mL of anhydrous DMF followed by the addition of sodium azide (2 eq) and the reaction mixture is heated to 50° C. for overnight. Upon completion, the reaction mixture is filtered and DMF is evaporated. Once dried, the crude reaction mixture is passed through combiflash using ethyl acetate:hexane (70:30) as eluent. Structure of glucose-Oac-TEG-N₃ is shown below:

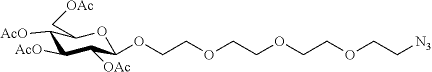

In some embodiments, the synthesis of glucose-OH-TEG-N₃ involves the following steps: the peracetylated β-D-Glucopyranoside tetraethylene glycol azide is dissolved in anhydrous methanol and sodium methoxide is added to adjust the pH around 8.5-9. The reaction is stirred overnight at room temperature, then diluted with methanol and pH is adjusted with Amberlist IR-120+ around 6-7. The reaction mixture is separated by filtration and the solvent removed by rotary evaporation. Structure of glucose-OH-TEG-N₃ is shown below.

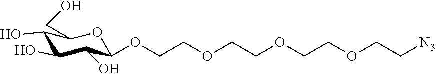

b. Synthesis of Glucose Dendrimers

In some embodiments, glucose dendrimers are synthesized by coupling AB4 peracetylated B-D glucose-PEG4-azide monomers to hexapropargylated core. In preferred embodiments, the hexapropargylated core is linked to AB₄ β-D-glucose-PEG4-azide building block (2) via click reaction to obtain generation 1 dendrimer.

In some embodiments, generation one dendrimer D1-Glu6-Oac24 is prepared according to the following: Hexapropargylated compound (0.5 g, 1 mmoles) and an azido derivative ((4.1 g, 7.4 mmoles) 1.2 eq. per acetylene) are suspended in a 1:1 mixture of DMF and water in a 20 mL microwave vial equipped with a magnetic stir bar. CuSO₄·5H₂O (5 mol %/acetylene, 75 mg) and sodium ascorbate (5 mol %/acetylene, 60 mg) dissolved in the minimum amount of water are added. The reaction is irradiated in a microwave at 50° C. for 6 h. The reaction mixture is dialyzed against DMF followed by water dialysis containing EDTA. The EDTA is further removed by extensive water dialysis. The product is lyophilized to obtain D1-Glu6-Oac24. Structure of D1-Glu6-Oac24 is shown below.

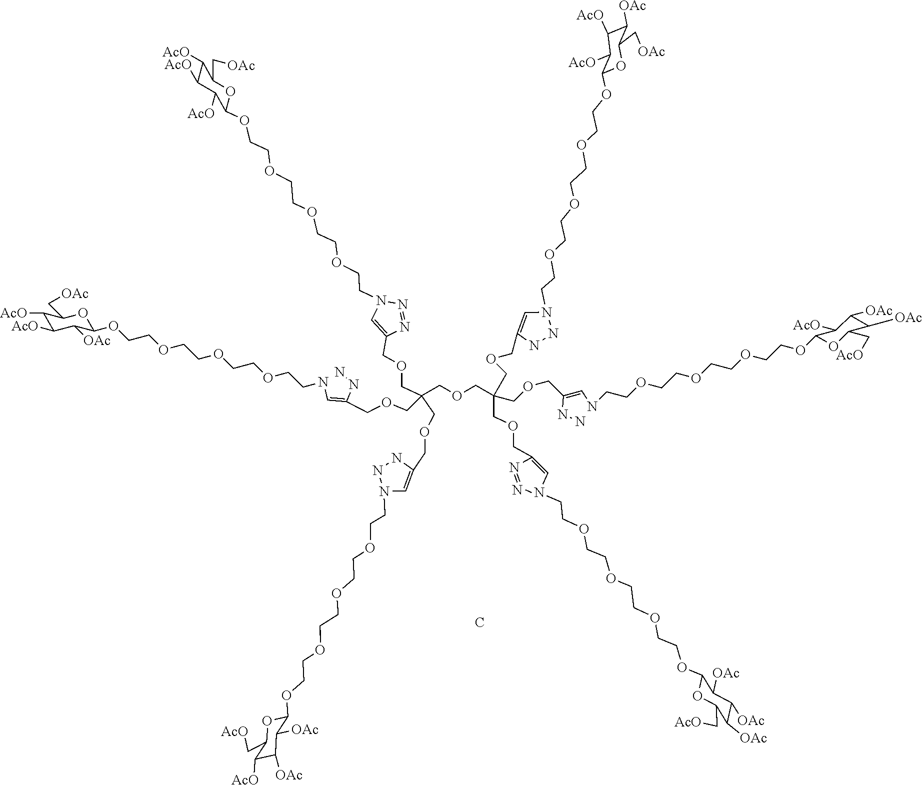

In some embodiments, generation one dendrimer D1-Glu₆-OH₂₄ is prepared according to the following: the peracetylated generation 1 glucose dendrimer (1 g, 0.26 mmoles) is dissolved in anhydrous methanol and sodium methoxide is added to adjust the pH to around 8.5-9. The reaction is stirred overnight at room temperature, then diluted with methanol and pH is adjusted with AMBERLIST® IR-120+ around 6-7. The reaction mixture is separated by filtration and the solvent removed by rotary evaporation, followed by water dialysis. Structure of generation one glucose dendrimer, D1-Glu₆-OH₂₄, is shown below.

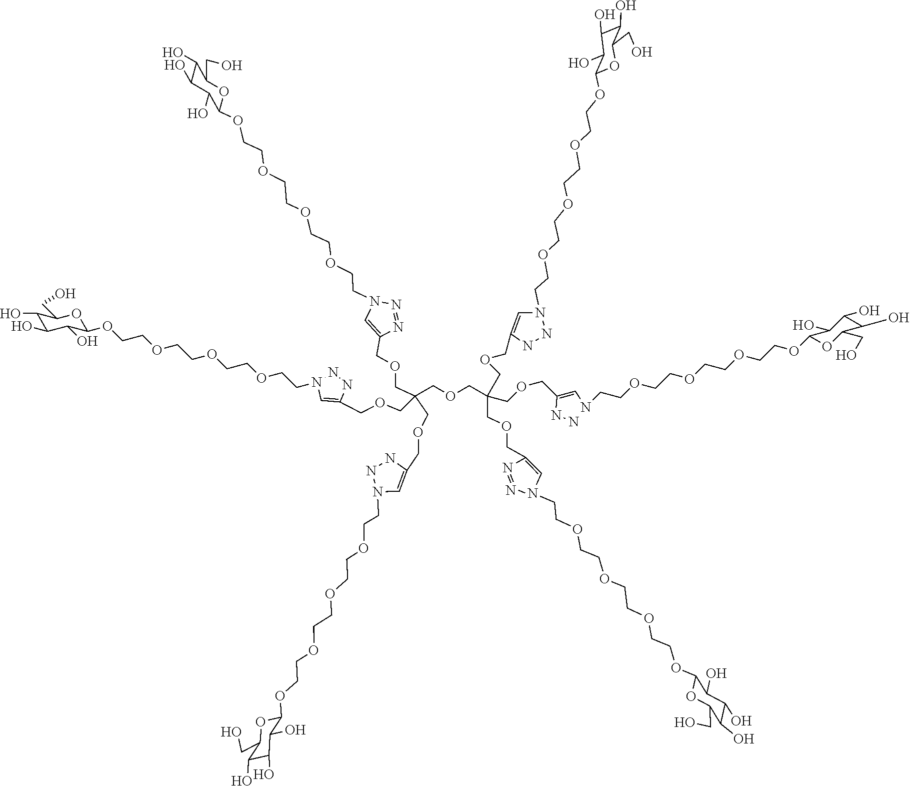

In some embodiments, generation one glucose dendrimer D1-Glu₆-OH₂₄ is propargylated to provide D1-Acetylene24 according to the following: D1-Glu₆-OH₂₄ (2 g, 0.721 mmol) was dissolved in anhydrous dimethylformamide (DMF, 50 mL) by sonication. Sodium hydride [60% dispersion in mineral oil] (951 mg, 39.65 mmol) is slowly added in portions at 0° C. to the solution with stirring. The solution is stirred for an addition 15 minutes at 0° C. This is followed by the addition of propargyl bromide (3.85 mL, 34.608 mmol, 80% w/w solution in toluene) at 0° C. and the stirring is continued at room temperature for another 6 h. The reaction mixture is quenched with ice and water, filtered, and dialyzed against DMF, followed by the water dialysis to afford D1-acetylene24. Structure of D1-acetylene24 is shown below.

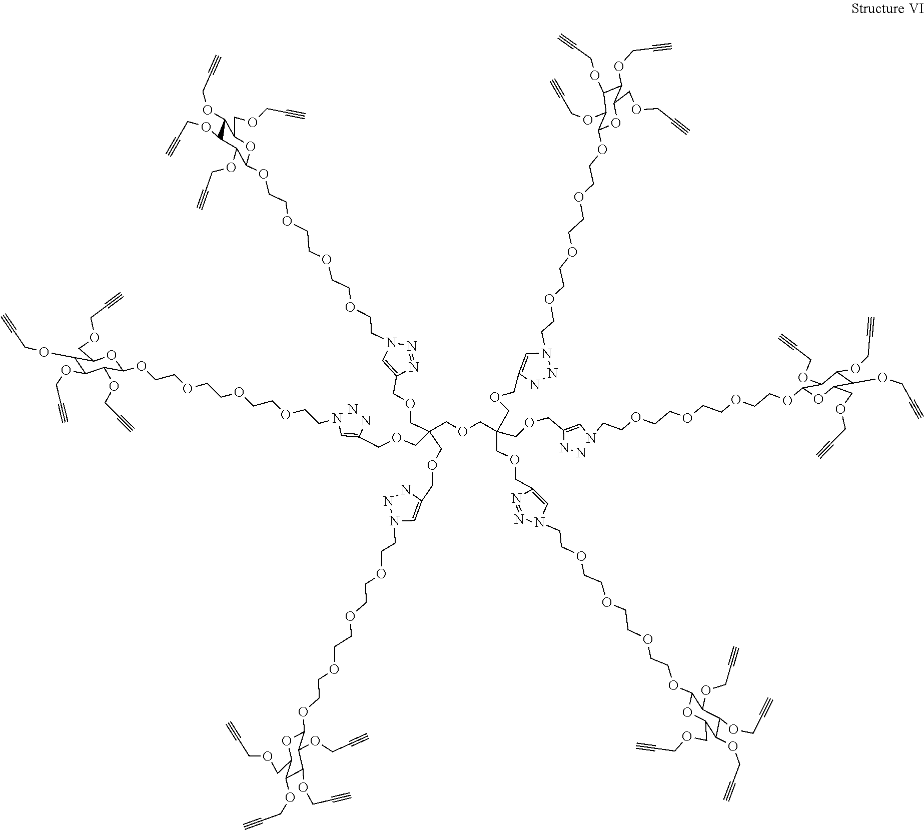

In some embodiments, generation one dendrimer D1-acetylene24 is further reacted with AB₄ β-D-glucose-PEG4-azide to provide generation 2 dendrimer with 24 glucose molecules containing 96 surface hydroxyl groups.

An exemplary generation two dendrimer D2-Glu24-Oac96 is prepared according to the following: D1-acetylene dendrimer24 (0.5 g, 0.13 mmoles) and glucose-Oac-TEG-azide (2.2 g, 4 mmoles) are suspended in a 1:1 mixture of DMF and water in a 20 mL microwave vial equipped with a magnetic stir bar. To this CuSO₄·5H₂O (5 mol %/acetylene, 5 mg) and sodium ascorbate (5 mol %/acetylene, 10 mg) dissolved in the minimum amount of water are added. The reaction is irradiated in a microwave at 50° C. for 8 h. Upon completion, the reaction mixture is dialyzed against DMF followed by water dialysis containing EDTA. The EDTA is further removed by extensive water dialysis. The product is lyophilized to obtain D2-Glu24-Oac96.

In some embodiments, generation two dendrimer D2-Glu₂₄-OH₉₆ is prepared according to the following: the peracetylated generation 2 glucose dendrimer D2-Glu₂₄-OH₉₆ is dissolved in anhydrous methanol and sodium methoxide is added to adjust the pH around 8.5-9.0. The reaction is stirred overnight at room temperature, then diluted with methanol and pH is adjusted with AMBERLIST® IR-120+ around 6-7. The reaction mixture is filtered to remove the resin and the filtrate is evaporated by rotary evaporation followed by water dialysis to obtain the product as off-white solid.

Structure of generation two glucose dendrimer, D2-Glu₂₄-OH₉₆, is shown below.

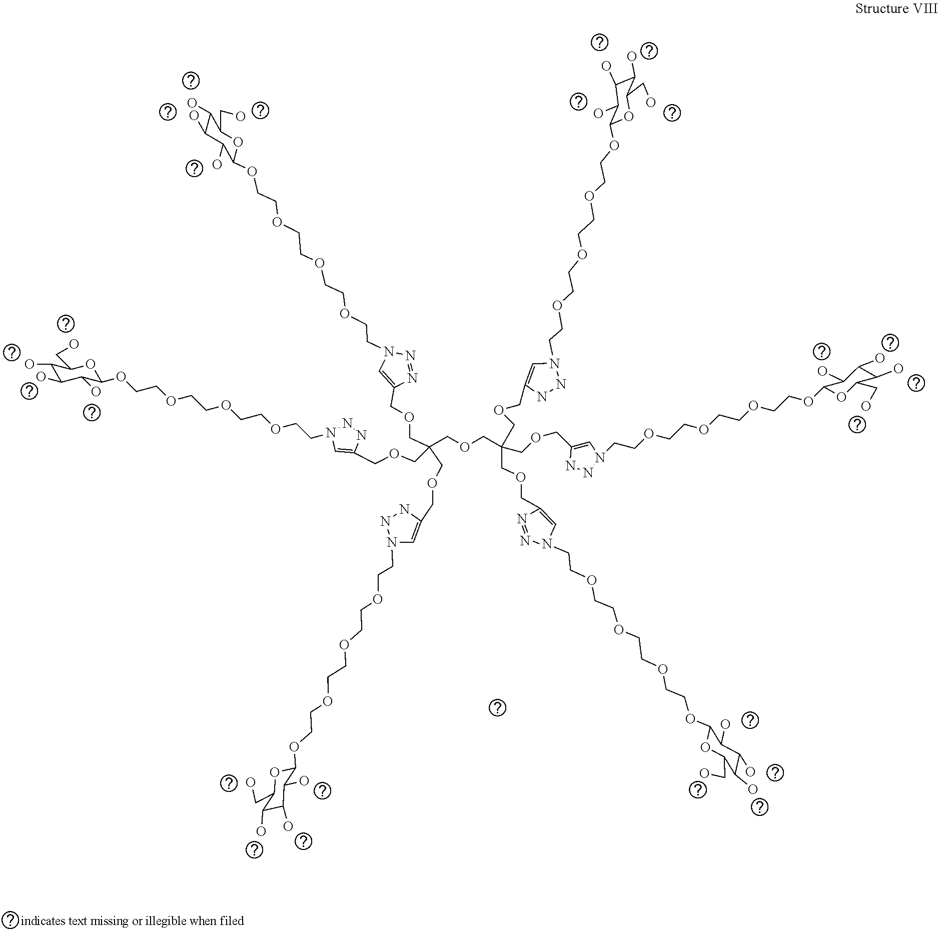

In some embodiments, generation two dendrimer D2-Glu₂₄-OH₉₆ is propargylated at one or more terminal hydroxyl groups suitable for further conjugation to one or more therapeutic, prophylactic or diagnostic agents. In some embodiments, one or more terminal hydroxyl groups of generation two dendrimer D2-Glu24-OH96 is propargylated according to the following: D2-Glu24-OH96 (5b) (200 mg, 0.016 mmol) is dissolved in anhydrous dimethylformamide (DMF, 10 mL) by sonication. To this stirring solution, sodium hydride [60% dispersion in mineral oil] (22 mg, 0.934 mmol) is slowly added in portions at 0° C. The solution is additionally stirred for 15 minutes at 0° C. This is followed by the addition of propargyl bromide (18.0 μL, 80% w/w solution in toluene) at 0° C. and the stirring is continued at room temperature for another 6 h. The solvent is evaporated using V10 evaporator system and the crude product is purified by passing through PD10 SEPHADEX® G25 M column. The aqueous solution is lyophilized to afford the product as off-white solid.

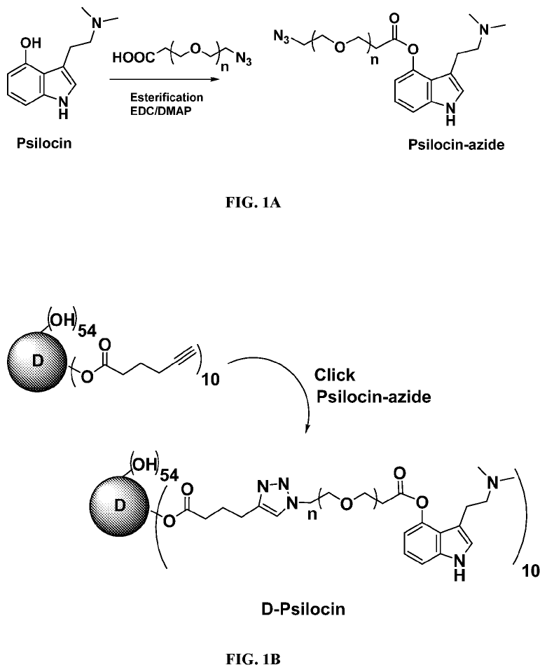

In some embodiments, one or more fluorescent dyes such as infrared fluorescent Cy5 dyes are conjugated to generation two dendrimer D2-Glu24-OH96. In one embodiment, Cy5-D2-Glu24-OH96 (compound 7 of FIG. 1B) is prepared according to the following: Compound 6 (200 mg, 0.016 mmol) and Cy5 azide (20.7 mg, 0.02 mmol) are suspended in a 1:1 mixture of DMF and water in a 25 mL round bottom flask equipped with a magnetic stir bar. To this, CuSO₄·5H₂O (5 mol %/acetylene, 0.3 mg) and sodium ascorbate (10 mol %/acetylene, 0.5 mg) dissolved in the minimum amount of water are added. The reaction is stirred at room temperature for 24 h. Upon completion, the DMF is evaporated using V10 and the purification is performed using PD10 Sephadex G25 M column. The aqueous solution is lyophilized to afford the product as blue solid.

In some embodiments, the total hydroxyl groups for further conjugation to active agents including therapeutic and/or diagnostic agents are about 1-30, 2-20, or 5-10 out of total 96 available hydroxyl groups of the exemplary generation 2 dendrimer with 24 glucose molecules containing 96 surface hydroxyl groups.

### B. Methods of Making Dendrimer-Agent Conjugates

Methods for conjugating agents with dendrimers are generally known in the art, for example, as described in US 2011/0034422, US 2012/0003155, and US 2013/0136697.

In some embodiments, one or more agents are covalently attached to the dendrimers. In some embodiments, the agents are attached to the dendrimer via a spacer that is designed to be non-cleavable in vivo. In some embodiments, the agents are attached to the dendrimer via a spacer that is designed to be cleaved in vivo. For example, the spacer can be designed to be cleaved hydrolytically, enzymatically, or combinations thereof, so as to provide for the sustained release of the agents in vivo. In some embodiments, both the chemical structure of the spacer and its point of attachment to the agent, can be selected so that cleavage of the spacer releases either an agent, or a suitable prodrug thereof. The chemical structure of the spacer can also be selected in view of the desired release rate of the agents.

In some embodiments, the conjugation between the agent and dendrimer is via one or more of disulfide, ester, ether, phosphodiester, triglycyl peptide, hydrazine, amide, or amino alkyl linkages. In some embodiments, the conjugation between the agent and dendrimer is via an appropriate spacer that provides an ester bond or an amide bond between the agent and the dendrimer depending on the desired release kinetics of the agent. In some cases, an ester or disulfide bond is introduced for releasable form of agents. In other cases, an amide or amino alkyl bond is introduced for non-releasable form of agents.

Spacers generally contain one or more organic functional groups. Examples of suitable organic functional groups contained in the spacers include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the spacer is chosen in view of the desired release rate of the agents. In addition, the one or more organic functional groups can be selected to facilitate the covalent conjugation of the agents to the dendrimers. In some embodiments, the conjugation between the agent and dendrimer is via an appropriate spacer that provides a disulfide bridge between the agent and the dendrimer. In some embodiments, the dendrimer-active agent conjugates are capable of rapid release of the agent in vivo by thiol exchange reactions, under the reduced conditions found in body.

In certain embodiments, the spacer contains one or more of the organic functional groups described above in combination with a linking group. The linking group can be composed of any assembly of atoms, including oligomeric and polymeric chains; for example, the total number of atoms in the linking group is between 3 and 200 atoms, between 3 and 150 atoms, between 3 and 100 atoms, or between 3 and 50 atoms. Examples of suitable linking groups include alkyl groups, heteroalkyl groups, alkylaryl groups, oligo- and polyethylene glycol chains, and oligo- and poly(amino acid) chains. Variation of the linking group provides additional control over the release of the agents in vivo. In embodiments where the spacer includes a linking group, one or more organic functional groups will generally be used to connect the linking group to both the anti-inflammatory agent and the dendrimers.

Reactions and strategies useful for the covalent conjugation of agents to dendrimers are known in the art. See, for example, March, “Advanced Organic Chemistry,” 5th Edition, 2001, Wiley-Interscience Publication, New York and Hermanson, “Bioconjugate Techniques,” 1996, Elsevier Academic Press, U.S.A. Appropriate methods for the covalent conjugation of a given agent can be selected in view of the linking moiety desired, as well as the structure of the agents and dendrimers as it relates to compatibility of functional groups, protecting group strategies, and the presence of labile bonds.

The amount of active agent in the dendrimer-active agent conjugates (drug loading) depends on many factors, including the choice of active agent, dendrimer structure and size, and tissues to be treated. In some embodiments, the one or more antidepressant and/or antipsychotic agents are conjugated to the dendrimer at a concentration between about 0.01% and about 45%, inclusive; between about 0.1% and about 30%, inclusive; between about 0.1% and about 20%, inclusive; between about 0.1% and about 10%, inclusive; between about 1% and about 10%, inclusive; between about 1% and about 5%, inclusive; between about 3% and about 20% by weight, inclusive; or between about 3% and about 10% by weight, inclusive. However, specific drug loading for any given active agent, dendrimer, and site of target can be identified by routine methods, such as those described.

In some embodiments, the conjugation of agents/spacers occurs via about 1%, 2%, 3%, 4%, or 5% of the total available surface functional groups, such as hydroxyl groups, of the dendrimers prior to the conjugation. In other embodiments, the conjugation of agents/spacers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation with active agents. In some embodiments, dendrimer-active agent conjugates retain an effective amount of surface functional groups for targeting to target neural and/or glial cells, whilst conjugated to an effective amount of agents for treat, prevent, and/or image the disease or disorder. In some embodiments, dendrimer-active agent conjugates retain an effective amount of active agents for targeting to target neural and/or glial cells and binding to target receptors on the surface or in the interior of the target neural and/or glial cells.

More specific methods for preparing exemplary dendrimer-active agent conjugates are described in the Examples below.

### IV. Pharmaceutical Formulations

Pharmaceutical compositions including dendrimer-active agent conjugates may be formulated in a conventional manner using one or more physiologically acceptable carriers, optionally including excipients and auxiliaries which facilitate processing of the active compounds into preparations which can be used pharmaceutically, for oral, mucosal (intranasal, buccal, sublingual, vaginal, rectal or pulmonary), transdermal, or injection (intravenous, subcutaneous, intraperitoneal, intramuscular, or intrathecal administration).

Representative excipients include aqueous buffers, wetting agents, viscosity modifiers, tonicity agents, stabilizing agents, and combinations thereof. Suitable pharmaceutically acceptable excipients are preferably selected from materials which are generally recognized as safe (GRAS) and may be administered to an individual without causing undesirable biological side effects or unwanted interactions.

Generally, pharmaceutically acceptable salts of the actives can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs, or extrapolated from human data. The animal model is also used to achieve a desirable concentration range and route of administration. Such information should then be useful to determine effective doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and is expressed as the ratio, LD50/ED50. Pharmaceutical compositions which exhibit large therapeutic indices are preferred. Data obtained from cell culture assays and animal studies can be used in formulating a range of dosages for human use.

In certain embodiments, the compositions are administered locally, for example, by injection directly into a site to be treated or by an implant. In some embodiments, the compositions are injected, topically applied, or otherwise administered directly into the vasculature onto vascular tissue at or adjacent to a site of injury, surgery, or implantation. For example, in embodiments, the compositions are topically applied to vascular tissue that is exposed, during a surgical procedure, or as a cream, gel or lotion. Typically, local administration causes an increased localized concentration of the compositions, which is greater than that which can be achieved by systemic administration.

Pharmaceutical compositions formulated for administration by parenteral (intramuscular, intraperitoneal, intravenous, or subcutaneous injection) and enteral routes of administration are described.

### A. Parenteral Administration

The compositions of dendrimer-active agent conjugates can be administered parenterally. The phrases “parenteral administration” and “administered parenterally” are art-recognized terms and include modes of administration other than enteral and topical administration. The dendrimers can be administered orally, intranasally, subcutaneously, intraperitoneally, transdermally, or intramuscularly. For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycols are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

The compositions of dendrimer-active agent conjugates can be administered enterally (orally, sublingually, vaginally, rectally, buccally, intranasally, pulmonarily, or transdermally). The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions, or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides, and other components of infant formulas.

Oral formulations may be in the form of chewing gum, gel strips, tablets, capsules, or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

Formulations for administration to mucosal surfaces such as the nose, buccal surfaces or pulmonary, typically contain pharmaceutically acceptable excipients such as those used for parenteral administration, alone or in combination with various surfactants, penetration enhancers, etc.

The compositions can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

### V. Methods of Use

Methods of using the composition of dendrimer-psychedelic drugs are described. The methods can treat or alleviate one or more symptoms of psychological, cognitive, behavioral, and/or mood disorders. The compositions are administered in an amount effective to provide a therapeutic effect, but insufficient to provide an adverse effect.

In preferred embodiments, the dendrimer compositions traverse the barrier interfaces of the central and peripheral nervous system, and selectively target specific cells and specific receptors on the cells to address a variety of diseases, disorders, injuries, and conditions. The methods include administering to a subject in need, the compositions in an amount effective to increase permeability of the hallucinogenic compound across the barrier interfaces of the central and peripheral nervous system, and/or increase binding of the hallucinogenic compound at specific receptors in specific cells, particularly the serotonergic receptors, dopaminergic receptors, adrenergic, and/or monoamine transporters in the central nervous system, peripheral nervous system, and/or cells in peripheral circulation, e.g., neural cells, glial cells, gastrointestinal cells, and/or immune cells.

### A. Methods of Treatment

The compositions can be administered to prevent, treat, and/or manage the symptoms a variety of disorders, diseases, and conditions including but not limited to anxiety disorders, mood disorders, eating disorders, personality disorders, stress disorders, and/or psychotic disorders. In some forms, when the dendrimer is complexed to one or more hallucinogens, the compositions can be administered to treat one or more neurological disorders such as mental health disorders e.g., mood disorders, anxiety disorders, eating disorders, substance-related disorders, and stress disorders e.g., post-traumatic stress disorder, learning disorders e.g., autism, and pain disorders e.g., neuropathic pain. In other forms, the dendrimer-hallucinogenic conjugates may be administered to a subject in need for stabilizing moods e.g., in bipolar disorder, reducing anxiety in anxiety disorders and reducing cancer related psychiatric distress. In yet other forms, the dendrimer is complexed to one or more hallucinogens to treat non-neural diseases such as autoimmune disorders.

Typically, an effective amount of dendrimer complexes including a combination of a dendrimer with one or more therapeutic, prophylactic, and/or diagnostic active agents are administered to an individual in need thereof. The dendrimers may also include a targeting agent, but as demonstrated by the examples, these are not required for delivery to injured tissue in the spinal cord, the brain, and related areas.

In some embodiments, the dendrimer complexes include an agent that is attached or conjugated to a dendrimer, which are capable of preferentially releasing the drug at the target receptor. The agent can be either covalently attached or intra-molecularly dispersed or encapsulated. The amount of dendrimer complexes administered to the subject is selected to deliver an effective amount to reduce, prevent, or otherwise alleviate one or more clinical or molecular symptoms of the disease or disorder to be treated compared to a control, for example, a subject treated with the active agent without dendrimer.

### B. Conditions and Diseases to be Treated

### 1. Site-Specific Targeting

The compositions and methods are designed to circumvent existing challenges in selective drug delivery to the central and peripheral nervous system. The compositions and methods may increase drug bioavailability in the central and peripheral nervous system by one or more of the following: (i) increasing drug density across brain barriers, particularly the blood-brain and blood-cerebrospinal fluid barriers, (ii) increasing drug solubility, (iii) facilitating target engagement i.e., increasing site-specific binding, (iv) improving drug pharmacokinetics, and (v) increasing intracerebral distribution. For example, in some forms, the compositions and methods permit selective delivery of compounds to the peripheral nervous system, thereby increasing the potential of the compositions to be used to selectively treat periphery-specific diseases and disorders, including but not limited to neuropathic pain, traumatic nerve injury, and inflammatory disorders.

### A. Improve Drug Permeability Across Barrier Interfaces

The dendrimer compositions and methods may improve the passage of the hallucinogen compounds across one or more of the barrier interfaces in the brain and nervous system, particularly the Blood-Brain Barrier (BBB), the CSF-blood barrier, and the blood-nerve barrier. These barrier interfaces typically protect neurons from blood-borne substances and help maintain water homeostasis and appropriate milieu for neuronal function in the blood. Due to the clinical significance of hallucinogens for the treatment of mental health disorders, the dendrimer compositions may be used to deliver hallucinogenic compounds with improved permeability across these barrier interfaces for site-specific targeting.

i. The Blood-Brain Barrier

The “Blood-Brain Barrier” (BBB) is a continuous endothelial membrane that, along with pericytes and other components of the neurovascular unit, limits the entry of toxins, pathogens, and blood cells to the brain. However, the BBB also represents an obstacle in the delivery of drugs to the central nervous system (CNS), in part because (1) delivering drugs intended for the brain via systemic routes may result in unacceptably high levels of drugs in the periphery; (2) the complex interplay of cells and molecules that contribute to the BBB's structure and function makes it challenging to determine drug permeability at the BBB, drug distribution in the brain, and target engagement in the brain.

Brain microvascular endothelial cells, pericytes, astrocytes, tight junctions, neurons, and basal membrane construct physically tight brain capillaries in the BBB. The brain capillary endothelial cells do not have fenestrations, which limits the diffusion of small molecules and proteins. Inter-endothelial junctions link the endothelial cells to a continuous barrier, severely restricting the penetration of water-soluble substances. Pericytes, astrocytes and basal membrane surround the endothelial cells and finally form the impermeable BBB. Additionally, efflux transporters are located in brain capillary endothelial cells, which are further obstacles against substances entering the brain. The permeability of the BBB is mainly controlled by inter-endothelial junctions that are protein complexes such as adherens junctions, tight junctions, and gap junctions. Adherens junctions primarily regulate the permeability of the endothelial barrier. Tight junctions play a vital role in sustaining the permeability barrier of epithelial and endothelial cells, which control tissue homeostasis. Gap junctions, composed of six connexin molecules, direct electric, and chemical communication between endothelial cells. Finally, instead of having a static structure, the components of the BBB continuously adapt in response to various physiological changes in the brain. The dendrimer compositions and methods overcome the aforementioned challenges and are suitable for delivering hallucinogen compounds across the blood-brain barrier via one or more of the above-described transport mechanisms.

Molecules cross the BBB by a paracellular pathway (between adjacent cells) or a transcellular pathway (through the cells). For the paracellular pathway, ions and solutes utilize concentration gradients to pass the BBB by passive diffusion. The transcellular pathway includes different mechanisms such as passive diffusion, receptor-mediated transport, and transcytosis.

The physicochemical factors that influence BBB permeability include molecular weight, charge, lipid solubility, surface activity and relative size of the molecule. BBB permeability can also be influenced by physiological factors such as efflux transporters, e.g., P-glycoprotein (P-gp), enzymatic activity, plasma protein binding and cerebral blood flow. Hydrophilic molecules such as proteins and peptides enter the brain through specific and saturable receptor-mediated transport mechanisms such as glucose transporter-1 (GLUT-1), insulin transporter and transferrin transporter. These endogenous transporters are expressed at the luminal and abluminal endothelial cell membranes. Among these transport mechanisms, receptor-mediated transcytosis has been extensively studied to deliver drugs into the brain. The dendrimer compositions and methods of the present application are suitable for delivering hallucinogenic compounds across the blood-brain barrier via one or more of the above-described mechanistic routes.

ii. The Blood-Nerve Barrier (BNB)

The blood-nerve barrier (BNB) defines the physiological space within which the axons, Schwann cells, and other associated cells of peripheral nerve function, thereby ensuring proper function of peripheral nerves, and maintenance of homeostasis of the endoneurial environment. The BNB consists of the endoneurial microvessels within the nerve fascicle and the investing perineurium. Tight junctions between endothelial cells and between pericytes in endoneurial vasculature isolate the endoneurium from the blood, thus preventing uncontrollable leakage of molecules and ions from the circulatory system to the peripheral nerves. In addition, a diffusion barrier exists within the perineurium formed by tight junctions between the neighboring perineurial cells and basement membranes surrounding each perineurial cell layer. The endoneurial capillaries and the perineurial passage are the restrictive barriers which separate the endoneurial extracellular environment of peripheral nerves from both the epineurial perifascicular space and the systemic circulation, thus protecting the endoneurial microenvironment from drastic concentration changes in the vascular and other extracellular spaces.

For drug targets located in peripheral nerves, the BNB can be problematic because of the potential to restrict or prevent drugs from reaching their site of action, thus negatively affecting drug efficacy. In addition, transporter expression profiles in peripheral nerves can be very different from those in the central nervous system. The dendrimer compositions of the present application may be used to improve permeability of hallucinogenic compounds across the BNB, thereby improving delivery of hallucinogenic compounds to peripheral nerve targets.

iii. The Blood-CSF Barrier

The composition may be used to improve delivery of hallucinogenic agents to target sites via the blood-cerebrospinal fluid barrier (blood-CSF barrier) and the ventricles. The choroid plexus is a vascular tissue found in all cerebral ventricles. The functional unit of the choroid plexus, composed of a capillary enveloped by a layer of differentiated ependymal epithelium. Unlike the capillaries that form the blood-brain barrier, choroid plexus capillaries are fenestrated and have no tight junctions. The endothelium, therefore, does not form a barrier to the movement of small molecules. Instead, the blood-CSF barrier at the choroid plexus is formed by the epithelial cells and the tight junctions that link them. The other part of the blood-CSF barrier is the arachnoid membrane, which envelops the brain. The cells of this membrane also are linked by tight junctions.

The CSF spaces and the cerebral structures adjacent to CSF compartments are pharmacological targets of interest in CNS diseases. For example, the subarachnoid, perivascular, or periventricular spaces are areas of pathogenic lymphocyte, monocyte, and neutrophil accumulation in neuroinflammatory disorders such as autoimmune disorders, eosinophilic inflammation, and/or asthma. Foci of B-cells detected in different CNS autoimmune diseases and producing potentially deleterious antibodies are thought to be mainly localized in leptomeninges. Therefore, in some forms, the dendrimer compositions may be used to deliver antidepressant and/or antipsychotic agents to areas of interest via the blood-CSF spaces connected with deep cervical lymph nodes for ameliorating or treating symptoms associated with neuroinflammatory disorders e.g., changes in cytokine response following stroke and allergy attacks.

In some forms, the dendrimer compositions may be used to deliver hallucinogenic agents to target sites for ameliorating or treating psychiatric and inflammatory symptoms associated with tumor development. Therefore, in some forms, the dendrimer compositions may be used to leverage pharmacological pressure from the CSF to achieve therapeutic concentrations of hallucinogenic compounds within the tumor microenvironment to reduce the inflammatory response and tumor development.

b. Improve Target-Specific Binding

The dendrimer compositions are suitable for delivering the hallucinogenic compounds with increased binding affinity and specificity to one or more receptors for modulation of the serotonin (5HT) receptors e.g., 5HT-1A, 5HT-2B, 5HT-2A, 5HT-2B, 5HT-2C, 5HT-3, 5HT-4, 5HT-6, and 5HT-7 receptors and/or one or more dopamine receptors e.g., dopamine D1 and D2 receptors. The dendrimer compositions may also be used to deliver the hallucinogenic compounds for direct or indirect modulation of one or more norepinephrine (NE) receptors e.g., α_{2A}-adrenergic receptor, α_{2B}-adrenergic receptor, α_{2C}-adrenergic receptor, β adrenergic receptor, monoamine transporters e.g., the serotonin reuptake transporter (SERT), dopamine transporter (DAT), and/or vesicular monoamine transporter (VMAT2). Additionally, hallucinogenic compounds that have affinity for and are suitable for binding one or more AMPA receptors, NMDA receptors, EGFR1 receptors, EGFR2 receptors, histamine (H1) receptors, GABA receptors, and trace amine-associated receptor 1 (TAAR1) may also be delivered using the dendrimer compositions described herein. Finally, the dendrimer compositions may be used to deliver the hallucinogenic agents with increased binding affinity and specificity to one or more receptors for modulation of one or more of the GLUT1, GLUT2, GLUT3, GLUT4, GLUT5, GLUT6, GLUT7, GLUT8, GLUT9, GLUT10, GLUT11, GLUT12, GLUT13, and GLUT14 transporters.

i. Improve Serotonin Receptor Binding

The dendrimer compositions may improve binding to one or both serotonin receptors to modulate signaling in a cell-specific and tissue-specific manner.

In the central nervous system (CNS), serotonin is almost exclusively produced in neurons originating in the raphe nuclei located in the midline of the brainstem. These serotonin-producing neurons form the largest and most complex efferent system in the human brain. The most caudal raphe innervates the spinal cord, while the more rostral raphe, the dorsal raphe nucleus, and the medial raphe nucleus, innervate much of the rest of the CNS by diffuse projections. Almost every cell in the brain is close to a serotonergic fiber, and nearly all behaviors as well as many other brain functions are regulated by serotonin.

Serotonin produces its effects through interactions with 13 serotonin G protein-coupled receptors (GPCRs). Serotonin receptors are prevalent throughout the body and regulate a range of diverse processes including but not limited to learning and memory, control of sleep/wake cycles, thermoregulation, appetite, sexual behavior in males and females, pain, motor activity, and aspects of autonomic function like arterial pressure and heart rate.

5-HT2Ars are broadly expressed in the cerebral cortex-especially in layers I and IV-V, the piriform and entorhinal cortex, the claustrum, endopiriform nucleus, and olfactory bulb/anterior olfactory nucleus, brainstem, as well as the limbic system and the basal ganglia; especially in the nucleus accumbens and caudate nucleus. Accordingly, dysfunction in the serotoninergic system is associated with several diseases and disorders e.g., anxiety and depressive disorders, migraine headaches, personality disorders, obsessive-compulsive disorders, drug addiction, and neurodegenerative disorders. Activation of the 5-HT2A receptor is necessary for the effects of the “classic” psychedelics like LSD, psilocin and mescaline, which act as full or partial agonists at this receptor and represent the three main classes of 5-HT2A agonists, the ergolines, tryptamines and phenethylamines, respectively. Therefore, in some embodiments, the dendrimer-hallucinogenic compositions can be used to activate 5-HT2A receptors to ameliorate symptoms associated with one of the above-mentioned disorders. For example, a dendrimer-R-DOI conjugate may be administered to a subject in need to reduce anti-inflammatory response associated with a neurodegenerative or anxiety disorder, optionally, via functional selectivity. Functional selectivity refers to the process by which different ligands induce slightly different conformations of the receptor to recruit different sets of effector pathways. In a second example, the compositions may activate 5-HT2A receptors located on the apical dendrites of pyramidal cells within regions of the prefrontal cortex to induce hallucinogenic activity. In a third example, the compositions may be used to deliver a classical psychedelic to a subject in need to modulate the activity of the receptor heterodimer 5-HT2A-mGlu2 and dopamine receptors to enhance PFC activity and ameliorate learning, memory and attention deficits.

The 5-HT2A receptor is also widely distributed in peripheral tissues and serotonergic dysregulation is also implicated in diseases in peripheral tissues, such as pulmonary hypertension, cancer of the bile duct, chronic kidney failure, and inflammatory bowel disease In some forms, the compositions may be used to modulate 5-HT2A receptor activity in immune-related tissues e.g., the spleen, thymus, and circulating lymphocytes. In some forms, the compositions are used to modulate the levels of 5-HT2A receptor protein in peripheral blood mononuclear cells (PBMCs), eosinophils, and T cells in order to regulate the innate and/or adaptive immune response. For example, the compositions may be administered to a subject in need to reduce inflammation and eosinophil infiltration due to allergy induced asthma.

ii. Improve Dopamine Receptor Binding

There are five types of dopamine receptors, which include D1, D2, D3, D4, and D5, with each receptor having a different function. The dendrimer compositions may improve binding to one or more dopamine receptors, preferably D1 and/or D2 receptors to modulate signaling in a cell-specific and tissue-specific manner. D1 receptors are implicated in memory, attention, impulse control, regulation of renal function, locomotion, and couple to G stimulatory sites and activate adenylyl cyclase. The activation of adenylyl cyclase leads to the production of the second messenger cAMP, which leads to the production of protein kinase A (PKA) which leads to further transcription in the nucleus. On the other hand, D2 receptors are implicated in locomotion, attention, sleep, memory, and learning, and couple to G inhibitory sites, which inhibit adenylyl cyclase and activate K+ channels.

Dopamine receptors are expressed in the central nervous system, specifically in the hippocampal dentate gyrus and subventricular zone. D1 receptors have high density in the striatum, nucleus accumbens, olfactory bulb, and substantia nigra. These receptors regulate the reward system, motor activity, memory, and learning. D1 receptors, along with stimulating adenyl cyclase, also activate phospholipase C, which leads to the induction of intracellular calcium release and activation of protein kinase C. Protein kinase C is a calcium-dependent protein kinase. Calcium also modulates neurotransmitter release by exocytosis. D2 receptors are expressed mainly in the striatum, as well as the external globus pallidus, core of nucleus accumbens, hippocampus, amygdala, and cerebral cortex, and modulate the postsynaptic receptor-medicated extrapyramidal activity. D2 receptors are important in the signaling for the survival of human dopamine neurons and neuronal development. Dopamine receptors are also expressed in the periphery, more prominently in kidney and vasculature. For example, in the kidney, D1 receptors inhibit Na/K ATPase through PKA and PKC pathways, thereby increasing electrolyte excretion and renal vasodilation.

Therefore, in some forms, the compositions may be used to deliver LSD alone or in combination with L-DOPA to modulate D1 and D2 receptor activity and ameliorate symptoms of Parkinson's-induced psychoses such as depression, anxiety, limb pain, fatigue, sleep disruption, and cognitive impairments.

iii. Improve Noradrenergic Receptor Binding

The dendrimer compositions may improve binding to one or both noradrenaline receptors to modulate signaling in a cell-specific and tissue-specific manner.

Norepinephrine, also known as noradrenaline, is a neurotransmitter of the brain that plays an essential role in the regulation of arousal, attention, cognitive function, and stress reactions. It also functions as a hormone peripherally as part of the sympathetic nervous system in the “fight or flight” response. During states of stress or anxiety, norepinephrine and epinephrine are released and bind to adrenergic receptors throughout the body which exert effects such as dilating pupils and bronchioles, increasing heart rate and constricting blood vessels, increasing renin secretion from the kidneys, and inhibiting peristalsis. The noradrenergic system plays a role in the pathogenesis of some significant neuropsychiatric disorders and has been an important pharmacologic target in various psychiatric, neurologic, and cardiopulmonary disorders.

The central noradrenergic system is composed of two primary ascending projections that originate from the brainstem: The dorsal noradrenergic bundle (DNB), and the ventral noradrenergic bundle (VNB). The DNB originates from A6 locus coeruleus, located in the dorsal pons, and is composed of primarily noradrenergic neurons. It functions as the predominant site of norepinephrine production in the central nervous system. It sends projections to innervate the cerebral cortex, hippocampus, and cerebellum exclusively and has projections that overlap with projections from the VNB to innervate areas of the amygdala, hypothalamus, and spinal cord. The VNB originates from nuclei in the pons and medulla and sends projections to innervate the amygdala, hypothalamus, and areas of the midbrain and medulla.

The sympathetic nervous system and neuroendocrine chromaffin cells (located in the adrenal medulla) are primarily responsible for the synthesis and exocytosis of norepinephrine and other catecholamines into the blood circulation. The hormones act on alpha- and beta-adrenergic receptors of smooth muscle cells and adipose tissue located throughout the body.

Following an action potential into the presynaptic terminal, voltage-gated calcium channels are stimulated and bring an influx of calcium from the extracellular to intracellular space. This influx causes norepinephrine (stored in vesicles) to bind the cell membrane and get released into the synaptic cleft via exocytosis. Norepinephrine can then go on to bind three main receptors: alpha1 (alpha-1), alpha-2, and beta receptors. These receptors classify as G-protein coupled receptors with either inhibitory or excitatory effects and different binding affinities to norepinephrine.

Alpha-1 receptors further subdivide into alpha-la, alpha-1b, and alpha-1d receptors. These receptors are located postsynaptically in regions of the brain including the locus coeruleus, olfactory bulb, cerebral cortex, dentate gyrus, amygdala, and thalamus. Alph-1 receptors have intermediate binding affinity to norepinephrine and couple to the Gq protein signaling pathway. In this pathway, phospholipase C (PLC) is activated to convert phosphatidylinositol 4,5-bisphosphate (PIP2) to inositol 1,4,5-trisphosphate (IP3) and diacylglycerol (DAG) on the cell membrane. IP3 is released to the cytosol and binds to transmembrane IP3 receptors located on the endoplasmic reticulum (ER) which functions as a calcium channel. When bound, the receptor undergoes a conformational change leading to the release of calcium from the ER to the cytosol. DAG remains in the cell membrane and positively regulates protein kinase C (PKC), which functions to phosphorylate other proteins. These combined effects produce excitatory cellular effects.

Alpha-2 receptors subdivide into alpha-2a, alpha-2b, and alpha-2c receptors. These receptors are located both presynaptically and postsynaptically in regions of the brain including locus coeruleus, amygdala, and hypothalamus. These receptors have the highest binding affinity to norepinephrine and couple to the Gi/o protein signaling pathway. In this pathway, cAMP levels are decreased thereby leading to decreased adenylyl cyclase activity, producing inhibitory cellular effects. Presynaptic noradrenergic terminals contain alpha-2 autoreceptors which prevent further release of norepinephrine.

Beta receptors subdivide into beta-1, beta-2, and beta-3 receptors. These receptors are in various regions of the brain, with beta-1 and beta-2 receptors being most prevalent in the cerebral cortex. These receptors have the lowest binding affinity to norepinephrine and couple to the Gs protein signaling pathway. In this pathway, cAMP levels increase leading to protein kinase A (PKA) activation which goes on to phosphorylate other proteins inside the cell and leads to excitatory cellular effects. Beta-2 receptors also couple to Gi protein signaling pathways. Beta-3 receptors are present in adipose tissue.

In the adrenal medulla, acetylcholine stimulates adrenaline and noradrenaline release. Acetylcholine binds to nicotinic receptors located on adrenal chromaffin cells, which generate action potentials sustained by voltage-gated sodium and potassium channels. This action potential triggers calcium influx into the cytosol, leading to norepinephrine vesicles binding to the cell membrane leading to the release of norepinephrine into the blood circulation where travel to bind alpha and beta receptors on smooth muscle and adipose cells.

Norepinephrine can be degraded intracellularly or in the synaptic cleft by the enzymes monoamine oxidase (MAO) or catechol-O-methyltransferase (COMT). MAO oxidizes norepinephrine while COMT metabolizes deaminated norepinephrine through O-methylation. MAO and COMT are found in adrenal chromaffin cells, while sympathetic nerves contain MAO only. COMT is found in all organs. The liver is responsible for the complete degradation of norepinephrine to vanillylmandelic acid (VMA).

### C. Conditions to be Treated

The compositions are suitable for treating one or more diseases, conditions, and injuries in the central and peripheral nervous system. The compositions can be used for treatment of a variety of diseases, disorders and injury including mental health disorders, gastrointestinal disorders, and/or treatment of other tissues where the nerves play a role in the disease or disorder. The compositions and methods are also suitable for prophylactic use. For example, the compositions may be administered to a patient in need thereof to ameliorate, treat or prevent symptoms associated with a variety of disorders, diseases, and conditions including but not limited to mental health and neurological disorders such as depression (including major depressive disorder, treatment-resistant depression, and post-partum depression), post-traumatic stress disorder, panic disorder, social anxiety disorder, anorexia nervosa, suicidal ideation, obsessive-compulsive disorder, premenstrual dysphoric disorder, anorexia, substance abuse disorders, epilepsy, autism spectrum disorders, attention-deficit hyperactivity disorder, schizophrenia, cluster headaches, migraines, seizures, fibromyalgia, narcolepsy, obesity, Alzheimer's disease, Tourette's syndrome, pain such as neuropathic pain and chronic pain, phobias, and cardiovascular diseases); pain disorders e.g., neuropathic pain, and/or gastrointestinal disorders. In other forms, the compositions may be administered to a subject in need for stabilizing moods e.g., in bipolar disorder, reducing anxiety in anxiety disorders and inflammation in Parkinson's Disease.

The dendrimer complex composition, preferably with a diameter under 20 nm and a hydroxyl group surface density at least 0.8 OH groups/nm², preferably under 10 nm and a hydroxyl group surface density of at least 1 OH groups/nm², more preferably under 6 nm and a hydroxyl group surface density of at least 1 OH groups/nm², and most preferably between 5 nm and a hydroxyl group surface density at least 1.5 OH groups/nm², delivering a therapeutic, prophylactic or diagnostic agent, selectively targets microglia and astrocytes, which play a key role in the pathogenesis of many disorders and conditions including neurodevelopmental, neurodegenerative diseases, neuropsychiatric disorders, and chronic pain. Thus, the dendrimer complexes are administered in a dosage unit amount effective to treat or alleviate conditions associated with the pathological conditions of the central and peripheral nervous system. For example, the dendrimer complexes are administered in a dosage unit amount effective to treat or alleviate conditions associated with pathological conditions that affects neurons microglia and astrocytes. Generally, by targeting these cells, the dendrimers deliver agent specifically to treat neuroinflammation.

In preferred embodiments, the compositions may include glucose or hydroxyl dendrimers 5 nm or smaller in diameter and conjugated to antidepressant, antipsychotic, or other agents that act via modulation of monoaminergic neurotransmission for the treatment of mental health and CNS disorders. In other preferred embodiments, the compositions may include glucose or hydroxyl dendrimers larger than 5 nm in diameter and conjugated to antidepressant, antipsychotic, or other agents that act via modulation of monoaminergic neurotransmission for the treatment of peripheral nervous system disorders.

a. Mental Health Disorders and Conditions

The compositions and methods are suitable for the treatment of a variety of mental health disorders and conditions including but not limited to affective or mood disorders, anxiety disorders, childhood disorders, eating disorders, personality disorders, and substance-related disorders.

i. Affective or Mood Disorders

Affective or mood disorders are described by marked disruptions in emotions (severe lows called depression or highs called hypomania or mania). These include bipolar disorder, cyclothymia, hypomania, major depressive disorder, disruptive mood dysregulation disorder, persistent depressive disorder, premenstrual dysphoric disorder, seasonal affective disorder, depression related to medical illness, depression induced by substance use or medication.

In some embodiments, the compositions and methods are suitable for the treatment of symptoms associated with depression, treatment resistant depression, and suicidal ideation. Up to 30% of people with depression fail to respond effectively to treatment with antidepressants, in part due to differences in biology between patients and the length of time it takes to respond to the drugs, resulting in low adherence to treatment regiments. Therefore, there is an urgent need to expand the repertoire of drugs available to people with depression.

In exemplary embodiments, the compositions are used to deliver psilocin, psilocybin and/or DMT to a subject suffering from treatment resistant depression to establish long-term behavioral outcomes such as improving coping strategies and enhancing cognitive function e.g., improving associative learning, a cognitive function commonly impaired by major depressive disorder (MDD).

ii. Anxiety Disorders

Anxiety disorders differ from normal feelings of nervousness or anxiousness and involve excessive fear or anxiety. Anxiety disorders include generalized anxiety disorder, panic disorder, social anxiety disorder, and various phobia-related disorders.

Generalized anxiety disorder (GAD) usually involves a persistent feeling of anxiety or dread, which can interfere with daily life. It is not the same as occasionally worrying about things or experiencing anxiety due to stressful life events. People living with GAD experience frequent anxiety for months, if not years. The compositions and methods are suitable for the treatment of one or more symptoms of GAD, including but not limited to restlessness, fatigue, difficulty concentrating, irritability, headaches, muscle aches, stomach aches, or unexplained pains, excessive worry, sleep issues e.g., difficulty falling or staying asleep.

Panic Disorder is an anxiety disorder characterized by unexpected and repeated episodes of intense fear accompanied by physical symptoms that may include chest pain, heart palpitations, shortness of breath, dizziness, or abdominal distress, or sense of losing control even when there is no clear danger or trigger. Individuals with panic disorders often worry about when the next attack will happen and actively try to prevent future attacks by avoiding places, situations, or behaviors they associate with panic attacks. Panic attacks can occur as frequently as several times a day or as rarely as a few times a year. The compositions and methods are suitable for the treatment of one or more symptoms of panic attacks including but not limited to heart palpitations, excess sweating, trembling, or tingling, chest pain, and difficulty controlling feelings e.g., feelings of impending doom and feelings of being out of control.

Social anxiety disorder is an intense, persistent fear of being watched and judged by others. For people with social anxiety disorder, the fear of social situations may feel so intense that it seems beyond their control. For some people, this fear may get in the way of going to work, attending school, or doing everyday things. The compositions and methods are suitable for the treatment of one or more symptoms of social anxiety disorders including but not limited to excess blushing, sweating, or trembling, heart palpitations, stomachaches, rigid body posture or speaking with an overly soft voice, and feelings of self-consciousness or fear of negative judgement.

A phobia is an intense fear of—or aversion to—specific objects or situations. Although it can be realistic to be anxious in some circumstances, the fear people with phobias feel are out of proportion to the actual danger caused by the situation or object. The compositions and methods are suitable for the treatment of one or more symptoms of phobias including but not limited to irrational or excessive worry about encountering the feared object or situation, immediate intense anxiety upon encountering the feared object or situation, and enduring unavoidable objects and situations with intense anxiety.

In an exemplary embodiment, the compositions may be used to deliver MDMA to a subject in need to ameliorate the symptoms associated with trauma-related disorders e.g., PTSD such as reducing pathological fear responses, social disconnection, and emotional numbing, and increasing trust and pro-social behaviors. In another exemplary embodiment, the compositions are suitable for delivering LSD to a subject in need for the treatment of obsessional neuroses, cancer-associated anxiety, and/or alcohol-use disorder.

iii. Eating Disorders

Eating disorders are serious and often fatal illnesses that are associated with severe disturbances in people's eating behaviors and related thoughts and emotions. Eating disorders include preoccupation with food, body weight, and shape. Common eating disorders include anorexia nervosa, bulimia nervosa, and binge-eating disorder.

Anorexia nervosa is a condition where people avoid food, severely restrict food, or eat very small quantities of only certain foods. They also may weigh themselves repeatedly. Even when dangerously underweight, they may see themselves as overweight. There are two subtypes of anorexia nervosa: a restrictive subtype and a binge-purge subtype. People with the restrictive subtype of anorexia nervosa severely limit the amount and type of food they consume. People with the binge-purge subtype of anorexia nervosa also greatly restrict the amount and type of food they consume. In addition, they may have binge-eating and purging episodes-eating large amounts of food in a short time followed by vomiting or using laxatives or diuretics to get rid of what was consumed. Symptoms of anorexia nervosa including but not limited to thinning of the bones (osteopenia or osteoporosis), mild anemia and muscle wasting and weakness, brittle hair and nails, dry and yellowish skin, growth of fine hair all over the body (lanugo), severe constipation, low blood pressure, slowed breathing and pulse, damage to the structure and function of the heart, brain damage, multiorgan failure, drop in internal body temperature, causing a person to feel cold all the time, lethargy, sluggishness, or feeling tired all the time, and infertility.

Bulimia nervosa is a condition where people have recurrent and frequent episodes of eating unusually large amounts of food and feeling a lack of control over these episodes. This binge-eating is followed by behavior that compensates for the overeating such as forced vomiting, excessive use of laxatives or diuretics, fasting, excessive exercise, or a combination of these behaviors. People with bulimia nervosa may be slightly underweight, normal weight, or over overweight. Symptoms of bulimia nervosa include chronically inflamed and sore throat, swollen salivary glands in the neck and jaw area, worn tooth enamel and increasingly sensitive and decaying teeth as a result of exposure to stomach acid, acid reflux disorder and other gastrointestinal problems, intestinal distress and irritation from laxative abuse, severe dehydration from purging of fluids, electrolyte imbalance (too low or too high levels of sodium, calcium, potassium, and other minerals) which can lead to stroke or heart attack.

In an exemplary embodiment, the compositions may be used to deliver psilocybin, LSD, and/or ayahuasca to a subject in need to (1) increase connectivity between neuronal networks, and create the potential to move beyond self-imposed limitations that are debilitating in the subject and/or (2) foster desirable brain states that might accelerate therapeutic processes e.g., increase neuroplasticity and neurogenesis, improve mood, decrease fear responses, and facilitate acceptance and empathy for self and others.

b. Neurological and Neurodegenerative Diseases

The compositions and methods are suitable for the treatment of symptoms associated with neurological and neurodegenerative diseases.

Neurodegenerative diseases are chronic progressive disorders of the nervous system that affect neurological and behavioral function and involve biochemical changes leading to distinct histopathologic and clinical syndromes (Hardy H, et al., Science 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

Neuroinflammation, mediated by activated microglia and astrocytes, is a major hallmark of various neurological disorders making it a potential therapeutic target (Hagberg, H et al., Annals of Neurology 2012, 71, 444; Vargas, D L et al., Annals of Neurology 2005, 57, 67; and Pardo, C A et al., International Review of Psychiatry 2005, 17, 485). Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, M L et al., Nat Rev Neurosci 2007, 8, 57). The delivery of therapeutics across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can also be used to deliver active agents for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder or peripheral symptoms resulting from a neurological or neurodegenerative disease or disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Multiple Sclerosis (MS), post-encephalitic dementia, cancer and chemotherapy-associated cognitive impairment and dementia, and depression-induced dementia and pseudodementia.

In an exemplary embodiment, the compositions may be used to deliver one or more psychedelic compounds to a subject in need to ameliorate the symptoms associated with a neurodegenerative disease such as stimulating neurogenesis, neuroprotection, and neuroplasticity, decreasing neural cell and oligodendrocyte cell loss, reducing oxidative stress and BBB disruption, and lowering inflammation and endoplasmic reticulum damage.

c. Pain

The compositions and methods are suitable for the treatment of neuropathic and/or non-neuropathic pain associated with various disorders, such as for example, complex regional pain syndrome, peripheral neuropathy, multiple sclerosis, and cancer-induced pain. For example, psilocybin and LSD can be effective in treating neuropathic (chronic) nerve pain. Chronic nerve pain, also known as neuropathic pain, is caused by nerve damage or other problems with the nerves, and is often unresponsive to regular painkillers, such as paracetamol. In some forms, the compositions can be used to deliver psilocybin and LSD to a subject in need for reducing the severity of headaches and migraines and extending the remission periods between headaches and migraines.

The mechanisms by which chronic pain develops are not completely understood but likely involve a complex interplay between somatic and visceral afferent input, peripheral and central sensitization, emotional state, and behavior and cognition. Distraction and changes in mood can have a powerful effect on the perception of pain. Therefore, in some embodiments, the compositions can be used to deliver psilocybin to a subject in need to reduce pain induced by cancer-associated anxiety and depression.

In some forms, the compositions can be used to treat cases of chronic pain that do not involve nerves (non-neuropathic pain). For example, the dendrimer compositions can be complexed with LSD or MDMA and used to treat chronic non-neuropathic pain. Conditions that cause non-neuropathic pain which may benefit from treatment with compositions include but are not limited to fibromyalgia, chronic back pain, and chronic neck pain. For example, the compositions may be used to deliver psilocybin or LSD to a subject in need to relieve symptoms associated with fibromyalgia, and/or chronic pelvic pain, including, but not limited to, reducing pain and stiffness in the muscles, abdomen, neck and/or back, fatigue, sleep disturbances, headaches, and migraines. The most important indications are major depressive disorder, treatment-resistant depression, post-traumatic stress disorder, panic disorder, social anxiety disorder, anorexia nervosa, suicidal ideation, obsessive-compulsive disorder, anorexia, substance abuse disorders, epilepsy, bi-polar disorder, autism spectrum disorders, attention-deficit hyperactivity disorder, schizophrenia, headaches, seizures, fibromyalgia, narcolepsy, obesity, Alzheimer's disease, and Tourette's syndrome.

### D. Dosage and Effective Amounts

Dosage and dosing regimens are dependent on the severity and location of the disorder or injury and/or methods of administration, as well as the specific agent being delivered. This can be determined by those skilled in the art.

In some embodiments, dosages are expressed in mg/kg, particularly when the expressed as an in vivo dosage of dendrimer-gene editing composition.

Typically, doses would be in the range from microgram/kg up to about 100 mg/kg of body weight. Dosages can be, for example 0.01 mg/kg to about 1,000 mg/kg, or 0.5 mg/kg to about 1,000 mg/kg, or 1 mg/kg to about 1,000 mg/kg, or about 10 mg/kg to about 500 mg/kg, or about 20 mg/kg to about 500 mg/kg per dose, or 20 mg/kg to about 100 mg/kg per dose, or 25 mg/kg to about 75 mg/kg per dose, or about 25, 30, 35, 40, 45, 50, 55, 60, 65, 70, or 75 mg/kg per dose.

Preferably, the compositions of dendrimer-gene editing agents do not target or otherwise genetically modify non-target or healthy cells not within or associated with the diseased tissue, or do so at a reduced level compared to cells associated with a disease or disorder such as a cancer and/or proliferative disorder. In this way, by-products and other side effects associated with the compositions are reduced. Therefore, in preferred embodiments, dendrimer compositions are administered in an amount that leads to an improvement, or enhancement, function in an individual with a disease or disorder, such as a cancer and/or proliferative disorder.

The actual effective amounts of the composition can vary according to factors including the specific agent administered, the particular composition formulated, the mode of administration, and the age, weight, condition of the subject being treated, as well as the route of administration and the disease or disorder. Generally, for intravenous injection or infusion, the dosage will be lower than for oral administration.

Dosage can vary and can be administered in one or more dose administrations daily, for one or several days. In some embodiments, dendrimer conjugation may increase the effectiveness and durability of the treatment, which may reduce the need for repeated administration to once per week, once per month, once per six months, once per year, or other longer-term dosing regimens. Some embodiments may be incorporated into drug delivery systems (e.g., implants, pumps, patches, creams, etc.) in order to provide controlled, sustained delivery in a manner that reduces the need for compliance and the potential for abuse. Guidance can be found in the literature for appropriate dosages for given classes of pharmaceutical products. Optimal dosing schedules can be calculated from measurements of drug accumulation in the body of the subject or patient. Persons of ordinary skill in the art can easily determine optimum dosages, dosing methodologies and repetition rates. Optimum dosages can vary depending on the relative potency of individual pharmaceutical compositions, and can generally be estimated based the effective dosages in in vitro and in vivo animal models. Notably, in some cases it may be ideal to minimize or prevent hallucinations associated with psychedelic compounds. This may be achieved via peripheral confinement of dendrimer compositions or via controlled and/or sustained activity of the dendrimer compositions.

Dosage forms of the pharmaceutical composition including the dendrimer compositions are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose.

In general, the timing and frequency of administration will be adjusted to balance the efficacy of a given treatment or diagnostic schedule with the side effects of the given delivery system.

In some embodiments, dosages are administered daily, biweekly, weekly, every two weeks or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

It will be understood by those of ordinary skill that a dosing regimen can be any length of time sufficient to treat the disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

### E. Controls

The therapeutic result of the composition including one or more gene editing compositions associated with or conjugated to a dendrimer can be compared to a control. Suitable controls are known in the art and include, for example, an untreated subject, or a placebo-treated subject. A typical control is a comparison of a condition or symptom of a subject prior to and after administration of glucose dendrimer compositions. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art. In some embodiments, an untreated control subject suffers from the same disease or condition as the treated subject.

In some embodiments, a control includes an equivalent amount of gene editing compositions delivered alone, or bound to dendrimers without glucose-based branching units such as dendrimers of a similar generation, molecular weight, and/or surface group density (e.g., hydroxyl groups).

### VI. Kits

The compositions can be packaged in kit. The kit can include a single dose or a plurality of doses of a composition including one or more psychedelic drugs associated with or conjugated to a dendrimer (e.g., one or more hydroxyl-terminated PAMAM dendrimers or glucose dendrimers as described in the Examples), and instructions for administering the compositions. Specifically, the instructions direct that an effective amount of the dendrimer composition be administered to an individual with a particular disease/disorder as indicated. The composition can be formulated as described above with reference to a particular treatment method and can be packaged in any convenient manner.

The present invention will be further understood by reference to the following non-limiting examples.

### EXAMPLES

### Example 1: Synthesis of hydroxyl-polyamidoamine (PAMAM-OH) Dendrimer Psychedelic Conjugate

The synthesis of PAMAM-OH-psychedelic conjugates was achieved via a variety of linking chemistries and linkers (both cleavable and non-cleavable). Briefly, the surface hydroxyl groups on PAMAM-OH are modified with a linker to bring a complementary group on the surface that can further react with the complimentary group on the drug linker. The drug compound is modified by the linker to bring a complimentary functional group for reacting with the dendrimer-linker. The linker on the drug is attached by cleavable or non-cleavable linkages. Examples of cleavable linkages include, esterase sensitive ester bond, glutathione sensitive disulfide bond, phosphatase-sensitive phosphodiester bond, triglycyl peptide linker (CX) capable of lysosomal release, and acid cleavable hydrazine linkage. The examples of non-cleavable linkages include ether, amino alkyl, or amide bonds. The linkers can be amino-acids, peptides, polyethylene glycol (n=2-15), or hydrocarbon chain.

1. Synthesis of Dendrimer-Psilocin Conjugate with a Cleavable Ester Linkage

The dendrimer-psilocin conjugate was prepared with a cleavable ester according to the reactions illustrated in FIGS. 1A and 1B. FIGS. 1A and 1B are schematics showing a stepwise synthetic route for conjugating psilocin to the dendrimer using click chemistry.

2. Synthesis of Dendrimer-Psilocin Analog with a Non-Cleavable Amide Linkage

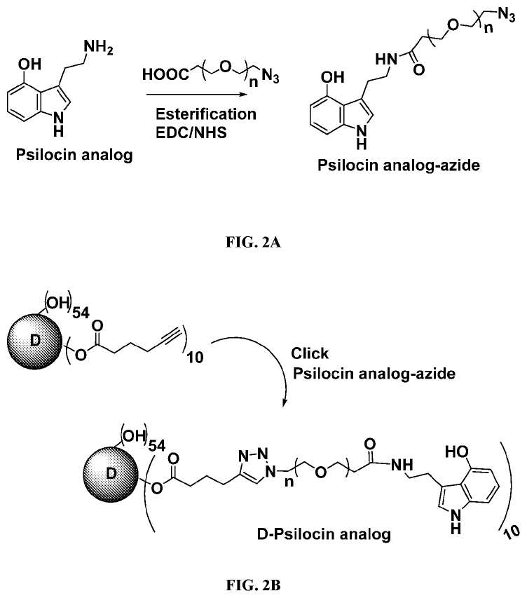

The dendrimer-psilocin conjugate was synthesized with a non-cleavable amide linkage according to the reaction illustrated in FIGS. 2A and 2B. FIGS. 2A and 2B are schematics showing a stepwise synthetic route for conjugating psilocin analog to the dendrimer using click chemistry.

3. Synthesis of Dendrimer-Ketamine with a Non-Cleavable Amino-Alkyl Linkage

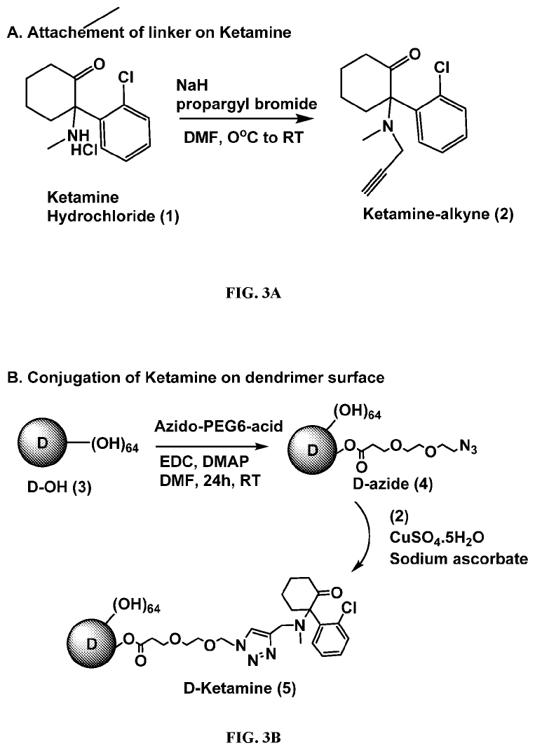

The dendrimer-ketamine conjugate was synthesized with a non-cleavable amino-alkyl linkage according to the reaction illustrated in FIGS. 3A and 3B. FIGS. 3A and 3B are schematics showing an exemplary synthetic route for conjugating ketamine to the dendrimer using copper-catalyzed alkyne azide click chemistry (FIG. 3B).

4. Synthesis of Dendrimer-DMT Analog with a Non-Cleavable Amide Linkage

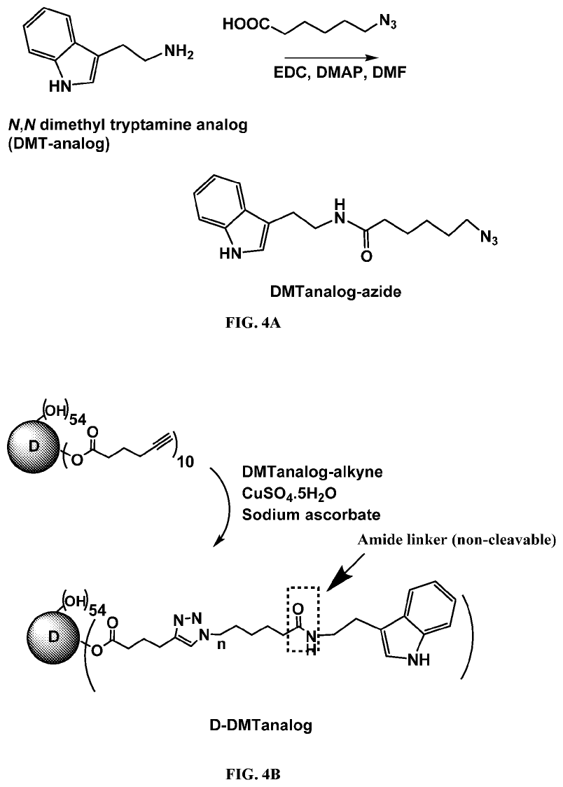

The dendrimer-N,N-Dimethyl tryptamine (dendrimer-DMT) conjugate was synthesized with a non-cleavable amide linkage according to the reaction illustrated in FIGS. 4A and 4B. FIGS. 4A and 4B are schematics showing an exemplary stepwise synthetic route for conjugating DMT to the dendrimer using copper-catalyzed alkyne azide click chemistry.

5. Synthesis of Dendrimer-DMT Analog with a Non-Cleavable Amino-Alkyl Linkage

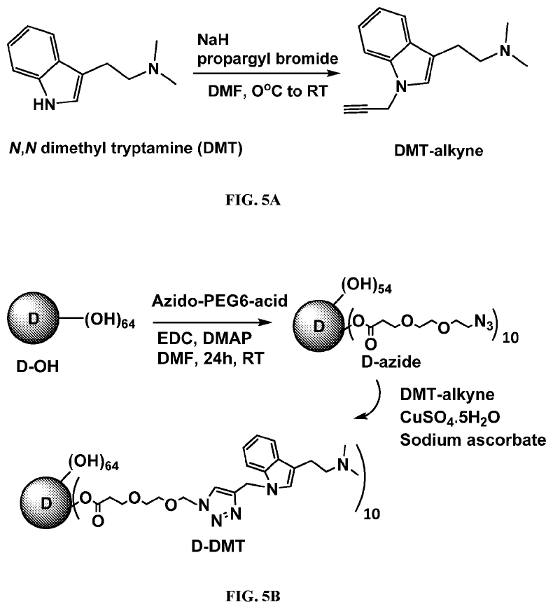

The dendrimer-DMT conjugate was also synthesized with a non-cleavable amino-alkyl linkage according to the reactions illustrated in FIGS. 5A and 5B. FIGS. 5A and 5B are schematics showing an exemplary stepwise synthetic route for conjugating DMT to the dendrimer using copper-catalyzed alkyne azide click chemistry.

6. Synthesis of Dendrimer-LSD with a Non-Cleavable Amino-Alkyl Linkage

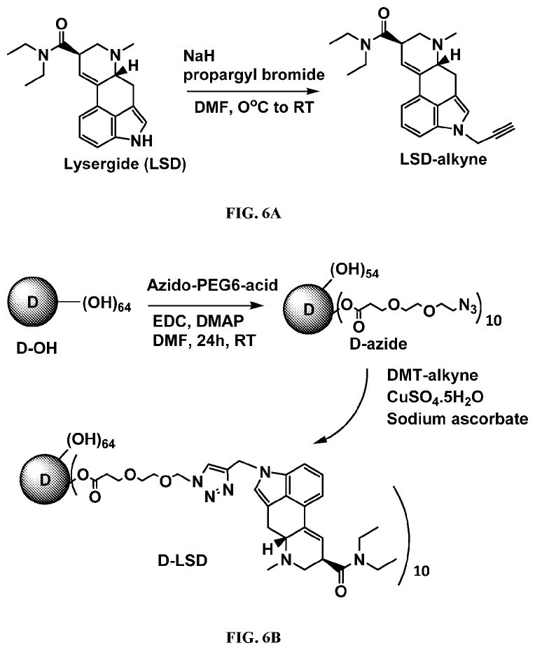

The dendrimer-lysergic acid diethylamide (dendrimer-LSD) conjugate was synthesized with a non-cleavable amino-alkyl linkage according to the reaction illustrated in FIGS. 6A and 6B. FIGS. 6A and 6B are schematics showing an exemplary stepwise synthetic route for conjugating LSD to the dendrimer using copper-catalyzed alkyne azide click chemistry.

### Conclusion

The dendrimer conjugation will provide site specific-targeting by directing the drugs to the target site, decreasing the dose, enhancing efficacy, and reducing side-effects related to free drugs. The PAMAM-OH platform has a very high-water solubility (>300 mg/mL). Most of the psychedelics have poor aqueous solubility in the ug/mL range. The dendrimer conjugation will improve the water solubility 10-100 fold compared to free drug. The sustained intracellular release at the target will avoid the systemic and dose-related side-effects of free psychedelic drugs. Dendrimer conjugation can significantly reduce the time required for onset of drug activity.

### Example 2: Synthesis of Glucose Dendrimer (GD) Psychedelic Conjugates

The synthesis of glucose dendrimer-psychedelic conjugates is achieved using a combination of linking chemistries and linkers (both cleavable and non-cleavable). Briefly, the surface hydroxyl groups on glucose dendrimers are modified with a linker facilitate the reaction of a complementary group on the surface with the complimentary group on the drug linker. The drug compound is also modified by a linker to facilitate a reaction between a complimentary functional group and a dendrimer-linker. The linker on the drug compound is attached via cleavable or non-cleavable linkages. The examples of cleavable linkages include esterase sensitive ester bond, glutathione sensitive disulfide bond, phosphatase-sensitive phosphodiester bond, triglycyl peptide linker (CX) capable of lysosomal release, and acid cleavable hydrazine linkage. The examples of non-cleavable linkages include ether or amide bonds. The linkers can be amino-acids, peptides, polyethylene glycol (n=2-15), or hydrocarbon chains.

1. Synthesis of Dendrimer-Psilocin with a Cleavable Ester Linkage

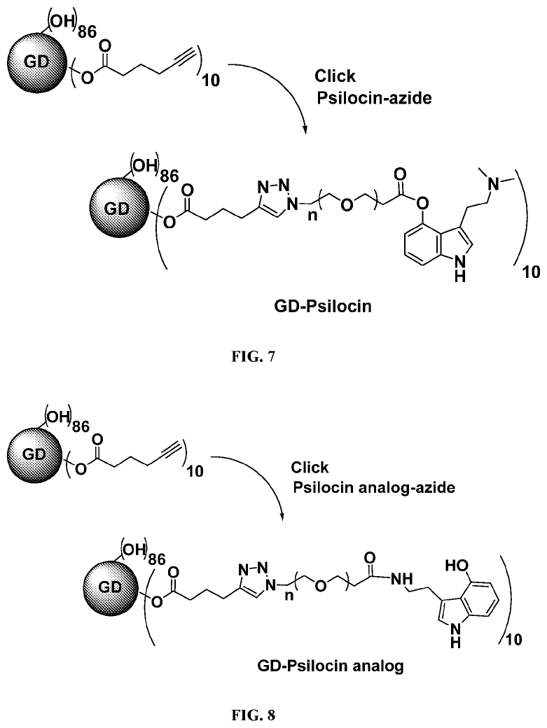

The glucose dendrimer-psilocin conjugate was prepared with a cleavable ester linkage according to the reactions illustrated in FIG. 7. The procedure for the synthesis of psilocin-azide compound is shown in FIG. 1A. The synthesis of glucose dendrimer and psilocin conjugate is achieved by the partial modification of OH groups of glucose dendrimers with a complimentary group on the surface of the glucose dendrimer which is reacted with a complimentary linker containing azide connected to the psilocin to generate the glucose dendrimer-psilocin conjugate (FIG. 7).

2. Synthesis of Dendrimer-Psilocin Analog Conjugate with a Non-Cleavable Amide Linkage

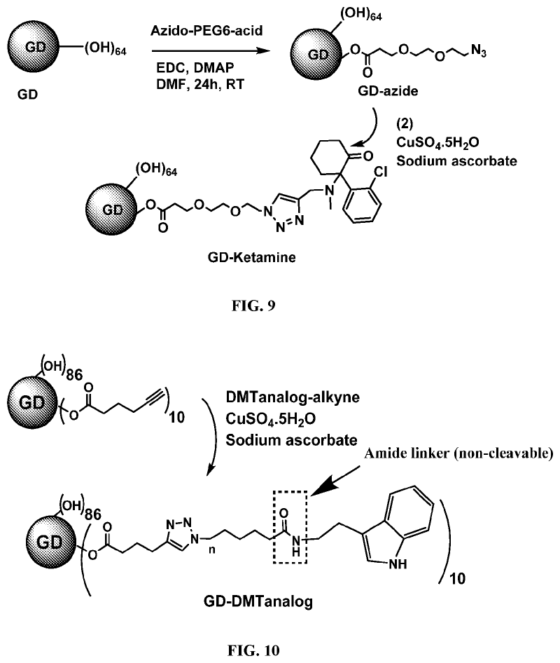

The glucose dendrimer-psilocin conjugate was prepared with a non-cleavable amide linkage according to the reactions illustrated in FIG. 8. The procedure for the synthesis of psilocin analog-azide compound is shown in FIG. 2A. The synthesis of glucose dendrimer and psilocin analog conjugate is achieved by the partial modification of OH groups of glucose dendrimers with a complimentary group on the surface of the glucose dendrimer which is reacted with a complimentary linker containing azide connected to the psilocin analog to generate the glucose dendrimer-psilocin conjugate (FIG. 8).

3. Synthesis of Dendrimer-Ketamine with a Non-Cleavable Amino-Alkyl Linkage

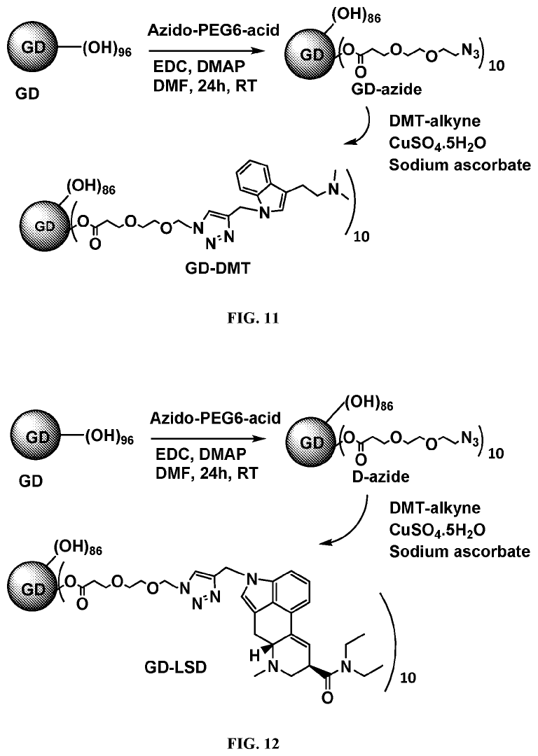

The glucose dendrimer-ketamine conjugate was prepared with a non-cleavable amino-alkyl linkage according to the reactions illustrated in FIG. 9. Ketamine hydrochloride (1) was first modified with an alkyne as shown in FIG. 3A. The exemplary synthesis route of glucose dendrimer and ketamine conjugate is shown in FIG. 9.

4. Synthesis of Dendrimer-DMT Analog with a Non-Cleavable Amide Linkage

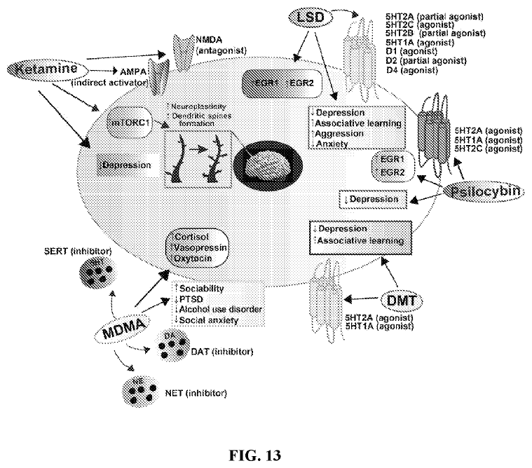

The glucose dendrimer-DMT conjugate was prepared with a non-cleavable amide linkage according to the reactions illustrated in FIG. 10. N,N dimethyl tryptamine analog (DMT analog) was first conjugated to a linker with an azide moiety via an amide linkage (FIG. 4A). The exemplary synthesis route of glucose dendrimer and DMT conjugate is shown in FIG. 10.

5. Synthesis of Dendrimer-DMT with a Non-Cleavable Amino-Alkyl Linkage

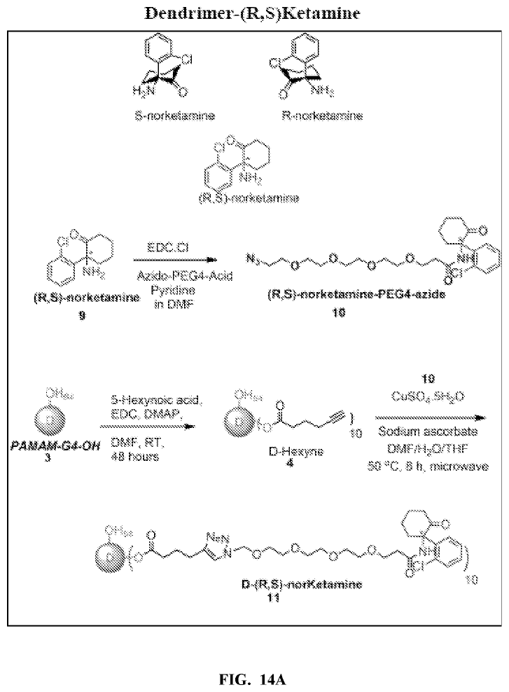

The glucose dendrimer-DMT conjugate was prepared with a non-cleavable amino-alkyl linkage according to the reactions illustrated in FIG. 11. N,N dimethyl tryptamine analog (DMT analog) is first modified with an alkyne group as shown in FIG. 5A. The exemplary synthesis route of glucose dendrimer and DMT conjugate is shown in FIG. 11.

6. Synthesis of Glucose Dendrimer-LSD with a Non-Cleavable Amino-Alkyl Linkage

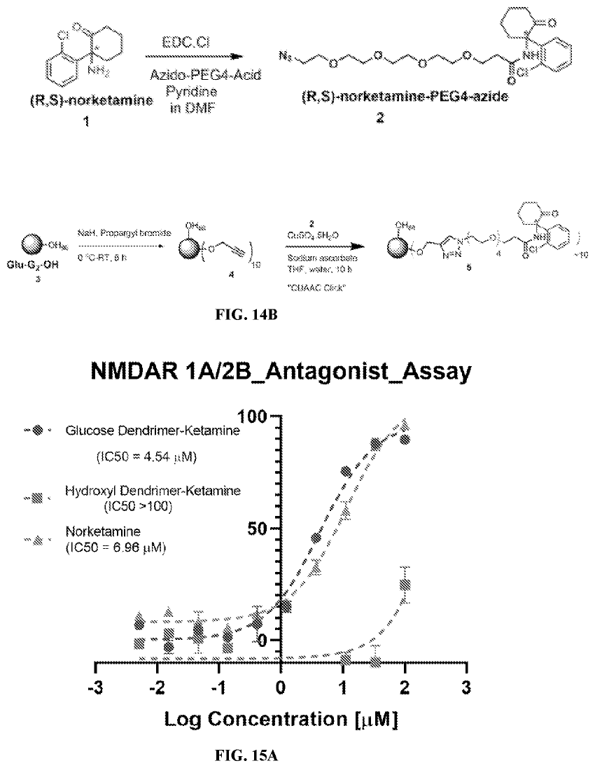

The glucose dendrimer-Lysergic acid diethylamide (LSD) conjugate was prepared with a non-cleavable amino-alkyl linkage according to the reactions illustrated in FIG. 12. LSD was first modified with an alkyne group as shown in FIG. 6A. The exemplary synthesis route of glucose dendrimer and LSD conjugate is shown in FIG. 12.

### Conclusion

The dendrimer conjugation provides site specific targeting by directing the drugs to the target site, decreasing the dose, enhancing efficacy, and reducing side-effects related to free drugs. The glucose dendrimer platform has a very high water solubility (>500 mg/mL). Most of the psychedelics have poor aqueous solubility in the ug/mL range. The dendrimer conjugation will improve the water solubility 10-100 fold compared to an unconjugated free drug. The sustained intracellular release at the target will avoid the systemic and dose-related side-effects of free psychedelic drugs. Dendrimer conjugation can significantly reduce the time required for onset of drug activity.

The compositions can deliver drugs to receptors on specific cells (neuronal cells, glial cells, macrophages), including targets on their surface and inside them. These formulations can enhance the effectiveness of these drugs enabling lower doses, lead to new mechanistic insights, reduce side effects, improve solubility, formulation, pharmacokinetics.

Collectively, dendrimer-based psychedelic agents will significantly improve the safety, efficacy, reproducibility, and ease of implementation of these molecules.

### Example 3: Synthesis of Dendrimer Conjugates and Binding Properties

New chemical entities of psychedelic drugs and norketamine with hydroxyl-terminated PAMAM, and glucose dendrimers have been synthesized, and studies conducted to determine in vivo efficacy and targeting data with norketamine/ketamine, and binding affinity data for D-tryptamine.

### Material and Methods

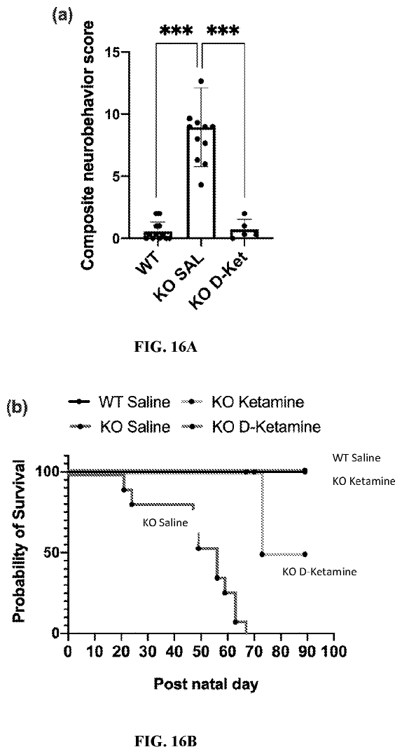

FIG. 14A is a schematic of the synthesis of a PAMAM dendrimer-norketamine conjugate. FIG. 14B is a schematic of the synthesis of a Glucose dendrimer-norketamine conjugate.

Unless stated otherwise, reactions were performed in flame dried glassware under a positive pressure of nitrogen using dry solvents. Commercial grade reagents and anhydrous solvents were purchased from chemical suppliers and used without further purification. 1-Ethyl-3-(3-dimethylaminopropyl) carbodiimide (EDC·HCl), N, N-diisopropylethylamine (DIPEA), 4-(dimethylamino)pyridine (DMAP) trifluoracetic acid (TFA), anhydrous dichloromethane (DCM), N,N′-dimethylformamide (DMF) were purchased from Sigma-Aldrich (St. Louis, MO, USA). Cyanine 5 (Cy5)-mono-NHS ester was purchased from Amersham Bioscience-GE Healthcare. Deuterated solvents dimethylsulfoxide (DMSO-d6), water (D₂O), and Chloroform (CDCl₃) were purchased from Cambridge Isotope Laboratories Inc. (Andover, MA). Ethylenediamine-core polyamidoamine (PAMAM) dendrimer, generation 4.0, hydroxy surface (G4-OH; diagnostic grade; consisting of 64 hydroxyl end-groups), methanol solution (13.75% w/w) was purchased from Dendritech Inc. (Midland, MI, USA). Dialysis membranes were purchased from Spectrum Laboratories Inc. (Rancho Dominguez, CA, USA). Hu308, Tryptamine, 1-(2-amino-1-(4-methoxyphenyl)ethyl) cyclohexanol, Nor-ketamine, 5-hydroxy tryptamine, psilocybin analog, psilocyn analog and cannabidiol drugs were purchased from Cayman Chemicals.

### Synthesis of Hydroxyl-PAMAM Dendrimer Drug Conjugates.

The PAMAM-G4-OH (D4-OH) dendrimer composed of about 64 terminal hydroxyl groups was used for the synthesis. After each synthetic step, the product was purified via dialysis in DMF for 24 h to eliminate small molecule impurities followed by water dialysis to remove DMF. 1H NMR (in DMSO-d6 and D20) and analytical HPLC were used to confirm the intermediates and final product formation and purity. The mono-functional D4-OH was functionalized with alkyne group by treatment of 5-hexynoic acid under standard esterification conditions using EDC·HCl and 4-DMAP in DMF for 36 h at room temperature to yield the D-hexyne bifunctional dendrimer. The number of alkyne groups on dendrimer surface was chosen to be kept at ~10-15 to maintain the overall water solubility of the conjugate. The crude product was dialyzed by 1 kDa membrane against ultrapure water for 24 h to remove low molecular weight impurities via selective diffusion across the semi-permeable dialysis membrane. The 1H NMR and analytical HPLC were used to confirm the product formation and purity of the intermediates and final products. These are shown in FIG. 12A-12C.

### Synthesis of D-Hexyne

A solution of PAMAM G4-OH 1 (10.00 g, 0.7 mmol) in DMF (50 mL) was treated with 5-Hexynoic acid (1.40 g, 12.6 mmol), DMAP (2.41 g, 12.6 mmol) and stirred at room temperature for 5 min. Then EDC·HCl (1.54 g, 12.6 mmol) was added in portions to the reaction mixture over the period of 5 min. The reaction mixture was stirred at room temperature for 36 h. The crude product was transferred to 1 kD MW cut-off cellulose dialysis tubing and dialyzed against DMF 12 h followed by water for 24 h. The aqueous layer was frozen and lyophilized to yield D-hexyne as a hygroscopic white solid (75% yield). ¹H NMR (500 MHz, DMSO-d6) 8.21-7.57 (m, internal amide H), 4.71 (s, GABA amide H, 50H), 4.01 (t, 22-24 CH2), 3.5-2.1 (m, dendrimer CH2) 1.71-1.59 (m, 22-24 CH2). HPLC C18 retention time 4 min: purity ~99%.

### Synthesis of Dendrimer-Drug Conjugate.

The solution of D-Hexyne and drug-azide in DMF (5 mL) was treated with copper sulfate pentahydrate (CuSO₄.5H₂O) and sodium ascorbate in water. The reaction mixture was stirred and heated for 10 h at 50° C. in a microwave synthesizer. On completion, the reaction mixture was dialyzed against DMF in 1KDa cut-off cellulose dialysis tubing. To this solution, EDTA (50 μL, 0.5M) solution was added for copper removal by chelation. The DMF dialysis was followed by water dialysis overnight. The drug loading is calculated by proton integration where peaks corresponding to dendrimer and drug are compared.

### G2-Glucose Dendrimer (GD2)-Drug Conjugate

Generation 2 glucose dendrimer (GD2) consists of 24 glucose molecules (96 surface hydroxyl groups) used for conjugation. Glucose dendrimers primarily are made of glucose moieties comprised of the central core of Di-pentaerythritol and one or more branching units of monosaccharide glucose molecules. Unlike hydroxyl-terminated PAMAM dendrimer, glucose dendrimers primarily are taken up by injured neurons and found to specifically target hyperexcitable neurons in both culture and in vivo mouse model.

### Synthesis and Characterization of Glucose Dendrimer (GD2).

The GD synthesis was begun by reacting hexapropargylated core with AB4, β-D-glucose-PEG4-azide building via click reaction to obtain generation 1 glucose dendrimer (GD1). The OH groups on GD1 were propargylated to obtain GD1-Acetylene24, which was reacted with β-D-glucose-PEG4-azide to obtain generation 2 (GD2) with 24 glucose moieties, providing 96 surface hydroxyl groups. Further the Cy5 fluorescent tag was attached on GD2 by propargylation of ~2-3 hydroxyl groups to bring alkyne containing GD2 dendrimer. The GD intermediates and final products were purified using dialysis and characterized using 1H NMR. The physicochemical properties of GD2 dendrimer were also evaluated (Table 2).

TABLE 2
Physiochemical Properties of GD2

| Size | 4.4 | nm |
|---|---|---|
| Zeta Potential | −6.0 | |
| Number of Glucose | 24 | |
| HPLC purity | >99% | |
| Molecular weight | 12800 | Da |

### Synthesis and Characterization of GD2-Drug Conjugates

The Norketamine, tryptamine, venlafaxine and Hu308 drugs were conjugated to the GD2-Hexynoic acid dendrimer using click chemistry strategy. The linker attached drug moieties were conjugated to glucose dendrimer using Cu (I) catalyzed click (CuAAC) reaction in the presence of catalytic amount of CuSO₄·5H₂O and sodium ascorbate to obtain GD2-drug conjugate. The traces of copper were removed by dialyzing with ethylenediaminetetraacetic acid (EDTA). The final GD2-drug conjugates were characterized by NMR and HPLC.

TABLE 3
Drugs and Conjugates thereof

| Active compound | Conjugatable analog |
|---|---|
| 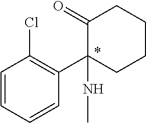 [SMILES: CNC1(c2ccccc2Cl)CCCCC1=O] | 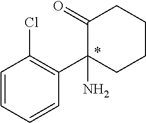 [SMILES: NC1(c2ccccc2Cl)CCCCC1=O] |
| (R,S)-ketamine | (R,S)-Norketamine |
| 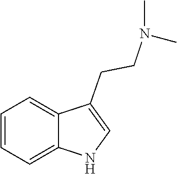 | 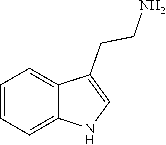 [SMILES: NCCc1c[nH]c2ccccc12] Tryptamine |
| N,N-DimethylTryptamine | |
| 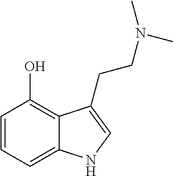 | 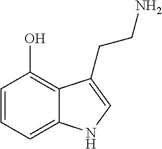 [SMILES: NCCc1c[nH]c2cccc(O)c12] |
| Psilocin | 4-Hydroxyltryptamine |
| 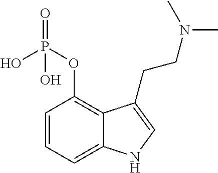 [SMILES: CN(C)CCc1c[nH]c2cccc(OP(=O)(O)O)c12] | 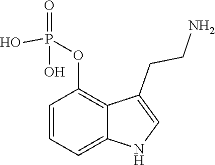 [SMILES: NCCc1c[nH]c2cccc(OP(=O)(O)O)c12] |
| Psilocybin | Norbeocystein |

TABLE 4
Physical properties of D-drug conjugates.

| | | Water | | |
|---|---|---|---|---|
| | | Solubility of | | |
| | Water | dendrimer- | | |
| | Solubility | drug | | |
| | of | conjugates | | |
| Dendrimer | Free | (based on free | Number of | |
| drug conjugate | drug* | drug) | attachments | DLS/nm |
| D-Norketamine | 0.178 | 30 | ~9-10 | 3.9 ± 0.1 |
| | mg/mL | mg/mL | | |
| D-Tryptamine | 1.34 | 10 | ~10 | 4.5 ± 0.1 |
| | mg/mL | mg/mL | | |
| D-5HT | 2.5 | 10 | ~3-4 | 4.0 ± 0.2 |
| | mg/mL | mg/mL | | |

*https://go.drugbank.com/metabolites/DBMET00189

### Example 4: Binding Assays of Dendrimer-Ketamine Conjugates

### Materials and Methods

### Human Serotonin 5-HT2A Receptor (Agonist Radioligand):

Purpose: Evaluation of the affinity of compounds for the human 5-HT2A receptor in transfected HEK-293 cells determined in a radioligand binding assay.

Experimental protocol: Cell membrane homogenates (30 μg protein) are incubated for 60 min at 22° C. with 0.1 nM [125I] DOI in the absence or presence of the test compound in a buffer containing 50 mM Tris-HCl (pH 7.4), 5 mM MgCl2, 10 μM pargyline and 0.1% ascorbic acid.

Nonspecific binding is determined in the presence of 1 μM DOI. Following incubation, the samples are filtered rapidly under vacuum through glass fiber filters (GF/B, Packard) presoaked with 0.3% PEI and rinsed several times with ice-cold 50 mM Tris-HCl using a 96-sample cell harvester (Unifilter, Packard). The filters are dried then counted for radioactivity in a scintillation counter (Topcount, Packard) using a scintillation cocktail

### (Microscint 0, Packard).

The results are expressed as a percent inhibition of the control radioligand specific binding.

The standard reference compound is DOI, which is tested in each experiment at several concentrations to obtain a competition curve from which its IC50 is calculated.

See Bryant, et al. (1996), a novel class of 5-HT2A receptor antagonist: aryl aminoguanidines, Life Sci., 15:1259.

Delta (DOP) Human Opioid GPCR Cell Based Antagonist cAMP Assay:

Purpose Evaluation of the potency (IC50) and efficacy (Max response) of compounds for the human delta (DOP) receptor in stably transfected CHO-K1 cells. Assay principle is cAMP cell-based assay.

Experimental protocol Cells were seeded in a total volume of 20 μL into white walled, 384-well microplates and incubated at 37° C. overnight. Prior to testing cell plating media was exchanged with 10 μL of Assay buffer (HBSS+10 mM HEPES)

Briefly, intermediate dilution of sample stocks was performed to generate 4× sample in assay buffer. 5 μL of 4× sample was added to cells and incubated at 37° C. for 30 minutes. 5 uL of 4×EC80 in 4× forskolin reagent was added and cells incubated for 37° C. for 30 minutes. Final assay vehicle concentration was 1%. The results are expressed as a percent inhibition of the control ligand.

### General Information

Assay volume and format: 10 μl in 384-well plate
Compound addition: 5 ul of 4× compound.
Maximum tolerable DMSO concentration: Customer compounds (when in DM diluted as [100×] solution in solvent 1%
Assay Temperature: 37° C.
Incubation time: 30 mins
Forskolin Concentration: 20 μM
   TA1 Human Trace Amine GPCR Cell Based Agonist cAMP Assay

Purpose: Evaluation of the potency (EC50) and efficacy (Max response) of compounds for the human TAI receptor in stably transfected CHO-K1 cells determined in a GPCR cell based cAMP assay.

Experimental protocol: Cells were seeded in a total volume of 20 uL into white walled, 384-well microplates and incubated at 37° C. overnight prior to testing.

Prior to testing cell plating media was exchanged with 15 μL of Assay buffer (HBSS+10 mM HEPES). Briefly, intermediate dilution of sample stocks was performed to generate 4× sample in assay buffer. 5 μL of 4× was added to cells and incubated at 37° C. for 30 minutes. Final assay vehicle concentration was 1%.

The results are expressed as a percent efficacy relative to the maximum response of the control ligand.
General Information
Assay volume and format: 20 ul in 384-well plate
Compound addition: 5 ul of 4× compound. Customer compounds (when in DMSO) are diluted as [100×] solution in solvent
Maximum tolerable DMSO 1%
concentration:
Assay Temperature: 37° C.
Incubation time: 30 mins

### Results

TABLE 5
Results of Binding Assays

| | NMDAR | Opiate μ | Opiate k | Sigma 2 |
|---|---|---|---|---|
| NorKetamine | 0.33 μM | Not active | 667 μM | 246 μM |
| Glucose Dendrimer- | Not active | 91 μM | 143 μM | 147 μM |
| Ketamine | | | | |
| Hydroxyl-terminated | 1.7 μM | 64 μM | 98 μM | 68 μM |
| PAMAM Dendrimer- | | | | |
| Ketamine | | | | |

| | | | | NMDAR |
|---|---|---|---|---|
| | | | | 1A/2B |
| | | Opiate | | Ion |
| | DRD2L | delta1R | 5HT1AR | channel |
| | Agonist | (antagonist) | agonist | blocker |
| NorKetamine | Not active | Not active | | 6.9 μM |
| Glucose Dendrimer- | 13.07 μM | Not active | 23.13 μM | 4.6 μM |
| Ketamine | | | | |
| Hydroxyl-terminated | 4.3 μM | Not active | Not active | Not active |
| PAMAM Dendrimer- | | | | |
| Ketamine | | | | |

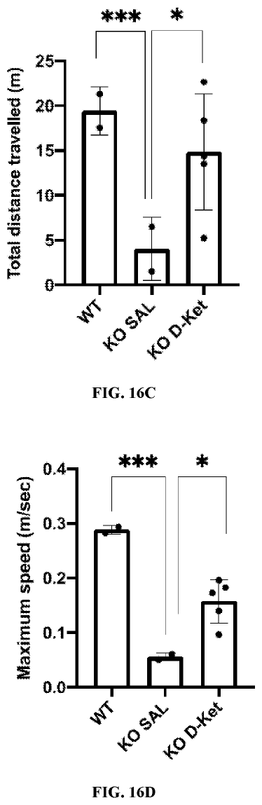

FIG. 15A is a graph of an NMDAR 1A/2B antagonist assay for glucose dendrimer-ketamine (IC50=4.54 μM), hydroxyl dendrimer-ketamine (IC50>100), and norketamine (IC50=6.96 μM). FIG. 15B is the % binding efficacy of the log concentration of compound in micromolar in a D2L human dopamine GPCR cell based agonist cAMP assay. Norketamine (solid circle), glucose dendrimer-ketamine EC50=13.08 micromolar (open circle), and hydroxyl dendrimer-ketamine EC50=4.263 micromolar (triangle). FIG. 15C is the % efficacy of the log concentration of ketamine in micromolar in the TAI human trace amine GPCR cell based agonist CAMP assay. Norketamine (solid circle), glucose dendrimer-ketamine EC50=13.08 micromolar (open circle), and hydroxyl dendrimer-ketamine EC50=4.263 micromolar (triangle).

Significant efficacy data was obtained with a clinically important drug that is derivative of ketamine (norketamine). Norketamine has been undergoing trials for depression and addiction. The hydroxyl dendrimer-drug target these receptors in microglia/macrophages, whereas glucose dendrimer-drug target these receptors both in neurons and microglia.

The conjugates can be active with or without releasing the drug. Binding affinities measure the activity of the intact conjugates. Typically, conjugates of psychedelic drugs are prepared in the non-releasing form (e.g. tryptamine, psilocin, psilocybin, ketamine), and are intended to be active in the intact form. This enables the intact conjugates to be released through the kidney with no toxicity from drug release. Alternatively, they can be designed to release through analogs.

Both GD-ketamine and HD-ketamine show effects that are unique and should be beneficial for neuropsychiatric conditions. Both dendrimer-ketamine conjugates show greater binding than nor-ketamine for opiate u, opiate k and sigma 2 receptors, indicating that dendrimer conjugation provides positive benefits to binding these receptors. Norketamine does not bind Opioid u receptors (Ki for GD-ket is 53 μM and 40 μM for HD-ket). Ki for opioid k is 381 μM for norketamine while it is about 4 fold better for GD-ket (Ki=82 μM) and ~7 fold better for HD-Ket (Ki=556 μm). The anti-depressant effects of ketamine are thought to be mediated through the opioid k receptors and dendrimer binding increases the affinity for those receptors.

Improved binding upon dendrimer conjugation is also seen with sigma 2 receptors. The greater ability of HD ketamine to bind Sigma 2 receptors indicates greater neuroprotective effects. The mechanism of neuroprotection may be related to increased NGF and BDNF production. Sigma 2 receptor activation can also be beneficial in neuropsychiatric conditions such as schizophrenia and psychosis. Decreased anxiety and greater anti-depressant effects can be seen with sigma 2 receptor targeting. Ketamine binds sigma2 receptors at mM concentrations. (Pergolizi, 2023; Bonaventura, 2021).

The ability to bind dopamine 2 receptors is seen only with the dendrimer-conjugated ketamine. Free norketamine does not appear to bind Dopamine 2 receptors. Similarly, ketamine has also not shown any affinity for dopamine 2 receptors. The dopaminergic effects of ketamine are believed to be indirect effects. However, it was shown that glucose dendrimer-ketamine has an RC50 of 13.07 UM at D2 receptors while hydroxyl dendrimer-ketamine is more effective with an RC50 of 4.3 μM. The anti-depressant and neuroprotective effects of ketamine are mostly mediated through these and the dopamine receptors. Ketamine, norketamine and metabolites of ketamine do not appear to have a direct effect on dopamine receptors are do not bind dopamine receptors. The increased activity seen at the D2 receptors on the functional assay indicates that dendrimer-ketamine will be more effective as an anti-depressant.

GD-ketamine and HD-ketamine show different features. Binding with hydroxyl dendrimer (HD) as compared to glucose dendrimer (GD) appears to change the function of ketamine, which is highly expected. HD-ketamine does not demonstrate NMDAR 1A/2B ion channel blockage while GD ketamine does demonstrate that the type of dendrimer that is bound to ketamine is critical for differences in function between them. GD-ketamine also shows agonist activity against 5HT1A receptors which is not seen with HD-ketamine.

These results indicate that dendrimer conjugation produces unexpected benefits to free drugs on receptor binding and is dependent on the dendrimer structure. The microglial targeting of hydroxyl dendrimer and the additional neuronal targeting of the glucose dendrimers bring unexpected results with clinical significance.

The conjugates are targeted to specific receptors on whatever cells they may be on. This includes neurons, microglia, macrophages and other cells. These receptors may be anywhere in the brain. Examples include serotonin receptors such as 5HT1A, 5HT2A, NMDA etc. Serotonin receptors can also be found in microglia. The compounds bind to and act on these receptors. The ‘intrinsic cellular targeting’ of these dendrimers (hydroxyl dendrimer-microglia/macrophages, and glucose dendrimers- to neurons) are somewhat secondary to the action on the specific receptors since the compositions target specific receptors. Important receptors include the serotonin receptors [5HT1A, 5HT2A (agonism, antagonism, reverse or inverse agonism)] and NMDA receptors. In many cases, binding to specific receptors and not binding to other receptors using dendrimers can enhance binding efficacy and reduce side effects of these drugs.

When combined with the cellular targeting capability of glucose dendrimers, such as to injured neurons (primary), microglia/macrophages (secondary), or hydroxyl dendrimers (microglia/macrophages), improvement in water solubility of >5-200-fold is shown with dendrimer conjugation, there are clear benefits to these conjugates: modifiable binding, increased selectivity of targeting, increased ease of formulation and delivery, and reduced side effects.

Using Tryptamine as an example, it was shown that binding affinities of drugs conjugated with these dendrimers have unexpected properties.

Both hydroxyl and Glucose dendrimers have OH surface groups. When tryptamine was conjugated to these dendrimers with the same linking chemistry, very different affinities were seen to serotonin and other receptors. The conjugates showed less affinity than free drug in cell-based binding assays (indicative of in vivo efficacies). The lower affinity may enable less tight binding and may enable us to modulate the undesirably strong effects of the drugs on this receptor. Second, the hydroxyl dendrimer conjugate was not active, but the glucose dendrimer conjugate was active. This was not expected and may be due to the differential internal structure of the glucose and hydroxyl dendrimers. The drug may fold into the hydrophobic core of the hydroxyl dendrimer but may open outwards in the hydrophilic interior of the glucose dendrimer.

### Example 5: Treatment of Rett Syndrome Animal Model with Ketamine and Ketamine-Dendrimer Conjugates

Rett syndrome (RTT) is an inherited neurodevelopmental disorder of females that occurs once in 10,000-15,000 births. Affected females develop normally for 6-18 months, but then lose voluntary movements, including speech and hand skills. Most RTT patients are heterozygous for mutations in the X-linked gene MECP2, encoding a protein that binds to methylated sites in genomic DNA and facilitates gene silencing. The symptoms, progression, and severity of Rett syndrome can vary dramatically from one person to another. A wide range of disability can potentially be associated with Rett syndrome. Symptoms generally appear in stages. RTT is typically characterized by a period of normal development after birth, followed by regression in speech and hand movements, gait abnormalities, erratic hand movements, and deceleration of head growth. Other diagnostic criteria for RTT include irregular breathing, gastrointestinal and musculoskeletal disorders, seizures, poor sleep, reduced response to physical pain, and behavioral issues.

Ketamine is a well established anesthetic drug that results in ‘dissociative anesthesia’ and exerts both central and peripheral effects including hypnosis, analgesia and sympathomimetic effects leading to hypertension and tachycardia. The main mechanism is felt to be due to its role as an antagonist at the N-methyl-d-aspartate (NMDA) receptor. This can lead to rapid action and response seen with treatment for treatment resistant depression, MDD and suicidal ideations, unlike SSRIs and SNRIs that only show delayed effects in controlling depression. Ketamine is also potent as a therapy in chronic pain, again due to its effects on NMDAR inhibition. Increased dopamine release and its role on However, ketamine also exerts neuroprotective effects by non-NMDAR mediated mechanisms such as increasing BDNF and mTOR. Ketamine also exerts effects on opioid receptors, can increase dopamine. These effects are beneficial for chronic pain, treatment resistant depression, MDD, and treatment resistant epilepsy/seizures.

However, ketamine has several short term and long term side effects. Ketamine can lead to respiratory depression at high doses, can lead to systemic side effects such as increased heart rate, hypertension, hyperthermia, loos of coordination, dizziness, nausea, vomiting, disturbing laternations in sensory perceptions and high incidence of auditory and visual hallucinations. More than half the patients who are treated with ketamine develop and emergence phenomenon when ketamine wears off that is characterized by euphoria, vivid dreams, hallucinations, illusions, distortions in body images and objects and delirium that can be extremely disturbing and may lead to self injury. Some of these symptoms correlate with symptoms of schizophrenia. Long term use of ketamine can lead to memory impairments and decline in executive functioning. Ketamine also leads to tolerance and is addictive leading to withdrawal and dependence. Due to these significant side effects, ketamine can only be administered in a controlled setting.

Ketamine has been shown to be effective in a mouse model of Rett syndrome at a large dose of 8 mg/kg delivered IP every day for 40 days (total dose of 320 mg/kg) and improved survival by 50% at 80 days post natal (Patrizi, et al. 2016)

### Materials and Methods

Dendrimer conjugates were prepared as described above.

As reported by Guy, et al. a mouse Mecp2-null mutation causes neurological symptoms that mimic Rett syndrome. Nat Genet. 2001 March; 27 (3): 322-6. doi: 10.1038/85899. PMID: 11242117 Both Mecp2-null mice and mice in which Mecp2 was deleted in brain show severe neurological symptoms at approximately six weeks of age.

Patrizi A, et al. Chronic Administration of the N-Methyl-D-Aspartate Receptor Antagonist Ketamine Improves Rett Syndrome Phenotype. Biol Psychiatry. 2016 May 1; 79 (9): 755-764. doi: 10.1016/j.biopsych.2015.08.018. Epub 2015 Aug. 24. PMID: 26410354; PMCID: PMC7410367, reported on a systematic, randomized preclinical trial of chronic administration of low-dose (8 mg/kg, intraperitoneal) ketamine, an NMDAR antagonist, starting either early in development or at the onset of RTT phenotype in Mecp2-null mice. Mice were treated from day 15 to day 55 or day 30 to day 55 at 8 mg ketamine/kg/day ip (total dose of 320 mg/kg for 40 days or 200 mg/kg for 25 days). Treatment from day 30 was not effective while treatment from day 15 showed some efficacy at 320 mg/kg total dose.

### Experimental Paradigm for MECP2 Knock Out (“KO”) Mice:

Treatment from 28 days of age (4 weeks of age, symptomatic). Untreated animals die by around 55-60 days.

Mice were treated with ketamine or ketamine conjugated to dendrimer. Controls were wild type treatment with saline, knock out treated with saline.

Animals were administered treatment twice weekly ip in a 2.5 mg/kg/dose. Total dose at time of assessment (60 days of age) is about 22.5 mg/kg.

Dendrimer ketamine was tested in the Rett syndrome mouse model IP at a dose of 2.5 mg/kg (ketamine) biweekly for 8 weeks (total dose of ketamine 40 mg/kg).

4 weeks old Mecp2 KOs (knock outs) were randomly divided and treated biweekly for 8 weeks with saline or 2.5 mg/Kg ip of Ketamine or D-Ketamine (WT-saline, KO-saline, KO-Ketamine, KO D-Ketamine groups). The weekly neurobehavior was evaluated by recording their composite neurobehavior score (NBS) based on a scale that includes assessments of mobility, gait, paw clasping, tremors, and respiration on a scale of 0-3 each; the higher the score, the worse the phenotype. D-ketamine treated group showed slowed the progression of disease phenotype with better neurobehavior scores post treatment, whereas the untreated KOs did not.

| | |
|---|---|
| Score | Description |

Mobility

| | |
|---|---|
| 0 | normal |
| 1 | normal speed but not climbing (slower) |
| 2 | Slow/only moves when prodded (little movement) |
| 3 | No spontaneous movement even when prodded |

Gait

| | |
|---|---|
| 0 | normal speed but not climbing (slower) |
| 1 | waddle |
| 2 | waddle-fee pointed outwad/walking on toes |
| 3 | severe waddle, shuffling; unable to take clean |
| | steps |

Paw Clench

| | |
|---|---|
| 0 | no clench |
| 1 | little/infrequent (1 instance) |
| 2 | intermittent |
| 3 | constant |

Respiration

| | |
|---|---|
| 0 | normal |
| 1 | slow or fast hiccups |
| 2 | hiccups and irregular rate |
| 3 | apneas |

Open Field Test: The long-term behavioral changes in Saline (WT), Ketamine and D-Ketamine groups versus KO-saline at 8.5th week of treatment. The motor function was assessed by recording the mice in an open field arena (10.5″×19″×8″). Mice were recorded in the same room where they were housed to avoid the stress and variability in the test procedure. Each mouse was placed in a clean open field arena and was allowed to explore for 10 minutes, and activity was recorded. Animals were placed in same way in the open field 5 cm away facing the longer wall.

### Results

Animals were assessed for survival, neurobehavior score and activity (total distance traveled, speed, and time spent in corners).

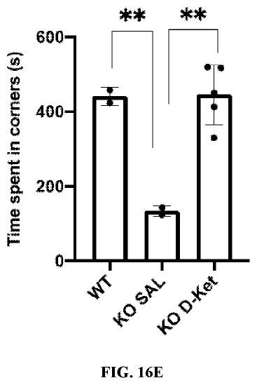

FIGS. 16A and 16B are graphs of wild type, knock out saline (controls) versus knockout mice treated with dendrimer-ketamine conjugate composite neurobehavior score of (FIG. 16A) and probability of survival over post natal day (FIG. 16B). FIG. 16C is a graph of the distance traveled (m); FIG. 16D is a graph of the speed at which the mice traveled; FIG. 16E is a graph of the time spent in corners.

Dendrimer conjugated with ketamine leads to increased efficacy with increased binding to NMDAR without the associated side effects. The dose used is significantly lower than free ketamine, which will reduce the side effects. This is tested in a mouse model of Rett syndrome as a proof of concept since Rett Syndrome is a disease that has increased glutamate production and increased NMDAR expression/activation.

Significant improvement in survival with 100% survival was seen up to 90 days of age with D-ketamine compared to untreated animals and animals treated with free ketamine. Previous published data by others in a similar model shows only 50% survival after treatment with 320 mg/kg of ketamine (8 times higher dose).

Significant improvement in motor function is seen with D-ketamine, with behavior similar to that of normal healthy controls.

Significant differences were seen in the total distance travelled by the WT versus KO mice in the open field for 10 minutes of duration. Significant improvement was also observed in motor function represented as the increment in the total distance travelled by the D-Ketamine treated KOs in open field test. Significant improvement were observed in maximum speed and time spent in corners by D-Ketamine in comparison to the KO Saline group.

Videos show dramatic improvement in phenotype bringing them close to healthy mice.

1. A composition comprising hydroxyl-terminated dendrimers, sugar-terminated dendrimers, and/or sugar-based dendrimers covalently conjugated to at least one or more psychedelic or hallucinogenic agents, optionally via a spacer, wherein the psychedelic or hallucinogenic agent is not a cannabinoid.

2. The composition of claim 1, wherein the dendrimers are generation 1, generation 2, generation 3, generation 4, generation 5, generation 6, generation 7, or generation 8 dendrimers.

3. The composition of claim 1, wherein the dendrimers are glucose dendrimers, preferably generation 1, generation 2, or generation 3 glucose dendrimers.

4. The composition of claim 1, wherein the dendrimers are hydroxyl-terminated poly (amidoamine) (PAMAM) dendrimers, preferably having greater than 40% or 50% hydroxyl surface groups.

5. The composition of claim 1, wherein the dendrimers are generation 3, generation 4, generation 5, generation 6, or generation 7, dendrimers.

6. The composition of claim 1, comprising sugar-terminated dendrimers comprising one or more monosaccharides on their surface selected from the groups consisting of glucose, galactose, glucosamine, galactose, mannose, and fructose, preferably greater than ten surface monosaccharide moieties.

7. The composition of claim 1, wherein the dendrimers are prepared from monosaccharide and optionally ethylene glycol building blocks, preferably made of galactose or glucose and optionally ethylene glycol and having greater than 10 surface sugar moieties.

8. The composition of claim 1, wherein the monosaccharide building blocks are one of more selected from the groups consisting of glucose, galactose, glucosamine, galactose, mannose, and fructose, wherein the dendrimer is preferably a glucose dendrimer.

9. The composition of claim 1, wherein the dendrimers are conjugated to one or more psychedelic agents via a spacer having a cleavable or non-cleavable linkage.

10. The composition of claim 9, wherein the cleavable bond is selected from the group consisting of ester, disulfide, phosphodiester, triglycyl peptide, and hydrazine linkages.

11. The composition of claim 9, wherein the non-cleavable bond is selected from the group consisting of amide, ether, and amino alkyl linkages.

12. The composition of claim 1, wherein the spacer linking the dendrimer and the psychedelic agent comprises a hydrocarbon such as an alkylene, a diethylene glycol moiety, and/or oligoethylene glycol chain.

13. The composition of claim 1, wherein the spacer comprises a triazole moiety.

14. The composition of claim 1, wherein the psychedelic agent is selected from the group consisting of classic serotonergic hallucinogens such as where the drug is an entactogen, dissociative anesthetic, or atypical hallucinogen.

15. The composition of claim 1, wherein the at least one or more psychedelic or hallucinogenic agents comprise psilocin, ketamine (R-ketamine, S-ketamine, (R/S)-ketamine), norketamine, ketamine metabolites, N,N dimethyl tryptamine (DMT), 4-acetoxy-N,N-dimethyl tryptamine, 5-methoxy DMT, 5-chloro DMT, lysergide (LSD), 3,4-methylenedioxymethamphetamine (MDMA), 3,4-methylenedioxyamphetamine (MDA),psilocybin, ibogaine, mescaline, mianserin, 2,5-dimethoxy-4-iodoamphetamine (DOI), ayahuasca, 1-(1-phencyclohexyl) piperidine (PCP), norbaeocystin, or analogues of derivatives of these agents.

16. The composition of claim 14, wherein the classic serotonergic hallucinogens are selected from the group consisting of psilocybin, psilocin, lysergic acid diethylamide (LSD), mescaline, mianserin and 2,5-dimethoxy-4-iodoamphetamine (DOI).

17. The composition of claim 14, wherein the entactogens are selected from the group consisting of 3,4-methylenedioxymethamphetamine (MDMA) and 3,4-methylenedioxyamphetamine (MDA).

18. The composition of claim 14, wherein the dissociative anesthetics are selected from the group consisting of ketamine (R-ketamine, S-ketamine, (R/S)-ketamine), norketamine, ketamine metabolites, 1-(1-phencyclohexyl) piperidine (PCP), or derivatives thereof.

19. The composition of claim 1, wherein the composition provides sustained release to yield an effective amount over a period of 24 hours.

20. The composition of claim 1, wherein the one or more psychedelic agents conjugated to the dendrimer are at a concentration of about 0.01% to about 30%, preferably about 1% to about 20%, more preferably about 5% to about 20% by weight.

21. The composition of claim 1, wherein the composition further comprises one or more diagnostic agents, preferably wherein the diagnostic agents are selected from the group consisting of fluorescent dyes, near infra-red dyes, SPECT imaging agents, PET imaging agents, and radioisotopes.

22. The composition of claim 1 providing sustained delivery of agents or sustained receptor activity at a certain level in order to achieve clinical benefit without hallucination or at a relatively constant dose to maintain a mental state, preferably at a therapeutically effective level for 24 hours.

23. A pharmaceutical formulation comprising the dendrimer of claim 1, and a pharmaceutically acceptable carrier or excipient.

24. The pharmaceutical formulation of claim 23, wherein the formulation is formulated for systemic administration.

25. The pharmaceutical formulation of claim 23, wherein the formulation is formulated for enteral or parenteral administration.

26. The pharmaceutical formulation of claim 23, wherein the formulation is formulated for oral, mucosal (intranasal, buccal, rectal, vaginal, sublingual, pulmonary), intramuscular, intravenous, subcutaneous, transdermal, or intrathecal administration.

27. The pharmaceutical composition of claim 23, in a form selected from the group consisting of hydrogels, nanoparticle or microparticles, suspensions, powders, tablets, capsules, creams, and solutions.

28. A method of treating one or more psychological, cognitive, behavioral, and/or mood disorders in a subject in need thereof comprising administering to the subject an effective amount of the composition of claim 1 or a pharmaceutical composition thereof, to treat, alleviate, and/or prevent one or more symptoms associated with the one or more of psychological, cognitive, behavioral, and/or mood disorders.

29. The method of claim 28, wherein the one or more of psychological, cognitive, behavioral, and/or mood disorders are selected from apathy, low motivation, attention disorders, disorders of executive function and/or cognitive engagement, obsessive compulsive disorder, neurocognitive disorders.

30. The method of claim 28, wherein the pharmaceutical composition is administered in an amount effective to provide improved motivation, attention, accuracy, speed of response, perseveration, and/or cognitive engagement, in the absence of an adverse side effect.

31. The method of claim 28, wherein the pharmaceutical composition is administered in an amount effective to provide binding of one or more psychedelic agents covalently conjugated to dendrimers to one or more receptors on the surface of or inside target cells.

32. The method of claim 31, wherein the target cells are neuronal cells, glial cells, macrophages of the peripheral and/or central nervous system.

33. The method of claim 31, wherein the receptors are selected from the group consisting of 5-HT receptor subtypes, adrenergic, dopaminergic, and histaminergic receptors.

34. The method of claim 33, wherein the receptors are selected from the group consisting of 5-HT₂, 5-HT₆, 5-HT₇, adrenergicα2, D1, and D2 receptors.

35. The method of claim 31, wherein the receptor is 5-HT_{2A}.

36. The method of claim 28 comprising treating a psychiatric disorder such as obsessive compulsive disorder, eating disorders, attention deficit and hyperactivity disorder, and schizophrenia.

37. The method of claim 28 comprising treating patients with depression such as treatment resistant depression, anxiety and post-traumatic stress disorder (PTSD).

38. The method of claim 28 comprising treating patients with drug and/or alcohol dependence.

39. The method of claim 28 comprising treating patients with Alzheimer's disease, dementia, multiple sclerosis or other neurodegenerative disorder.

40. The method of claim 28 comprising treating patients with chronic inflammation and autoimmune disorders such as rheumatoid arthritis, atherosclerosis, Parkinson's disease, Alzheimer's disease and multiple sclerosis.

41. The method of claim 28 comprising treating pain, including cluster headaches, migraines, chronic pain, post-operative pain, and cancer-related pain.

42. The method of claim 28 wherein the composition provides efficacy for a period of at least 24 hours after administration.

43. The method of claim 28, wherein the composition comprises glucose dendrimer-ketamine conjugate.
